(19) United States
(12) Patent Application Publication
(10) Pub. No.: US 2026/0263550 A1
(43) Pub. Date: Sep. 10, 2026

(54) METHODS AND COMPOSITIONS FOR TREATING, PREVENTING, INHIBITING, AMELIORATING OR DELAYING THE ONSET OF OPHTHALMIC CONDITIONS

(71) Applicant: Stealth BioTherapeutics Inc., Needham, MA (US)

(72) Inventors: Dennis Keefe, Needham, MA (US); Martin Redmon, Needham, MA (US); Brian Hotchkiss, Needham, MA (US); Anthony Abbruscato, Needham, MA (US)

(73) Assignee: Stealth BioTherapeutics Inc., Needham, MA (US)

(21) Appl. No.: 19/657,820

(22) Filed: Apr. 24, 2026

Related U.S. Application Data
(62) Division of application No. 18/460,346, filed on Sep. 1, 2023.
Application No. 18/460,346 is a continuation of application No. PCT/US2022/047172, filed on Oct. 19, 2022.
(60) Provisional application No. 63/257,738, filed on Oct. 20, 2021.
(60) Provisional application No. 63/331,412, filed on Apr. 15, 2022.

(30) Foreign Application Priority Data
Oct. 6, 2022 (WO) PCT/US2022/045908

(51) Int. Cl. A61K 38/05; A61P 27/02

(52) U.S. Cl. CPC A61K 38/05; A61P 27/02

(57) ABSTRACT

The disclosure generally relates to compounds (i.e. peptidomimetics), compositions (e.g. formulations or medicaments), methods and related uses for treating, preventing, inhibiting, amelioration or delaying the onset of ophthalmic diseases, disorders or conditions in a mammalian subject, such as a human. In some embodiments, the ophthalmic disease, disorder or condition may be associated with deterioration of the integrity of the ellipsoid zone of one or more eyes of the mammalian subject. The methods and uses comprise administering an effective amount of peptidomimetic (alone, as formulated and/or in combination with at least one additional therapeutic agent) to mammalian subjects in need thereof.

### CROSS-REFERENCE TO RELATED APPLICATIONS

This application is a divisional of U.S. patent application Ser. No. 18/460,346, filed on Sep. 1, 2023, which is a continuation of International Patent Application No. PCT/US2022/047172, filed on Oct. 19, 2022, which claims the benefit of U.S. Provisional Application No. 63/257,738 filed on Oct. 20, 2021, U.S. Provisional Application No. 63/331,412 filed on Apr. 15, 2022, and International Patent Application No. PCT/US2022/045908, filed on Oct. 6, 2022, the entire contents of each of which are incorporated herein by reference for all purposes.

### TECHNICAL FIELD

The present technology relates generally to compounds (i.e. peptidomimetics), compositions (e.g. medicaments) and methods for treating, preventing, inhibiting, amelioration or delaying the onset of ophthalmic diseases, disorders or conditions in a mammalian subject. In some embodiments, the ophthalmic disease, disorder or condition is associated with deterioration of the integrity of the ellipsoid zone of one or more eyes of the mammalian subject. For example, the present technology may relate to administering one or more mitochondrial-targeting peptidomimetics (alone, as formulated and/or in combination with other active pharmaceutical ingredients) in effective amounts to treat, prevent, inhibit, ameliorate or delay the onset of ophthalmic diseases, disorders or conditions (e.g., macular degeneration (including (wet or dry) age-related macular degeneration), dry eye, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (often referred to as CPEO or just PEO, e.g., Kearns-Sayre syndrome), and/or Leber congenital amaurosis (LCA)), in mammalian subjects.

### INTRODUCTION

The following introduction is provided to assist the understanding of the reader. None of the information provided, or references cited, is admitted as being prior art to the present technology.

Diseases, disorders and degenerative conditions of the optic nerve and retina are the leading causes of blindness in the world. Many ophthalmic diseases disorders or conditions result from, or are associated with, mitochondrial dysfunction.

A significant degenerative condition of the retina is age-related macular degeneration (AMD). AMD is the most common cause of blindness in people over the age of 50 in the United States and its prevalence increases with age. AMD is classified as either wet (neovascular) or dry (non-neovascular). The dry form of the disease is more common. Macular degeneration occurs when the central retina has become distorted and thinned. This change is usually associated with age but also characterized by intra-ocular inflammation and angiogenesis (wet AMD only) and/or intra-ocular infection. The subsequent generation of free radicals, resulting in oxidative tissue damage, local inflammation and production of growth factors (such as VEGF and FGF) and inflammatory mediators, can lead to inappropriate neovascularization in common with the wet form of AMD. Mitochondrial dysfunction is believed to play a role in age-related disorders such as AMD. (Liu et al., Appl. Sci. (2021) 11: 7385). Pieramici & Ehlers have reported that: “RPE mitochondria in AMD eyes undergo more pronounced degenerative changes, with lower mitochondrial density, organelle area and cristae number.” (Pieramici & Ehlers, Presentation at 54^{th} Annual Retina Society Meeting, Sep. 30, 2021, slide 3).

Retinopathy is a leading cause of blindness in type I diabetes and is also common in type II diabetes. The degree of retinopathy depends on the duration of diabetes, and generally begins to occur ten or more years after onset of diabetes. Diabetic retinopathy may be classified as non-proliferative, where the retinopathy is characterized by increased capillary permeability, edema and exudates, or proliferative, where the retinopathy is characterized by neovascularization extending from the retina to the vitreous, scarring, deposit of fibrous tissue and the potential for retinal detachment. Diabetic retinopathy is believed to be caused by the development of glycosylated proteins due to high blood glucose and leads to damage in small blood vessels in the eye. Diabetic retinopathy (often if left untreated) can progress to diabetic macular edema. Diabetic macular edema involves damage to the blood vessels in the retina that progress to a point where they leak fluid into the macula thereby causing the macula to swell and this results in blurred vision. Mitochondrial dysfunction has been linked to the pathogenesis of diabetic retinopathy. (Wu et al. Hindawi Oxidative Medicine and Cellular Longevity, Volume 2018, Article 3420187)

Glaucoma is made up of a collection of eye diseases that cause vision loss by damage to the optic nerve and retinal ganglion cells (RGCs). An intraocular pressure (IOP) of over 21 mmHg without optic nerve damage is known as ocular hypertension. Elevated IOP due to inadequate ocular drainage is the primary cause of glaucoma. Lowering IOP reduces the risk of progressive RGC loss in glaucoma; however, no currently available treatments directly prevent RGC damage. Glaucoma often develops as the eye ages, or it can occur as the result of an eye injury, inflammation, tumor or in advanced cases of cataract or diabetes. It can also be caused by the increase in IOP caused by treatment with steroids. Drug therapies that are proven to be effective in glaucoma reduce IOP either by decreasing vitreous humor production or by facilitating ocular draining. Such agents are often vasodilators and as such act on the sympathetic nervous system and include adrenergic antagonists. It has been stated that: “ . . . mitochondrial dysfunction plays an important role in the pathogenesis of neurodegenerative diseases . . . ” and “ . . . mitochondrial damage may provide potential strategies for the treatment of glaucoma . . . ” (Liu et al., Appl. Sci. (2021) 11: 7385).

Autosomal dominant optic atrophy (DOA) is a genetic X-linked neuro-ophthalmic condition characterized by bilateral degeneration of optic nerves. It affects approximately 1 in 10,000 (Denmark) to 1 in 30,000 (worldwide) persons. The nerve damage causes visual loss. It generally begins to manifest itself during the first decade of life and progresses thereafter. The disease itself affects primarily the retinal ganglion nerves. Mutations in the genes known as OPA1 and OPA3, which encode inner mitochondrial membrane proteins (resulting in mitochondrial dysfunction), are generally associated with DOA.

Leber Hereditary Optic Neuropathy (LHON) is a genetically-based inherited disease that generally starts to manifest itself between the ages of 15 and 35. In LHON, mitochondrial mutations affect complex I subunit genes in the respiratory chain leading to selective degeneration of retinal ganglion cells (RGCs) and optic atrophy generally within a year of disease onset. LHON is caused by mutations in the MT-NDI1, MT-ND4, MT-ND4L and MT-ND6 genes; all of which are associated with mitochondrial genome coding. LHOH affects approximately 1 in 50,000 people worldwide. It generally starts in one eye and progresses quickly to the other eye. Subjects with LHON may eventually become legally or totally blind, often before they turn 50. LHON affects vision needed for tasks such as reading, driving and recognizing others.

Retinitis pigmentosa (RP) is a group of hereditary retinal degenerative disorders characterized by progressive vision loss. RP is a leading cause of inherited blindness in the developed world. Clinically, RP is manifested by night vision difficulties due to the death of rod photoreceptors followed by the progressive loss of peripheral vision eventually leading to central vision impairment from the secondary loss of cone photoreceptors. RP is caused by mutations of at least 87 genes. The pathogenesis of RP is not well understood. However, mitochondrial dysfunction and oxidative damage are believed to play a key role in the pathogenesis of photoreceptor cell death in RP. (Gopalakrishnan et al., Scientific Reports (2020) 10: 20382)

Pigmentary retinopathy (PR) is a frequent feature of retinitis pigmentosa. Pigmentary retinopathy is a non-specific finding that may be found in several mitochondrial diseases, such as Neurogenic weakness, Ataxia, and Retinitis Pigmentosa (NARP). PR is an inherited degenerative disorder of the retina, characterized by progressive photoreceptor damage. The damage leads to atrophy and cell death of the photoreceptors. Patients with PR can follow an autosomal-dominate, autosomal recessive or X-linked recessive pattern. The prevalence is about one in about three to four thousand individuals. Symptoms of the disease include nyctalopia (night blindness), peripheral visual field constriction, and sometimes loss of the central visual acuity or visual field.

Uveitis is array of intraocular inflammatory diseases of the eye that often results in irreversible visual loss. Uveitis is responsible for an estimated 30,000 new cases of legal blindness annually in the USA. It is believed that this disease is at least in part due to retinal tissue damage caused excessive mitochondrial oxidative stress that triggers a damaging immune response.

Chronic progressive external ophthalmoplegia (CPEO) is a condition characterized mainly by a loss of the muscle functions including in eye and eyelid movement. The condition typically appears in adults between ages 18 and 40 and slowly worsens over time. CPEO can be caused by genetic changes in any of several genes, which may be located in mitochondrial DNA or nuclear DNA. CPEO can occur as part of other underlying conditions, such as ataxia neuropathy spectrum and Kearns-Sayre syndrome. These conditions may not only involve CPEO, but various additional features that are not shared by most individuals with CPEO.

Kearns-Sayre syndrome is a condition that affects many parts of the body, especially the eyes. The features of Kearns-Sayre syndrome usually appear before age 20, and the condition is diagnosed by a few characteristic signs and symptoms. People with Kearns-Sayre syndrome have progressive external ophthalmoplegia. Affected individuals also have an eye condition called pigmentary retinopathy, which results from breakdown (degeneration) of the retina that gives it a speckled and streaked appearance.

Leber congenital amaurosis (LCA) is a rare genetic eye disorder that affects infants. The infants are often blind at birth. LCA can be associated with mitochondrial dysfunction. (Castro-Gago et al., J. Child Neurol. (1996) 11(2):108-11) Children born with LCA have light-gathering cells (rods and cones) of the retina that do not function properly. LCA has been estimated to be 1-2/100,000 births. This disorder affects males and females in equal numbers.

Drusen are small yellow or white spots between the retinal pigment epithelium and Bruch's membrane in the retina that can be detected by an ophthalmologist during a dilated eye exam or with retinal photography. Drusen can also be imaged and monitored by optical coherence tomography (OCT). Drusen are made up of lipids and proteins. Drusen are a defining feature of macular degeneration. Drusen can be hard or soft. Larger numbers of drusen, as well as drusen of larger size, indicate higher risk for some vision loss in the future. “Hard” drusen are small and indicate lower risk of future vision loss than “soft” drusen. “Soft” drusen are larger, cluster together, and have edges that are not as clearly defined. Soft drusen are more likely to lead to vision loss.

Geometric Atrophy (GA) is generally considered part of the later stage of age-related macular degeneration (AMD) and refers to progression of the disease to a point where in regions of the retina, cells begin to waste away and die (i.e. atrophy).

Best corrected visual acuity (BCVA) is a measure of the best possible vision an eye can achieve with the use of glasses or corrective lenses. It is typically measured using Snellen lines on an eye chart. Repeated testing of the BCVA over time can be used to determine if a subject's vision is stable, improving or deteriorating.

Low luminance visual acuity (LLVA) involves standard visual acuity testing under low-light conditions. This is often achieved by adding a neutral density filter in front of the testing eye. It is a useful visual function marker in those with geographic atrophy (GA) and neovascular age-related macular degeneration. Repeated testing of the LLVA over time can be used to determine if a subject's vision, under low light conditions, is stable, improving or deteriorating.

Optical coherence tomography (OCT) is a non-invasive imaging method used to generate a picture of the back of the eye (i.e. the retina). OCT uses a low-powered laser to create pictures of the layers of the retina and optic nerve. The cross-sectional images are three-dimensional and color-coded. OCT can measure the thickness of the retina and optic nerve. OCT can be used to diagnose and manage Glaucoma, AMD, diabetes-related retinopathy, cystoid macular edema, macula pucker and macular hole.

Spectral domain optical coherence tomography (SDOCT) is an interferometric technique that provides depth-resolved tissue structure information encoded in the magnitude and delay of the back-scattered light by spectral analysis of the interference fringe pattern. SDOCT increases axial resolution 2- to 3-fold and scan speed 60- to 110-fold vs conventional (TD) OCT.

The ellipsoid zone can be mapped using SCOCT and the integrity of (or changes in) the ellipsoid zone can be determined from such mapping/scanning activity. (Itoh et al., Br J Ophthalmol. (2016) 100(3): 295-299). The technology is capable of evaluating the structures of the external limiting membrane (ELM), ellipsoid zone (EZ), interdigitation zone (IZ) and the retinal pigment epithelium (RPE). Id. Use of this technology is capable of accessing EZ integrity and EZ-RPE alterations. Id. The EZ and ELM, in particular, have been linked to visual outcomes and prognosis in numerous macular conditions, such as age-related macular degeneration (AMD) Id Itoh et al. suggest that the utility of SDOCT as an assessment tool for EZ integrity for clinical trials and disease prognostication/management may prove particularly useful.

Swept source OCT (SS-OCT) and OCT angiography (OCTA) are relatively new techniques that are capable of better resolution of the retinal pigment epithelium (RPE), Bruch's membrane (BM) and choriocapillaris (CC) structures. (Zhou et al. Biomedical Optics Express (2020) 11(4): 1834-1850) Using this technology it is possible to generate relative distance and thickness maps of the RPE-BM-CC complex. Id Use of these techniques may provide a better understanding of the CC in three dimensions, and further investigate potential functional relationships between RPE, BM and CC, and their involvement in age-related ocular diseases. Id.

The ellipsoid zone (EZ) of the eye is a mitochondrial rich tissue (Ball et al., Sci. Adv. 8, eabn2070 (2022)). The ellipsoid zone can be imaged using optical coherence tomography (Fujita et al., Scientific Reports (2019) 9:12433). The integrity of the EZ can be quantified. (Fugita et al.). There is a clear relationship between the integrity of the ellipsoid zone and visual function. (Fugita et al., Figure. 3). Ball et al. suggest that tightly packed mitochondria in the ellipsoid “focus” light for entry into the outer segment and that healthy mitochondria structure (including cristae structure) might be important for producing a Stiles-Crawford effect (SCE) and maintaining visual resolution in mammals. Pieramici & Ehlers describe mapping the ellipsoid zone to thereby observe the ellipsoid zone and possibly monitor changes in the integrity of the ellipsoid zone. (Pieramici & Ehlers, Presentation at 54^{th} Annual Retina Society Meeting, Sep. 30, 2021). Pieramici & Ehlers further described the use of Sub-RPE compartment maps as a means to find and monitor drusen formation and RPE atrophy in a subject. In the study being described (which described results from a P2 clinical trial involving treatments with elamipretide), Pieramici & Ehlers concluded, inter alia, that: (i) “Average BCVA and LLVA in NCGA and HRD patients improved significantly at 24 weeks [of treatment with elamipretide]” and (ii) “Baseline higher order OCT parameters, such as EZ integrity, correlated with improved LLVA in Elamipretide-treated eyes” (Pieramici & Ehlers at slide 15).

In brief, there are many ophthalmic diseases for which there remains a need for treatments/therapies or improved treatments/therapies. For example, there remains a need for treatments/therapies, or improved treatments/therapies, to address ophthalmic diseases, disorders or conditions such as macular degeneration (including (wet or dry) age-related macular degeneration), dry eye, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (e.g., Kearns-Sayre syndrome), and/or Leber congenital amaurosis (LCA). This forgoing discussion addresses these needs.

### SUMMARY

The present technology relates generally to the treatment, prevention, inhibition, amelioration or delaying the onset of ophthalmic diseases, disorders or conditions in mammals through administration of a therapeutically effective amount of at least one peptidomimetic to a subject in need thereof. Such peptidomimetic can be a mitochondrial-targeting peptidomimetic. For example, such peptidomimetic can be a compound of Formula I (defined below), or a pharmaceutically acceptable salt, stereoisomer, tautomer, hydrate, and/or solvate thereof. In some embodiments, the peptidomimetic is a compound of Formula II (defined below), such as a tris-HCl salt of Formula II (identified below as Formula IIa). In some embodiments, the peptidomimetic is a compound of Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof.

For example, in one aspect, the present disclosure provides a method of treating, preventing, inhibiting, amelioration or delaying the onset of an ophthalmic disease, disorder or condition in a mammalian subject in need thereof, the method comprising administering to the subject a therapeutically effective amount of at least one peptidomimetic, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

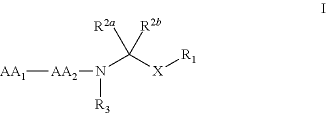

wherein,
AA₁ is selected from

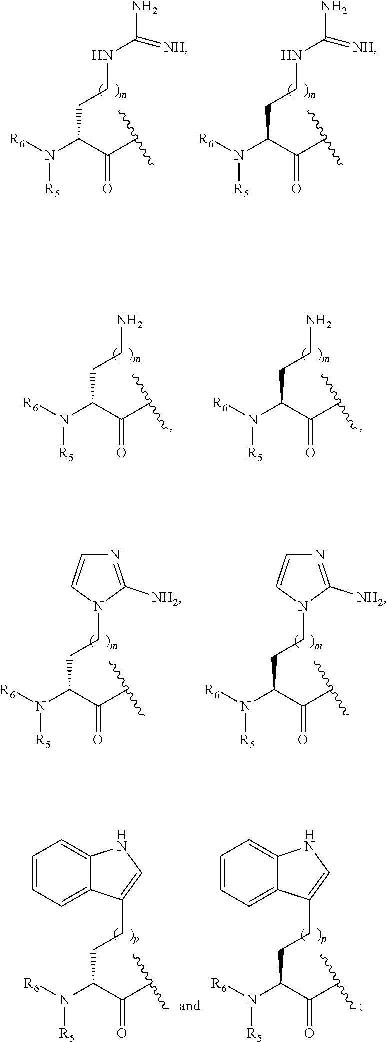

AA₂ is selected from

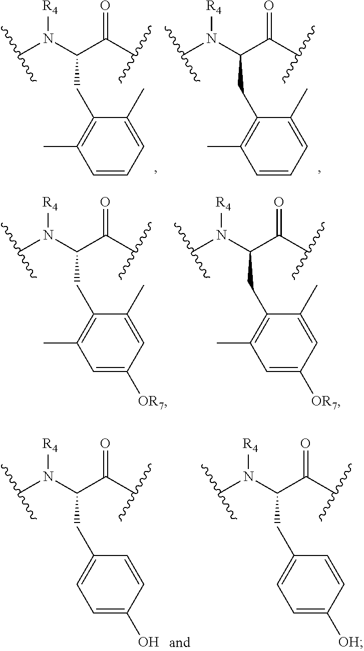

R₁ is selected from

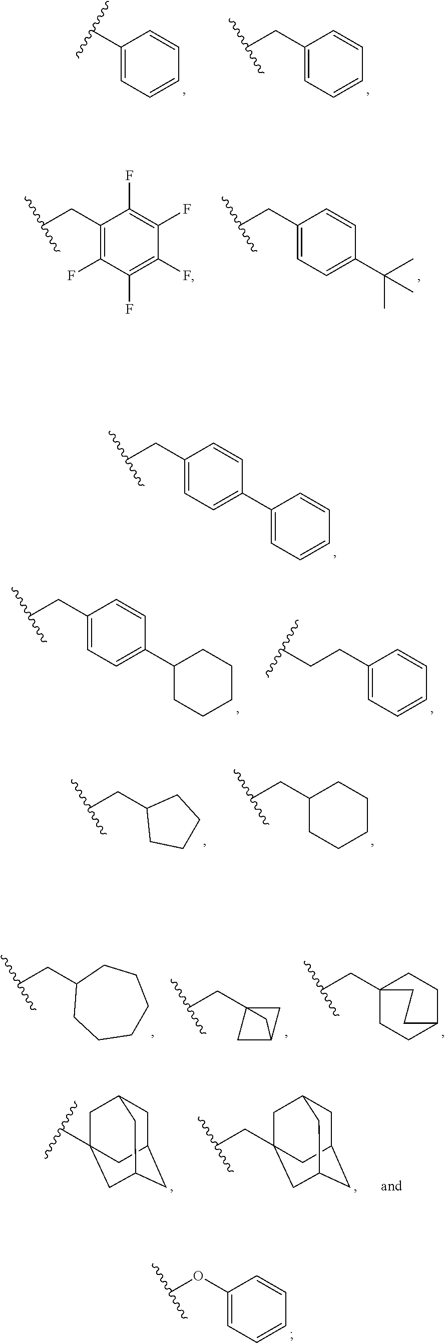

R^{2a} is selected from

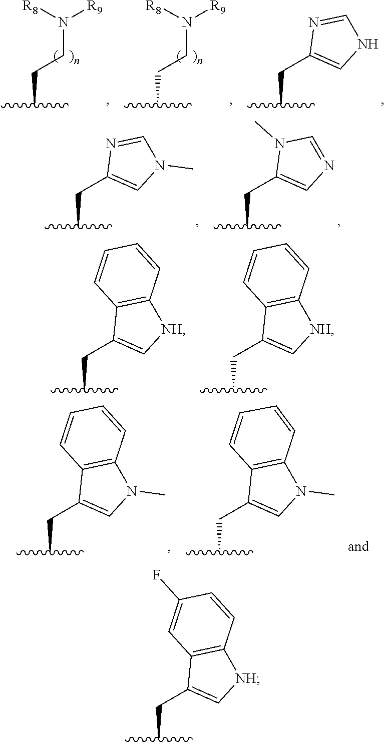

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

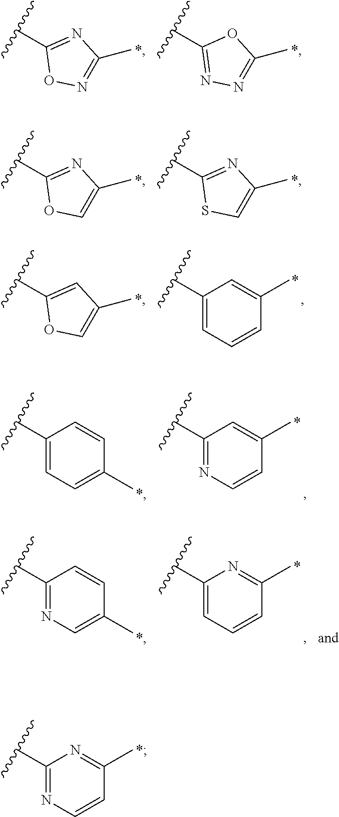

and
* denotes the point of attachment of X to R₁, and wherein one or more of the hydrogen atoms of the peptidomimetic is optionally substituted with a deuterium or fluorine atom.

In one aspect, the present disclosure provides for use of a composition in the preparation of a medicament for treating, preventing, inhibiting, ameliorating or delaying the onset of: (i) an ophthalmic disease, disorder or condition; or (ii) deterioration of ellipsoid zone integrity in one or more eyes in a mammalian subject in need thereof, wherein the composition comprises a therapeutically effective amount of at least one peptidomimetic, or a pharmaceutically acceptable salt, stereoisomer, tautomer, hydrate, and/or solvate thereof. For example, the peptidomimetic can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

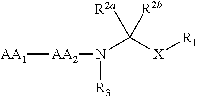

wherein,
AA₁ is selected from

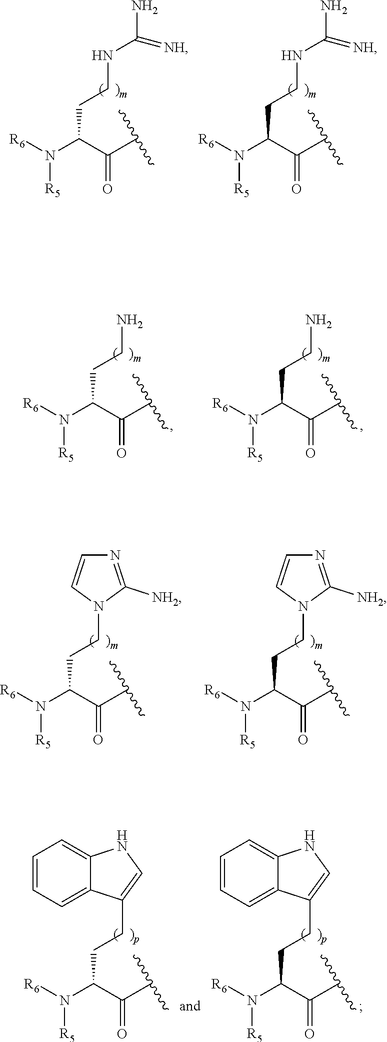

AA₂ is selected from

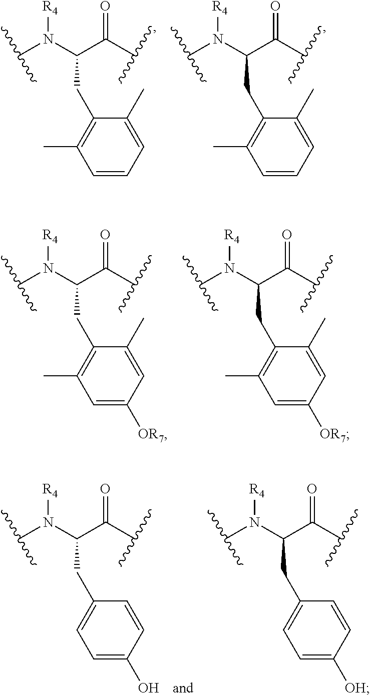

R₁ is selected from

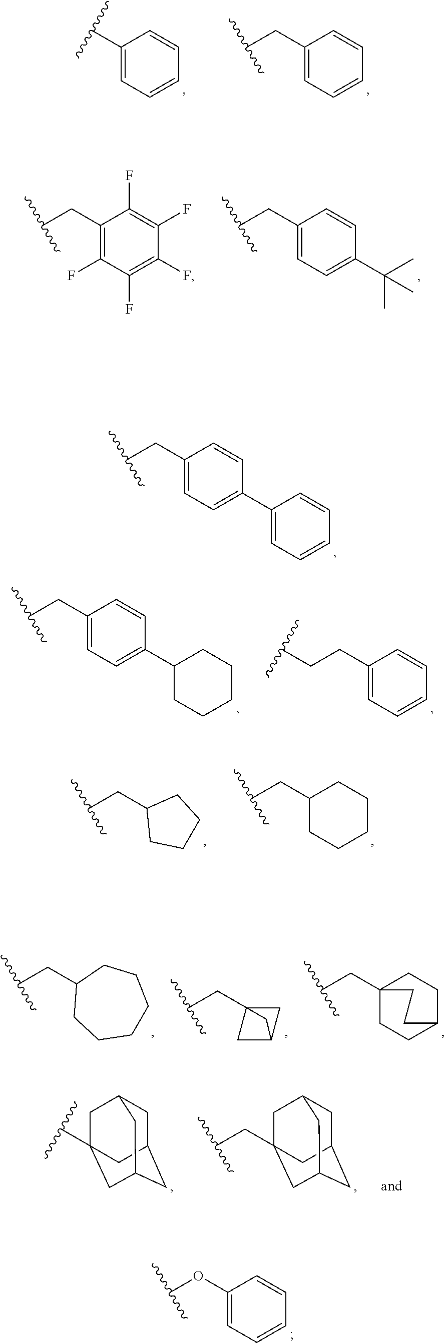

R^{2a} is selected from

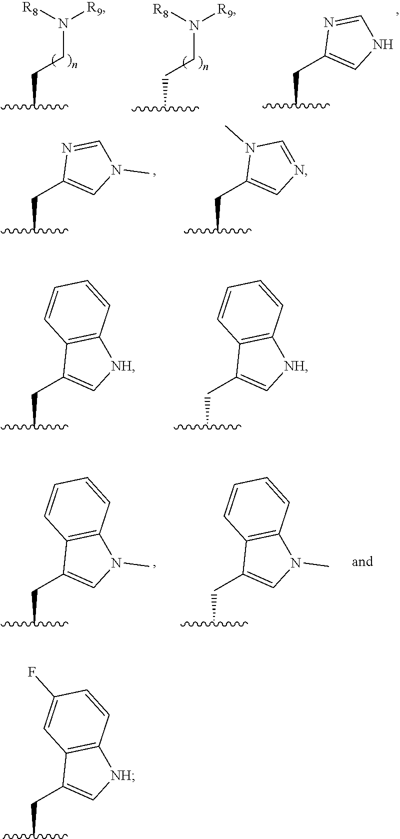

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

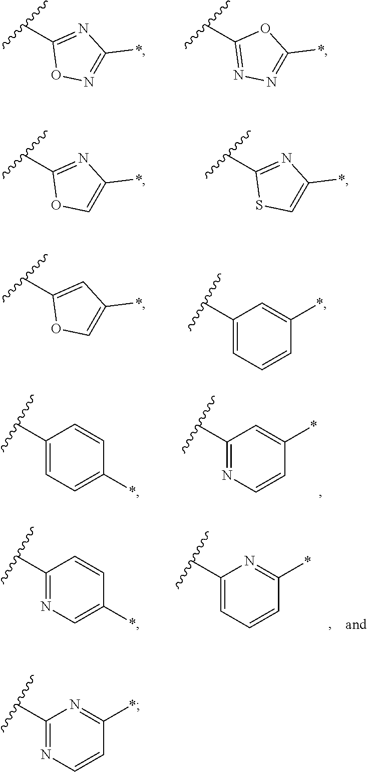

and
* denotes the point of attachment of X to R₁, and wherein one or more of the hydrogen atoms of the peptidomimetic is optionally substituted with a deuterium or fluorine atom.

In some embodiments, the composition is produced by dissolving or suspending the peptidomimetic in a diluent, adjuvant, excipient, or vehicle, such as water or a solvent mixture comprising water. In some embodiments, the composition or medicament further comprises a preservative. In some embodiments, the preservative is present in the composition or medicament in a concentration of less than 1% (wt./vol.). In some embodiments, the peptidomimetic(s) is/are present in the composition or medicament at a concentration of less than 1% (wt./vol.). In some embodiments, the peptidomimetic(s) is/are present in the composition or medicament in a concentration of between 0.5 and 1% (wt./vol.), inclusive. In some embodiments, the peptidomimetic(s) is/are present in the composition or medicament in a concentration of between 1 and 2% (wt./vol.), inclusive. In some embodiments, the peptidomimetic(s) is/are present in the composition or medicament in a concentration of between 2 and 3% (wt./vol.), inclusive. In some embodiments, the peptidomimetic(s) is/are present in the medicament in a concentration of between 3 and 5% (wt./vol.), inclusive. In some embodiments, the peptidomimetic(s) is/are present in the medicament in a concentration above 5% (wt./vol.). In some embodiments, the peptidomimetic(s) is/are present in the medicament in a concentration above 10% (wt./vol.).

In one aspect, the present disclosure provides a formulation or medicament for treating, preventing, inhibiting, ameliorating or delaying the onset of: (i) an ophthalmic disease, disorder condition; or (ii) deterioration of ellipsoid zone integrity in one or more eyes in a mammalian subject in need thereof, said formulation or medicament comprising a therapeutically effective amount of at least one peptidomimetic, or a pharmaceutically acceptable salt, stereoisomer, tautomer, hydrate, and/or solvate thereof. For example, the peptidomimetic used in the formulation can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. (Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

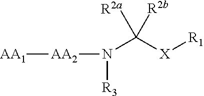

wherein,
AA₁ is selected from

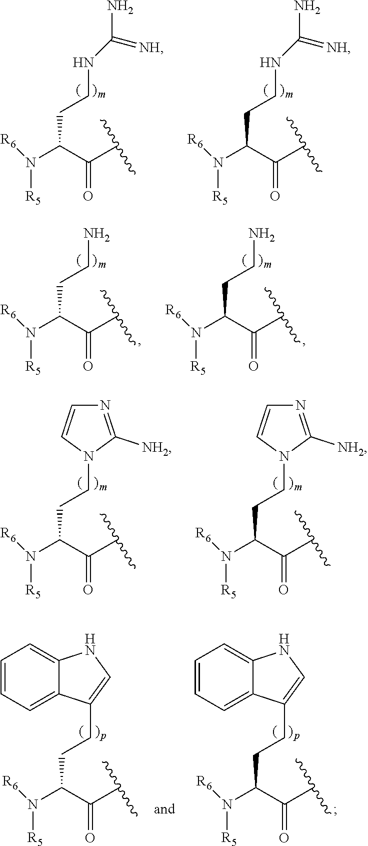

AA₂ is selected from

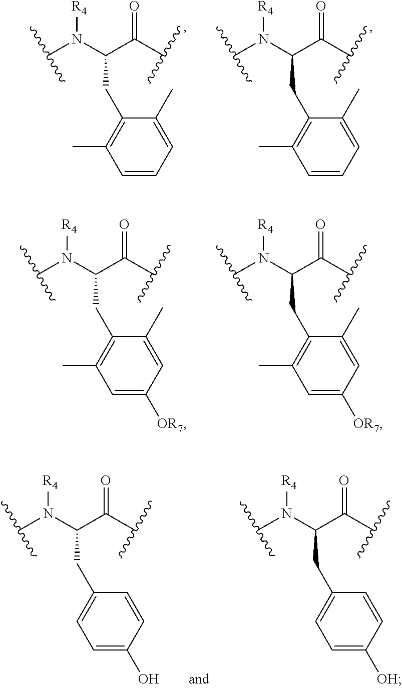

R₁ is selected from

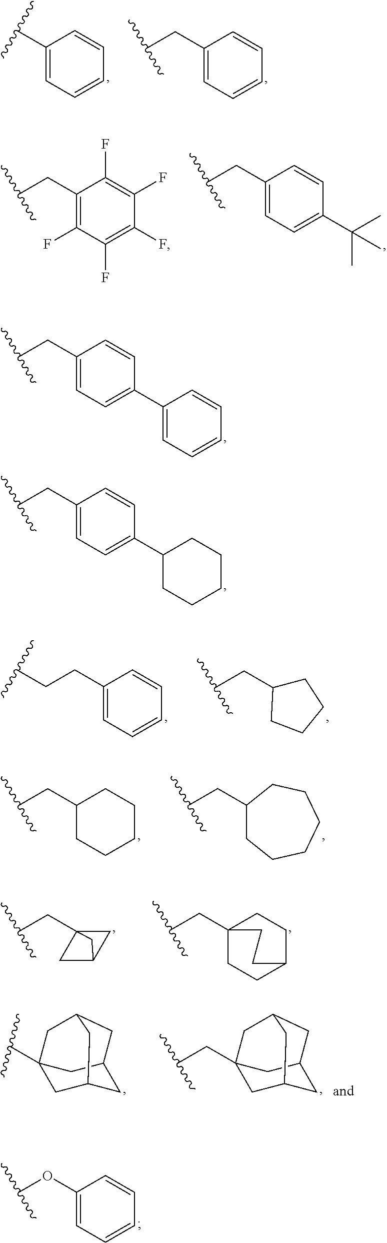

R^{2a} is selected from

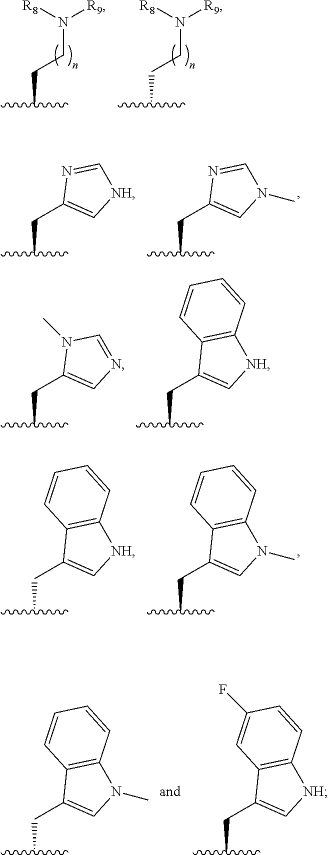

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

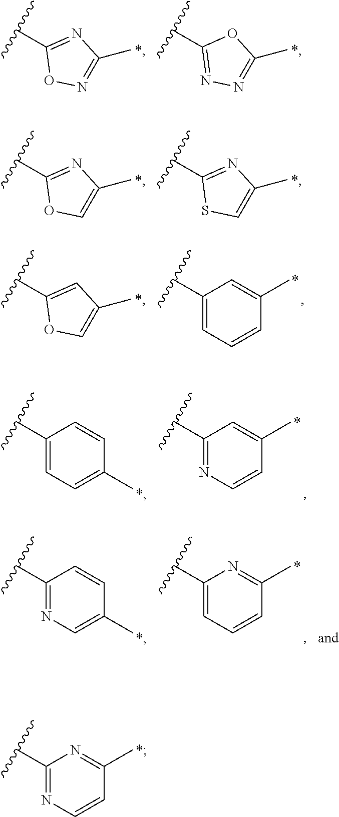

and
* denotes the point of attachment of X to R₁, and wherein one or more of the hydrogen atoms of the peptidomimetic is optionally substituted with a deuterium or fluorine atom.

In some embodiments, the formulation or medicament is produced by dissolving or suspending the peptidomimetic in a diluent, adjuvant, excipient, or vehicle, such as water or a solvent mixture comprising water. In some embodiments, the formulation or medicament further comprises a preservative. In some embodiments, the preservative is present in the formulation or medicament in a concentration of less than 1% (wt./vol.). In some embodiments, the peptidomimetic(s) is/are present in the formulation or medicament at a concentration of less than 1% (wt./vol.). In some embodiments, the peptidomimetic(s) is/are present in the formulation or medicament in a concentration of between 0.5 and 1% (wt./vol.), inclusive. In some embodiments, the peptidomimetic(s) is/are is present in the formulation or medicament in a concentration of between 1 and 2% (wt./vol.), inclusive. In some embodiments, the peptidomimetic(s) is/are present in the formulation or medicament in a concentration of between 2 and 3% (wt./vol.), inclusive. In some embodiments, the peptidomimetic(s) is/are present in the formulation or medicament in a concentration of between 3 and 5% (wt./vol.), inclusive. In some embodiments, the peptidomimetic(s) is/are present in the formulation or medicament in a concentration above 5% (wt./vol.). In some embodiments, the peptidomimetic(s) is/are present in the formulation or medicament in a concentration above 10% (wt./vol.).

In one aspect, the present disclosure provides a method for treating, preventing, inhibiting, ameliorating or delaying the onset of deterioration of ellipsoid zone integrity in one or more eyes of a mammalian subject in need thereof, comprising administering to the subject a therapeutically effective amount of at least one peptidomimetic. For example, the peptidomimetic can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (II), or a pharmaceutically acceptable salt (e.g. (IIa)), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

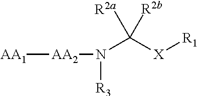

wherein,
AA₁ is selected from

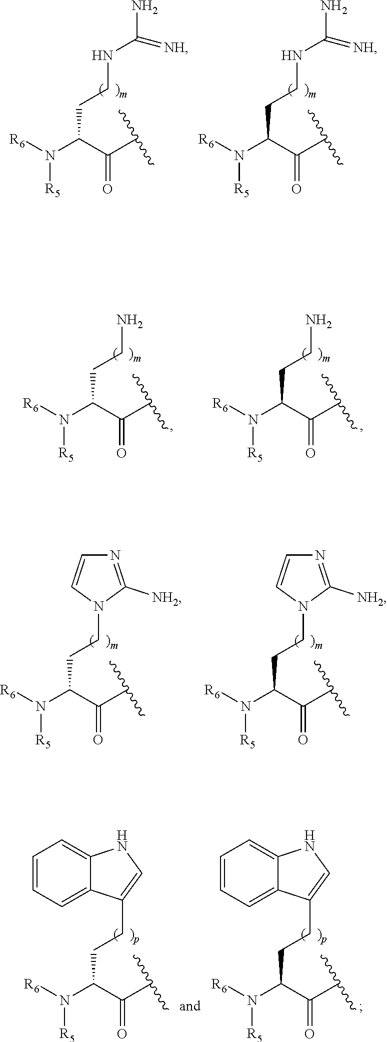

AA₂ is selected from

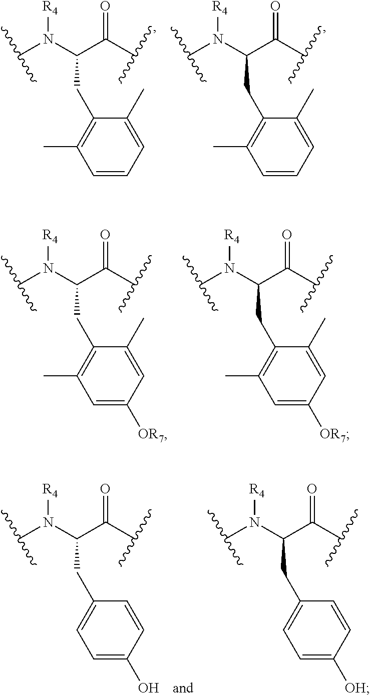

R₁ is selected from

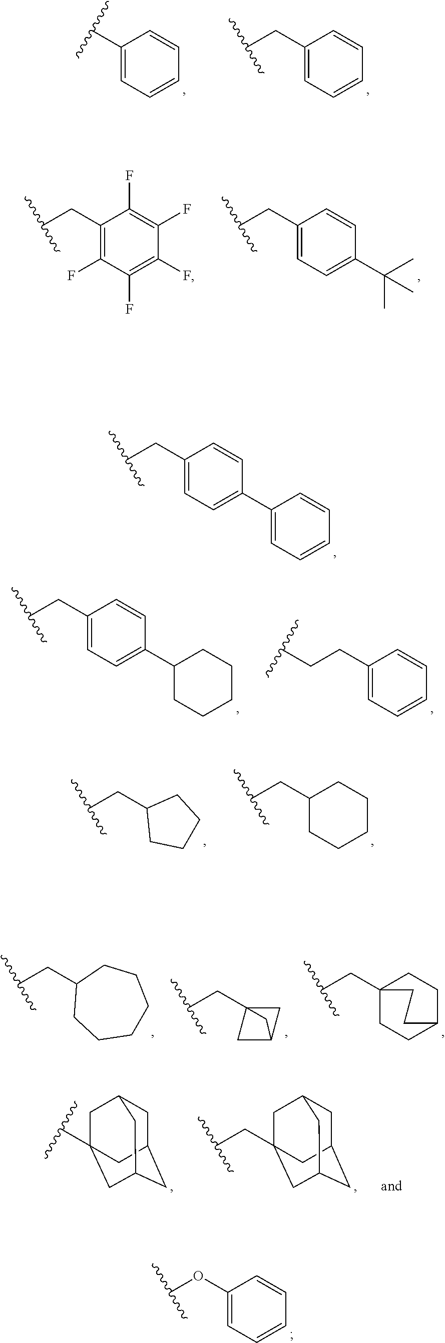

R^{2a} is selected from

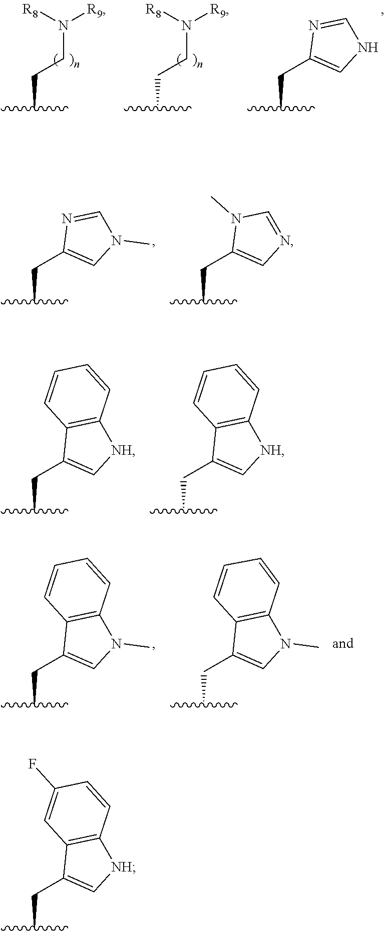

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

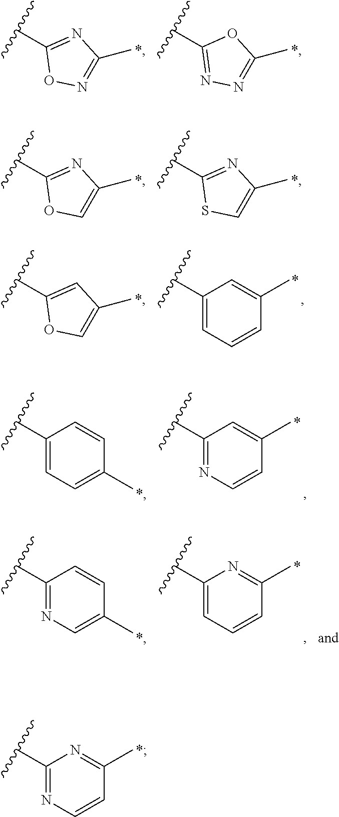

and
* denotes the point of attachment of X to R₁, and wherein one or more of the hydrogen atoms of the peptidomimetic is optionally substituted with a deuterium or fluorine atom.

In one aspect, the present disclosure provides a method for treating, preventing, inhibiting, ameliorating or delaying the onset of geometric atrophy a mammalian subject in need thereof where the subject has been diagnosed with age-related macular degeneration (AMD), comprising administering to the subject a therapeutically effective amount of at least one peptidomimetic. For example, the peptidomimetic can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (II), or a pharmaceutically acceptable salt (e.g. (IIa)), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

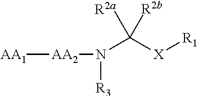

wherein,
AA₁ is selected from

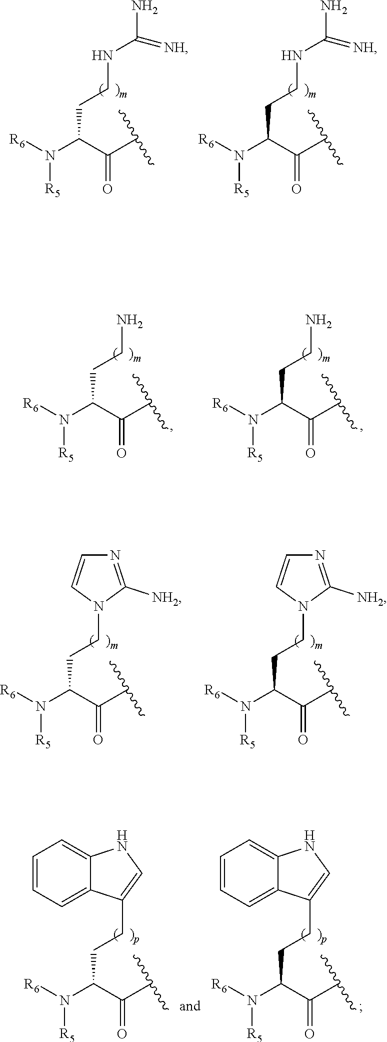

AA₂ is selected from

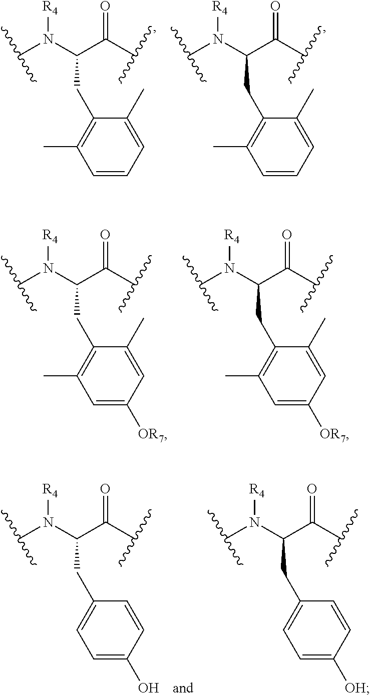

R₁ is selected from

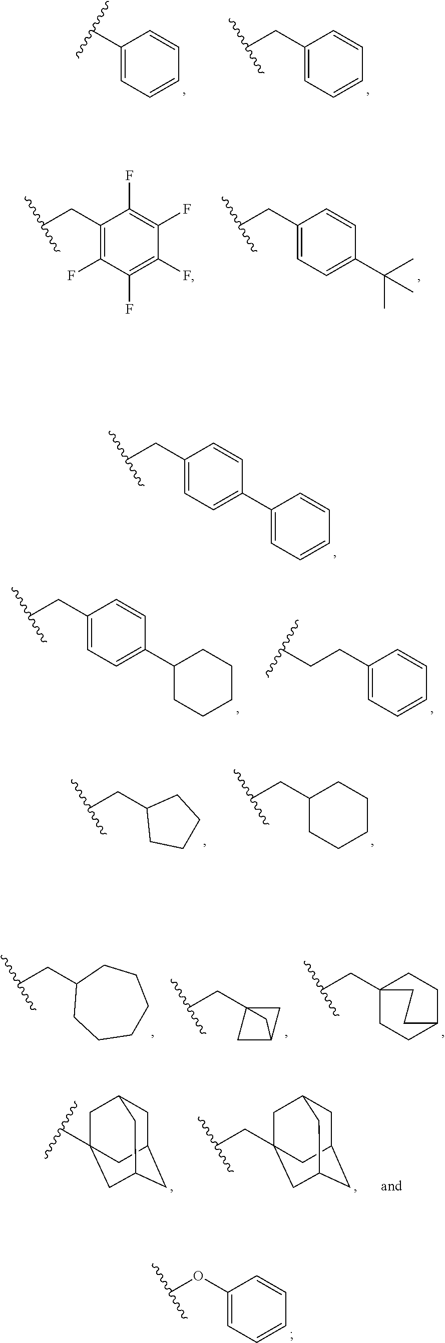

R^{2a} is selected from

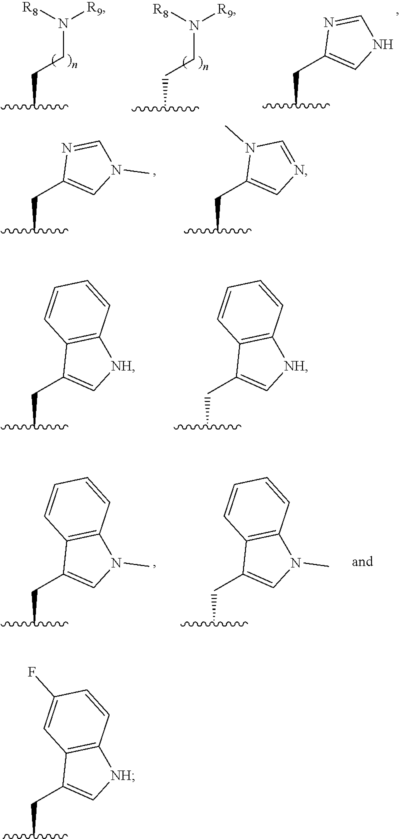

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

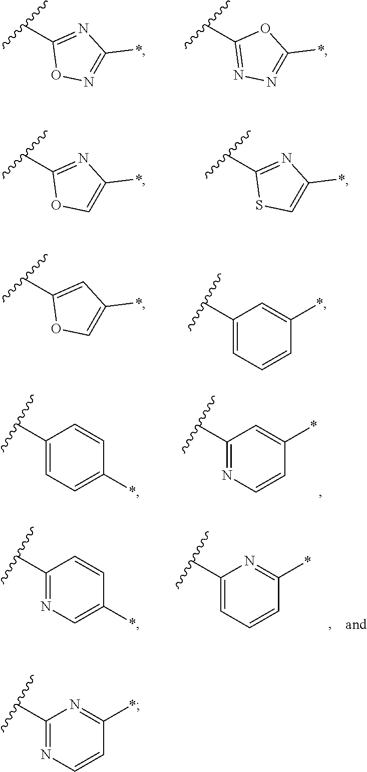

and
* denotes the point of attachment of X to R₁, and wherein one or more of the hydrogen atoms of the peptidomimetic is optionally substituted with a deuterium or fluorine atom.

In some embodiments of the foregoing methods, uses, compositions, formulations or medicaments, the peptidomimetic is a peptidomimetic of Formula I, wherein AA₁ is selected from

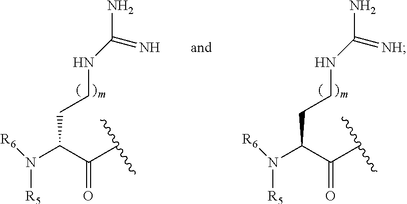

AA₂ is selected from

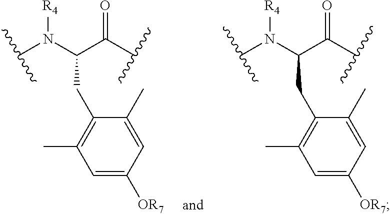

R₁ is selected from

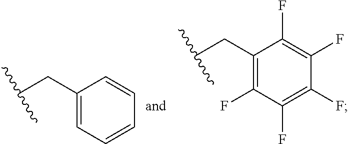 [SMILES: *Cc1ccccc1 ; *Cc1c(F)c(F)c(F)c(F)c1F]

R^{2a} is selected from

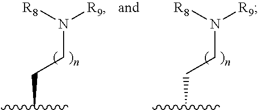

R^{2b} is H; R₃ and R₄ are independently selected from H and methyl; R₅ and R₆ are independently selected from H and methyl; R₇ is selected from H and methyl; R₈ and R₉ are independently selected from H and methyl; and
X is selected from

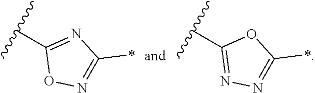

In some embodiments of the foregoing methods, uses, compositions, formulations or medicaments, the peptidomimetic is a peptidomimetic of Formula I, wherein AA₁ is

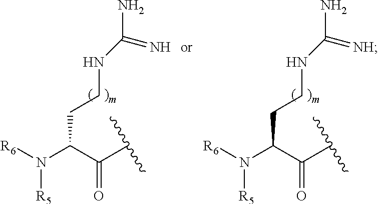

AA₂ is

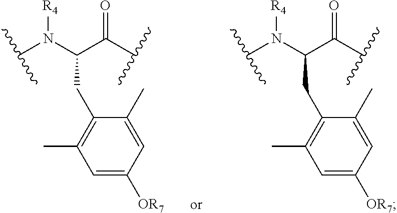

R₁ is

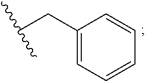 [SMILES: *Cc1ccccc1]

R^{2a} is

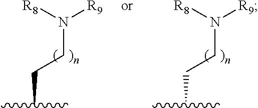

R₇ is H; and
X is

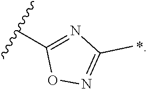 [SMILES: *c1nc(C)no1]

In some embodiments of the foregoing methods, uses, compositions formulations or medicaments, the peptidomimetic is a peptidomimetic of Formula II, Formula III, Formula IV, Formula V, Formula VI, Formula VII, Formula VIII, Formula IX, Formula X, Formula XI, Formula XII, Formula XIII, Formula XIV or Formula XV;

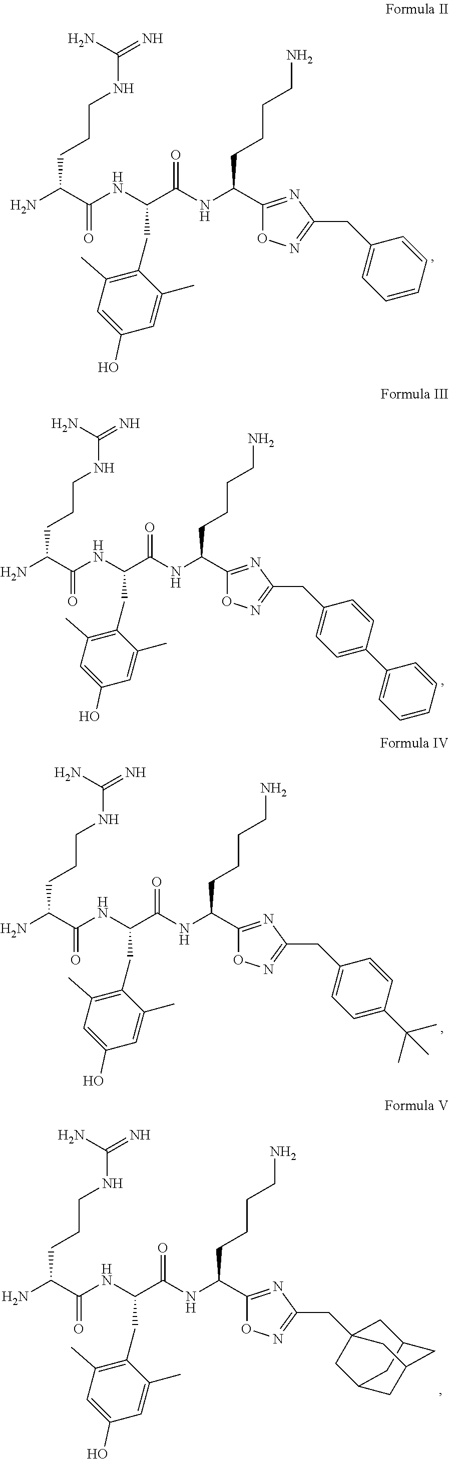 [SMILES: Cc1cc(O)cc(C)c1C[C@H](NC(=O)[C@H](N)CCCNC(=N)N)C(=O)N[C@@H](CCCCN)c1nc(Cc2ccccc2)no1 ; Cc1cc(O)cc(C)c1C[C@H](NC(=O)[C@H](N)CCCNC(=N)N)C(=O)N[C@@H](CCCCN)c1nc(Cc2ccc(-c3ccccc3)cc2)no1 ; Cc1cc(O)cc(C)c1C[C@H](NC(=O)[C@H](N)CCCNC(=N)N)C(=O)N[C@@H](CCCCN)c1nc(Cc2ccc(C(C)(C)C)cc2)no1 ; Cc1cc(O)cc(C)c1C[C@H](NC(=O)[C@H](N)CCCNC(=N)N)C(=O)N[C@@H](CCCCN)c1nc(CC23CC4CC(CC(C4)C2)C3)no1]

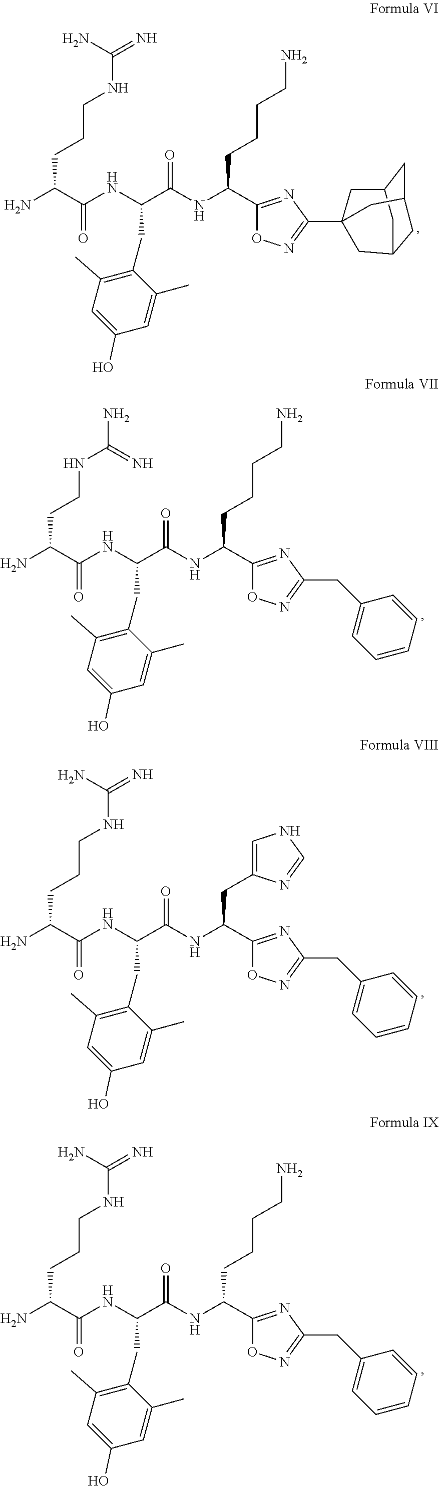

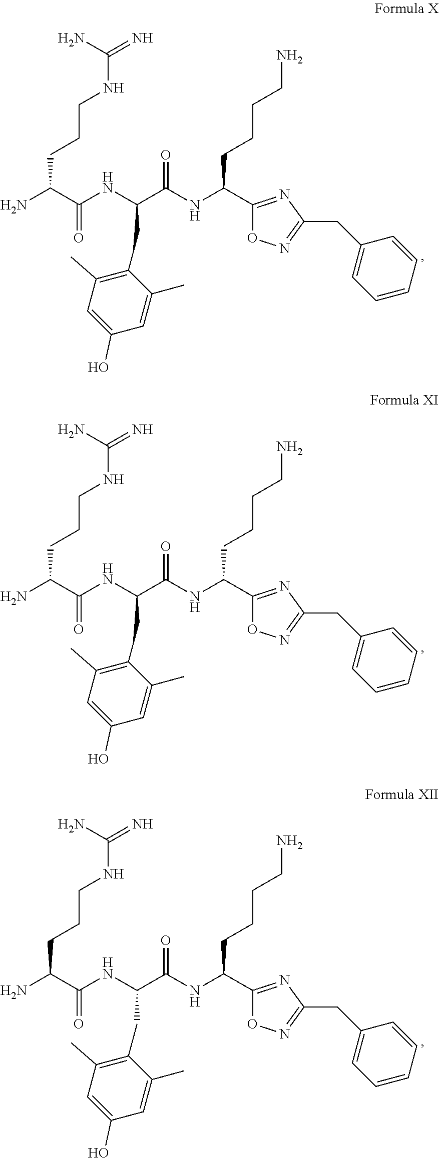 [SMILES: Cc1cc(O)cc(C)c1C[C@@H](NC(=O)[C@H](N)CCCNC(=N)N)C(=O)N[C@@H](CCCCN)c1nc(Cc2ccccc2)no1 ; Cc1cc(O)cc(C)c1C[C@@H](NC(=O)[C@H](N)CCCNC(=N)N)C(=O)N[C@H](CCCCN)c1nc(Cc2ccccc2)no1 ; Cc1cc(O)cc(C)c1C[C@H](NC(=O)[C@@H](N)CCCNC(=N)N)C(=O)N[C@@H](CCCCN)c1nc(Cc2ccccc2)no1]

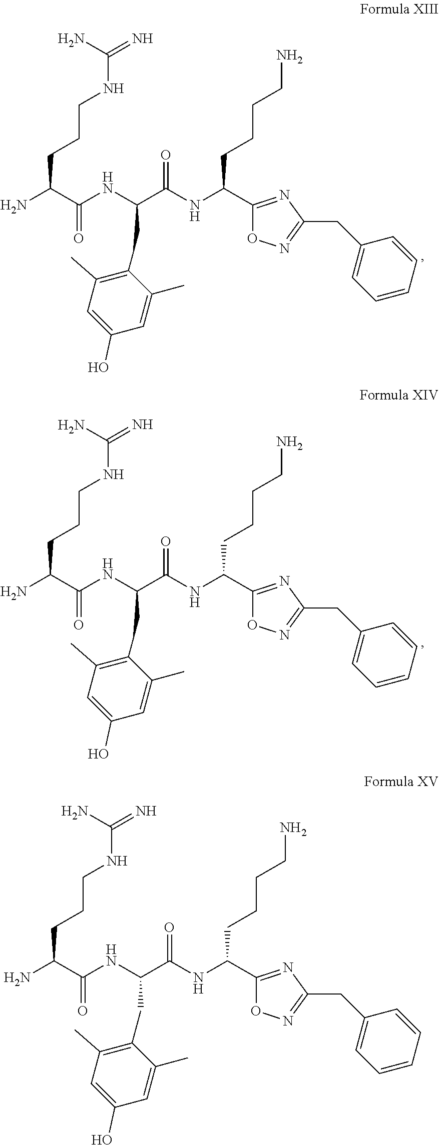 [SMILES: Cc1cc(O)cc(C)c1C[C@@H](NC(=O)[C@@H](N)CCCNC(=N)N)C(=O)N[C@@H](CCCCN)c1nc(Cc2ccccc2)no1 ; Cc1cc(O)cc(C)c1C[C@@H](NC(=O)[C@@H](N)CCCNC(=N)N)C(=O)N[C@H](CCCCN)c1nc(Cc2ccccc2)no1 ; Cc1cc(O)cc(C)c1C[C@H](NC(=O)[C@@H](N)CCCNC(=N)N)C(=O)N[C@H](CCCCN)c1nc(Cc2ccccc2)no1]

or a pharmaceutically acceptable salt, tautomer, hydrate, and/or solvate thereof and wherein one or more of the hydrogen atoms of the peptidomimetic is optionally substituted with a deuterium or fluorine atom.

In some embodiments of the foregoing methods, uses, compositions formulations or medicaments, the ophthalmic disease, disorder or condition is selected from the group consisting of: macular degeneration (including age-related macular degeneration), dry eye, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (e.g., Kearns-Sayre syndrome), and/or Leber congenital amaurosis (LCA).

In some embodiments of the foregoing methods, uses, compositions, formulations or medicaments, the subject is a human. In the practice of some of the foregoing methods, the subject has been diagnosed as having age-related macular degeneration (AMD). In the practice of some of the foregoing methods, the subject has drusen. In the practice of some of the foregoing methods, the subject has been diagnosed with geometric atrophy (GA). In the practice of some of the foregoing methods, the subject has been diagnosed with glaucoma.

In some embodiments of the foregoing methods, uses, compositions, formulations or medicaments, the peptidomimetic composition, formulation or medicament is administered orally. In some embodiments of the foregoing methods, uses, compositions, formulations or medicaments, the peptidomimetic composition, formulation or medicament is administered subcutaneously. In some embodiments of the foregoing methods, uses, compositions, formulations or medicaments, the peptidomimetic composition, formulation or medicament is administered topically. In some embodiments of the foregoing methods, uses, compositions, formulations or medicaments, the peptidomimetic composition, formulation or medicament is administered intraocularly. In some embodiments of the foregoing methods, uses, compositions, formulations or medicaments, the peptidomimetic composition, formulation or medicament is administered ophthalmically. In some embodiments of the foregoing methods, uses, compositions, formulations or medicaments, the peptidomimetic composition, formulation or medicament is administered intranasally. In some embodiments of the foregoing methods, uses, compositions, formulations or medicaments, the peptidomimetic composition, formulation or medicament is administered systemically. In some embodiments of the foregoing methods, uses, compositions, formulations or medicaments, the peptidomimetic composition, formulation or medicament is administered intravenously. In some embodiments of the foregoing methods, uses, compositions, formulations or medicaments, the peptidomimetic composition, formulation or medicament is administered intraperitoneally. In some embodiments of the foregoing methods, uses, compositions, formulations or medicaments, the peptidomimetic composition, formulation or medicament is administered intradermally. In some embodiments of the foregoing methods, uses, compositions, formulations or medicaments, the peptidomimetic composition, formulation or medicament is administered intrathecally. In some embodiments of the foregoing methods, uses, compositions, formulations or medicaments, the peptidomimetic composition, formulation or medicament is administered intracerebroventricularly. In some embodiments of the foregoing methods, uses, compositions, formulations or medicaments, the peptidomimetic composition, formulation or medicament is administered iontophoretically. In some embodiments of the foregoing methods, uses, compositions, formulations or medicaments, the peptidomimetic composition, formulation or medicament is administered transmucosally. In some embodiments of the foregoing methods, uses, compositions, formulations or medicaments, the peptidomimetic composition, formulation or medicament is administered intravitreally. In some embodiments of the foregoing methods, uses, compositions, formulations or medicaments, the peptidomimetic composition, formulation or medicament is administered intramuscularly. In some embodiments of the foregoing methods, uses, compositions, formulations or medicaments, the peptidomimetic composition, formulation or medicament is administered topically. In some embodiments of the foregoing methods, uses, compositions, formulations or medicaments, the peptidomimetic composition, formulation or medicament is administered intraocularly. In some embodiments of the foregoing methods, uses, compositions, formulations or medicaments, the peptidomimetic composition, formulation or medicament is administered ophthalmically. In some embodiments of the foregoing methods, uses, compositions, formulations or medicaments, the peptidomimetic, composition, formulation or medicament is administered daily for 2 weeks or more, 12 weeks or more, 24 weeks or more, 52 weeks or more, or 2 years or more.

In some embodiments, practice of the methods disclosed herein can further comprise administration an additional therapeutic agent (in addition to the one or more peptidomimetics). Said additional therapeutic agent can, for example, be selected from the group consisting of: an antioxidant, a metal complexer, an anti-inflammatory drug, an antibiotic, and an antihistamine. In one embodiment, the antioxidant is vitamin A, vitamin C, vitamin E, lycopene, selenium, α-lipoic acid, coenzyme Q, glutathione, or a carotenoid. In one embodiment, practice of the methods can further comprise administration of an additional therapeutic agent selected from the group consisting of: α-lipoic acid, aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, chrysoeriol, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, humanin, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, metformin, methazolamide, metipranolol, moxifloxacin, naphazoline, natamycin, necrostatins nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, PU-61, ranibizumab, resveratrol, rimexolone, scopolamine, sezolamide, squalamine, sulfacetamide, suprofen, tetracaine, tetracyclin, tetrahydrozoline, tetryzoline, timolol, tobramycin, TPP-Niacin, travoprost, triamcinulone, trifluoromethazolamide, trifluridine, trimethoprim, tropicamide, unoprostone, vidarbine, xylometazoline, ZLN005, pharmaceutically acceptable salts thereof, and combinations of two or more of the foregoing. In some embodiments, additional therapeutic agents can include, but are not limited to, administration of carbachiol (Carbastat® or Carboptic®), Polocarpine (Salagen®), timolol (Timoptic®), betaxolol (Betoptic® or Keflone®), Carteolol (Cartrol® or Ocupress®), Levobunolol (Liquifilm®), brimonidine (Lumify® or Mirvaso®), apraclonidine (Iopidine®), latanoprost (Xalantan®), travoprost (Travatan®), bimatoprost (Lumigan®), talfluprost (Taflotan®), unoprostone isopropyl (Rescula®), dorzolamide (Trusopt®), brinzolamide (Azopt®), acetazolamide (Diamox®), methazolamide (Neptazane®), brimonidine tartrate/timolol maleate (Combigan®), timolo-dorzolamide (Cosopt®), travoprost-timolol (DuoTrav®) and/or latanoprost and timolol maleate (Xalacom®).

In some embodiments, a composition, formulation or medicament can further comprise an additional therapeutic agent. Said additional therapeutic agent can, for example, be selected from the group consisting of: an antioxidant, a metal complexer, an anti-inflammatory drug, an antibiotic, and an antihistamine. In one embodiment, the antioxidant is vitamin A, vitamin C, vitamin E, lycopene, selenium, α-lipoic acid, coenzyme Q, glutathione, or a carotenoid. In one embodiment, practice of the methods can further comprise administration of an additional therapeutic agent selected from the group consisting of: α-lipoic acid, aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, chrysoeriol, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, humanin, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, metformin, methazolamide, metipranolol, moxifloxacin, naphazoline, natamycin, necrostatins nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, PU-61, ranibizumab, resveratrol, rimexolone, scopolamine, sezolamide, squalamine, sulfacetamide, suprofen, tetracaine, tetracyclin, tetrahydrozoline, tetryzoline, timolol, tobramycin, TPP-Niacin, travoprost, triamcinulone, trifluoromethazolamide, trifluridine, trimethoprim, tropicamide, unoprostone, vidarbine, xylometazoline, ZLN005, pharmaceutically acceptable salts thereof, and combinations of two or more of the foregoing. In some embodiments, additional therapeutic agents can include, but are not limited to, administration of carbachiol (Carbastat® or Carboptic®), Polocarpine (Salagen®), timolol (Timoptic®), betaxolol (Betoptic® or Keflone®), Carteolol (Cartrol® or Ocupress®), Levobunolol (Liquifilm®), brimonidine (Lumify® or Mirvaso®), apraclonidine (Iopidine®), latanoprost (Xalantan®), travoprost (Travatan®), bimatoprost (Lumigan®), talfluprost (Taflotan®), unoprostone isopropyl (Rescula®), dorzolamide (Trusopt®), brinzolamide (Azopt®), acetazolamide (Diamox®), methazolamide (Neptazane®), brimonidine tartrate/timolol maleate (Combigan®), timolo-dorzolamide (Cosopt®), travoprost-timolol (DuoTrav®) and/or latanoprost and timolol maleate (Xalacom®).

### BRIEF DESCRIPTION OF THE FIGURES

FIG. 1A is a graph of data comparing the concentration of either elamipretide or compound of Formula IIa in rabbit plasma at various time points following subcutaneous (SC) injection. FIG. 1B is a graph of data comparing the concentration of either elamipretide or compound of Formula IIa in rabbit plasma at various time points following 5 days of twice daily topical administration of eye drops.

FIG. 2A is a graph of data comparing the concentration of either elamipretide or compound of Formula IIa in the retina of a rabbit at various time points following subcutaneous (SC) injection. FIG. 2B is a graph of data comparing the concentration of either elamipretide or compound of Formula IIa in the retina of a rabbit at various time points following 5 days of twice daily topical administration of eye drops.

FIG. 3 is a graph of data comparing the concentration of either elamipretide or compound of Formula IIa in the conjunctiva of a rabbit at various time points following 5 days of twice daily topical administration of eye drops.

FIG. 4 is a graph of data comparing the concentration of either elamipretide or compound of Formula IIa in the cornea of a rabbit at various time points following 5 days of twice daily topical administration of eye drops.

FIG. 5 is a graph of data comparing the concentration of either elamipretide or compound of Formula IIa in the aqueous humor of a rabbit at various time points following 5 days of twice daily topical administration of eye drops.

FIG. 6 is a graph of data comparing the concentration of either elamipretide or compound of Formula IIa in the sclera of a rabbit at various time points following 5 days of twice daily topical administration of eye drops.

FIG. 7 is a graph of data comparing the concentration of either elamipretide or compound of Formula IIa in the optical nerve head of a rabbit at various time points following 5 days of twice daily topical administration of eye drops.

FIG. 8A is a schematic overview of the experimental approach using nitrite modification of extracellular matrix (ECM) as a model of an aged Bruch's membrane. RPE cells=retinal pigment epithelial cells.

FIGS. 8B-8G are images showing the differentiation of human-induced pluripotent stem cell (iPSC)-derived retinal pigment epithelial (RPE) cells from donor fibroblasts. Fibroblasts (FIG. 8B) were reprogrammed into an undifferentiated human iPSC colony (FIG. 8C). iPSCs were induced to become embryoid bodies (EBs) in a floating culture (FIG. 8D). Induction of neural rosettes by day 14 post-differentiation (FIG. 8E), and a pigmented monolayer of iPSC-derived RPE cells formed by day 45 post-differentiation (FIGS. 8F and 8G).

FIG. 8H are images showing that after differentiation, iPSC-derived RPE cell lines from age-related macular degeneration (AMD) donors stained positive for ZO-1, NA-K ATPase, and RPE65. Nuclei stained with DAPI. Scale bar=20 μm.

FIG. 8I is an image of pigmented iPSC-derived RPE cells.

FIG. 8J is a chart showing the effect of elamipretide (309) and compound of Formula IIa (146c) on iPSC-derived RPE cell viability on nitrite-modified ECM. *p<0.05.

FIG. 8K is a heat map showing hierarchical cluster analysis (HCA) of AMD-derived RPE cells cultured on nitrite-modified ECM versus AMD-derived RPE cultured on unmodified ECM.

FIGS. 8L-8T are charts showing the effects of elamipretide (309) and compound of Formula IIa (146c) on complement-related gene expression in AMD-derived RPE cells cultured on an in vitro Bruch's membrane model. From left to right on each chart, the following groups are shown: AMD-unmodified; AMD nitrite; AMD nitrite 146c 10 nM; AMD nitrite 146c 100 nM; AMD nitrite 146c 1000 nM; AMD nitrite 309 10 nM; AMD nitrite 309 100 nM; and AMD nitrite 309 1000 nM.

FIG. 8U is a heat map showing HCA of 13 mitochondrial encoded genes in AMD-derived RPE cells cultured on nitrite-modified versus unmodified ECM.

FIG. 8V is a heat map showing HCA of 293 mitochondrial-related genes in AMD-derived RPE cells cultured on nitrite-modified versus unmodified ECM.

FIGS. 8W-8Z are charts showing the effects of elamipretide (309) and compound of Formula IIa (146c) on gene expression levels from mitochondrial-related genes. From left to right on each chart, the following groups are shown: AMD-unmodified; AMD nitrite; AMD nitrite 146c 10 nM; AMD nitrite 146c 100 nM; AMD nitrite 146c 1000 nM; AMD nitrite 309 10 nM; AMD nitrite 309 100 nM; and AMD nitrite 309 1000 nM.

FIGS. 8AA-8AL are charts showing the effects of elamipretide (309) and compound of Formula IIa (146c) on mitochondrial function in patient-derived RPE cells: ATP production (FIGS. 8AA-8AC); basal respiration (FIGS. 8AD-8AF); maximal respiration (FIGS. 8AG-8AI); and spare respiratory capacity (FIGS. 8AJ-8AL).

FIG. 9 is a diagram showing the daily rotation of injection sites described in Example 4.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, NY, 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

### Definitions

The definitions of certain terms as used in this specification are provided below. Unless defined otherwise, all technical and scientific terms used herein generally have the same meaning as commonly understood by one of ordinary skill in the art to which the present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent, drug, therapeutic agent, peptide or peptidomimetic to a subject includes any route of introducing or delivering to a subject a compound, composition or formulation to perform its intended function. Administration can be carried out by any suitable route, such as oral administration. Administration can be carried out subcutaneously. Administration can be carried out intravitreally. Administration can be carried out topically. Administration can be carried out intraocularly. Administration can be carried out ophthalmically. Administration can be carried out systemically. Alternatively, administration may be carried out intranasally, intravenously, intraperitoneally, intradermally, intrathecally, intracerebroventricularly, iontophoretically, transmucosally or intramuscularly. Administration includes self-administration and the administration by another.

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject

As used herein, the term “amino acid” includes naturally-occurring amino acids and synthetic amino acids, as well as amino acid analogs and amino acid mimetics that function in a manner similar to the naturally-occurring amino acids. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, nor leucine, methionine sulfoxide, methionine methyl sulfonium. Such analogs have modified R groups (e.g., nor leucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

The term “DMT”, “Dmt”, “2′,6′-DMT” or “2′,6′-Dmt” or refers to 2,6-di(methyl)tyrosine (e.g., 2,6-dimethyl-L-tyrosine; CAS 123715-02-6).

As used herein, the phrase “delaying the onset of” refers to, in a statistical sample, postponing, hindering the occurrence of a disease, disorder or condition, or causing one or more signs, symptoms or conditions of a disease, disorder or condition to occur more slowly than normal, in a sample or subject administered a therapeutic agent relative to a control sample or subject.

As used herein, the term “effective amount” refers to a quantity sufficient to achieve a desired therapeutic and/or prophylactic effect, e.g., an amount which results in the prevention of, a decrease in, or delay in the onset of the symptoms associated with an ophthalmic condition. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic agents/compounds. In the methods described herein, the peptidomimetics may be administered to a subject having one or more signs or symptoms of an ophthalmic condition. For example, a “therapeutically effective amount” of the peptidomimetics is meant levels in which the physiological effects of an ophthalmic condition are, at a minimum, ameliorated or delayed in progression and/or severity.

As used herein, the term “hydrate” refers to a compound which is associated with water. The number of the water molecules contained in a hydrate of a compound may be (or may not be) in a definite ratio to the number of the compound molecules in the hydrate.

As used herein, “inhibit” or “inhibiting” refers to the reduction in a sign, symptom or condition (e.g. risk factor) associated with a disease, disorder or condition by an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject)

As used herein, the terms “peptidomimetic” refers to a small peptide-like polymer comprising two or more amino acids but that also contains a non-peptide-like modification. A peptidomimetic can arise either by modification of an existing peptide, or by designing similar molecules that mimic peptide function. In some embodiments, a peptidomimetic has the Formula I, II, IIa, III, IV, V, VI, VII, VIII, IX, X, XI, XII, XIII, XIV or XV, or a pharmaceutically acceptable salt, tautomer, hydrate, and/or solvate thereof, as defined herein.

As used herein, “prevention” or “preventing” of a disease, disorder, or condition refers to results that, in a statistical sample, exhibit a reduction in the occurrence of the disease, disorder, or condition in a sample or subject administered a therapeutic agent relative to a control sample or subject, or exhibit a delay in the onset of one or more symptoms of the disease, disorder, or condition relative to the control sample or subject. Such prevention is sometimes referred to as a prophylactic treatment.

The terms “pharmaceutically acceptable carrier” and “carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E.W. Martin, herein incorporated by reference in its entirety

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

In the context of therapeutic use or administration, the term “separate” or “separately” refers to an administration of at least two active ingredients by different routes, formulations, and/or pharmaceutical compositions.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “solvate” refers to forms of a compound (e.g., peptide or peptidomimetic) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like

As used herein, the terms “subject” and “patient” are used interchangeably.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating ALS, α-synucleinopathies, or TDP-43 proteinopathies, resulting in increased therapeutic efficacy and decreased side-effects.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “treating” or “treatment” or “alleviation” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

As used herein, the terms “(R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide,” “(D-Arg-DMT-NH((S)-5-amino-1-(3-benzyl-1,2,4-oxadiazol-5-yl)pent-1-yl),”, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, “compound 7a,” and “7a” refer to the same peptidomimetic, are used interchangeably herein, and refer to a compound of the following Formula II:

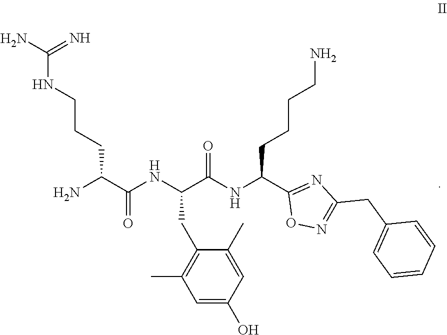 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

The term “(R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide,”, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, “(D-Arg-DMT-NH((S)-5-amino-1-(3-benzyl-1,2,4-oxadiazol-5-yl)pent-1-yl),” “compound 7a,” and “7a” is intended to include pharmaceutically acceptable salt forms thereof such as the tri- (or tris)-HCl salt of Formula IIa:

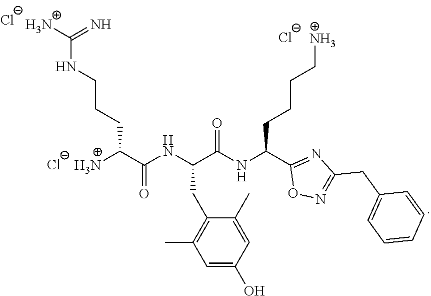 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

### Peptidomimetics:

In some embodiments, the present disclosure provides a compound of Formula I, or a pharmaceutically acceptable salt, tautomer, hydrate, and/or solvate thereof:

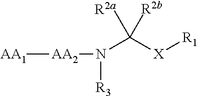

wherein
AA₁ is selected from

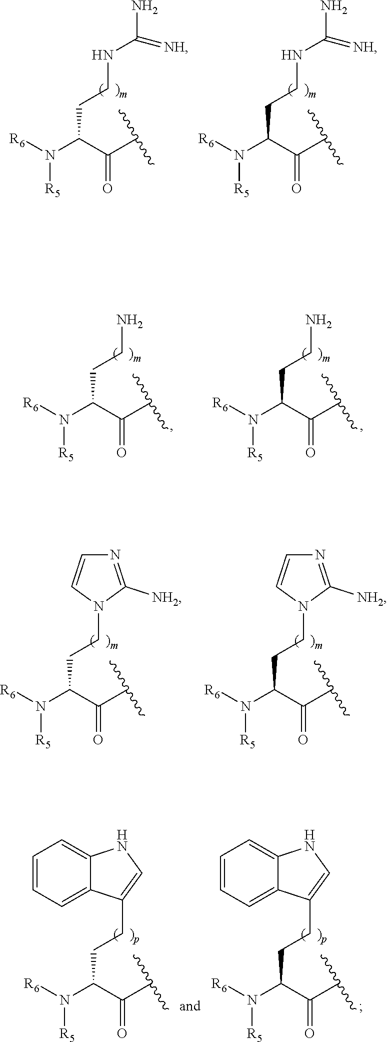

AA₂ is selected from

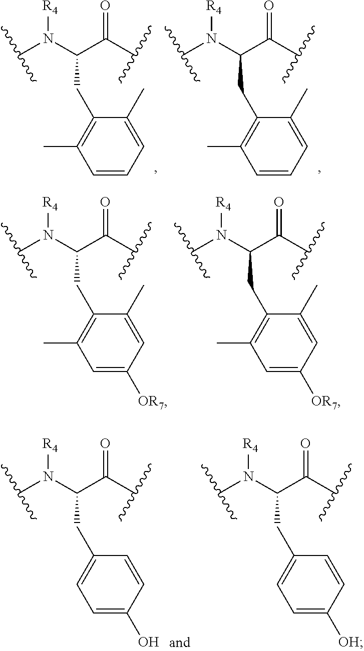

R₁ is selected from

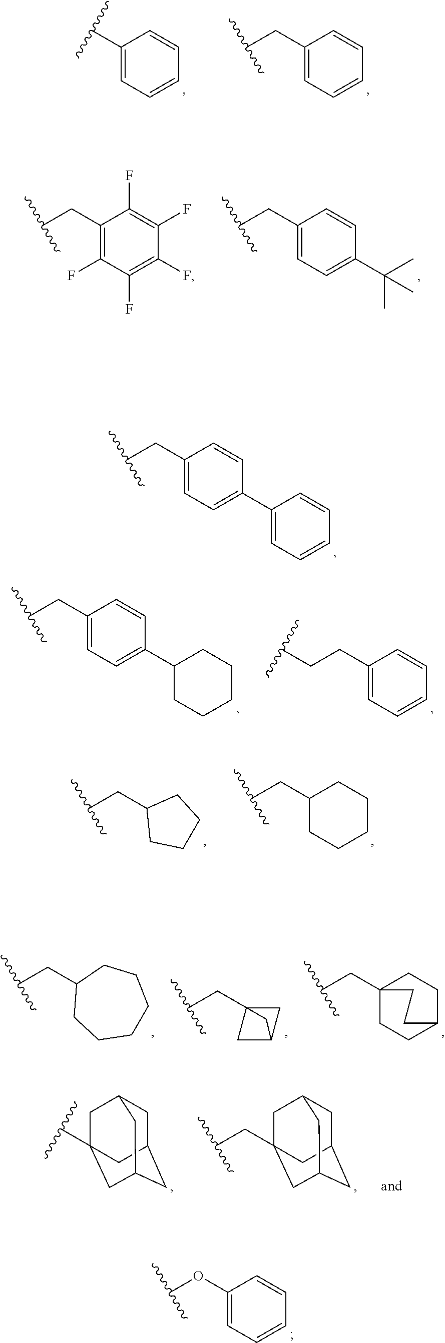

R^{2a} is selected from

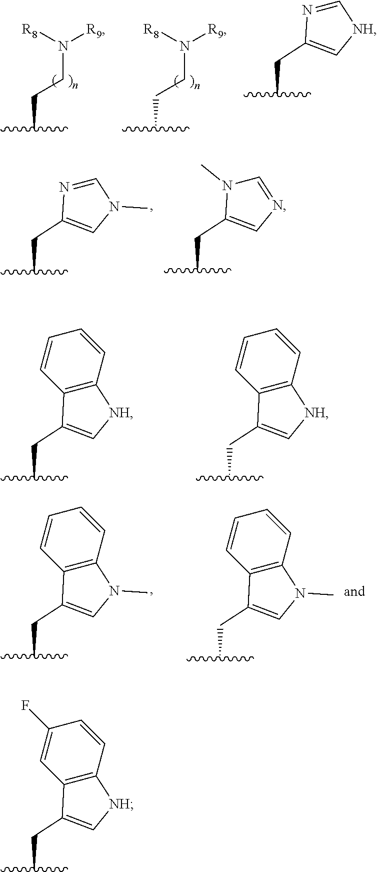

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

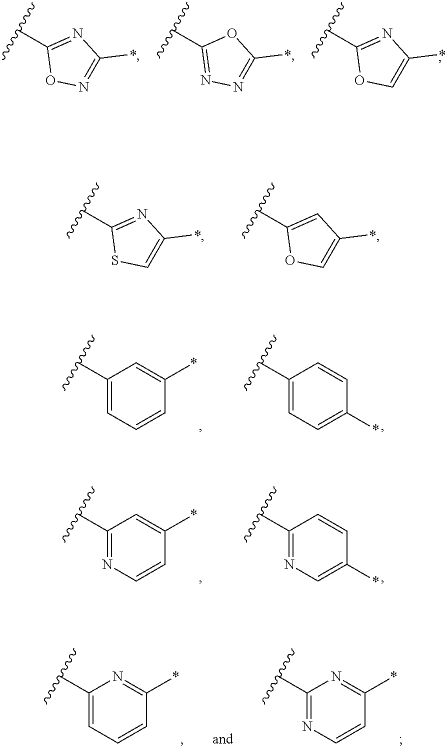

and
* denotes the point of attachment of X to R₁, and wherein one or more of the hydrogen atoms of the peptidomimetic is optionally substituted with a deuterium or fluorine atom.

In some embodiments, AA₁ is

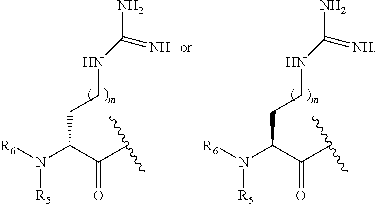

In some embodiments, AA₁ is

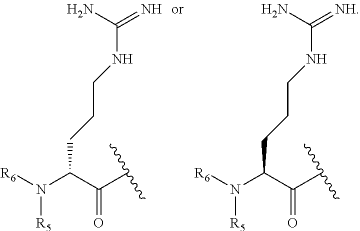

In some embodiments, AA₁ is

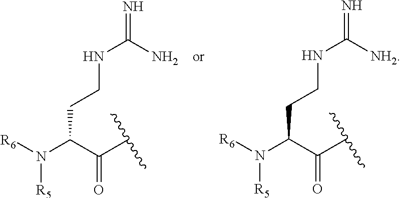

In some embodiments, AA₁ is

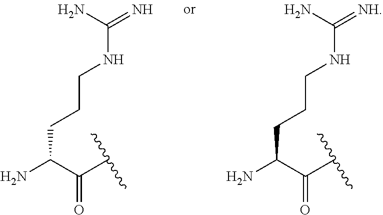 [SMILES: *C(=O)[C@H](N)CCCNC(=N)N ; *C(=O)[C@@H](N)CCCNC(=N)N]

In some embodiments, AA₁ is

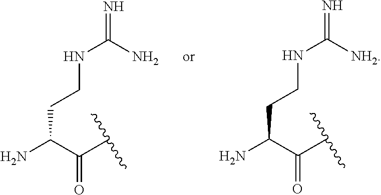 [SMILES: *C(=O)[C@H](N)CCNC(=N)N ; *C(=O)[C@@H](N)CCNC(=N)N]

In some embodiments, AA₁ is

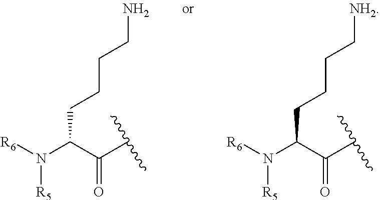

In some embodiments, AA₁ is

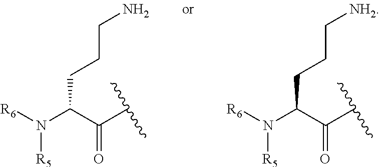

In some embodiments, AA₁ is

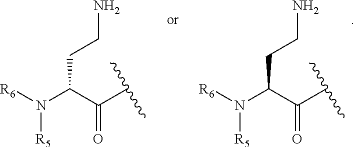

In some embodiments, AA₁ is

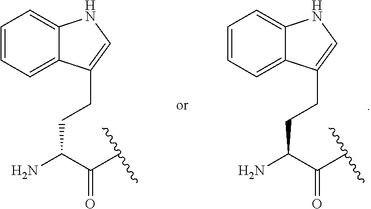 [SMILES: *C(=O)[C@H](N)CCc1c[nH]c2ccccc12 ; *C(=O)[C@@H](N)CCc1c[nH]c2ccccc12]

In some embodiments, AA₂ is

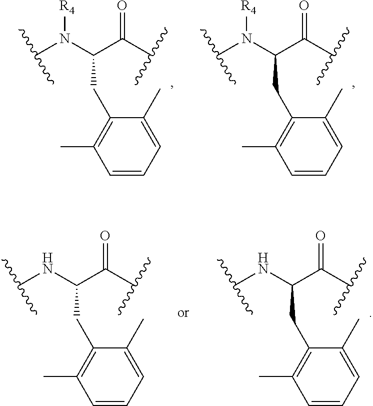

In some embodiments, AA₂ is

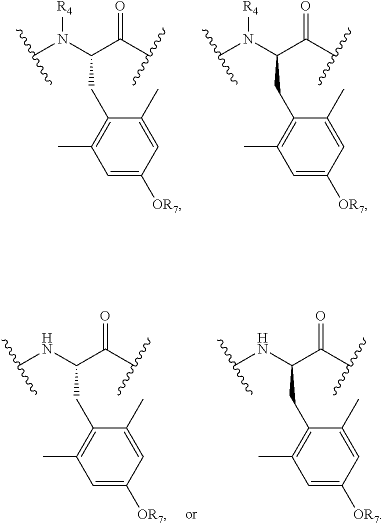

In some embodiments, AA₂ is

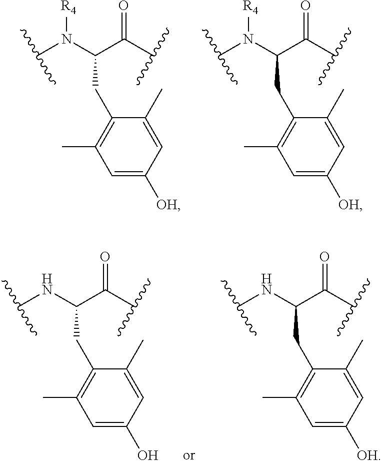

In some embodiments, R₁ is

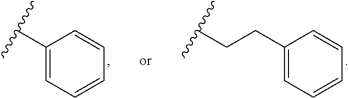 [SMILES: *c1ccccc1 ; *CCc1ccccc1]

In some embodiments, R₁ is

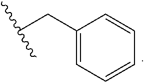 [SMILES: *Cc1ccccc1]

In some embodiments, R₁ is

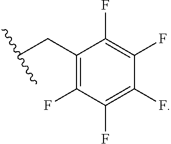 [SMILES: *Cc1c(F)c(F)c(F)c(F)c1F]

In some embodiments, R₁ is

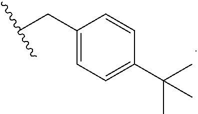 [SMILES: *Cc1ccc(C(C)(C)C)cc1]

In some embodiments, R₁ is

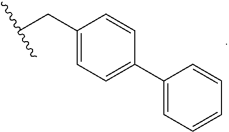 [SMILES: *Cc1ccc(-c2ccccc2)cc1]

In some embodiments, R₁ is

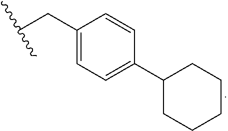 [SMILES: *Cc1ccc(C2CCCCC2)cc1]

In some embodiments R₁ is

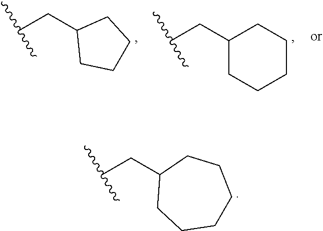

In some embodiments, R₁ is

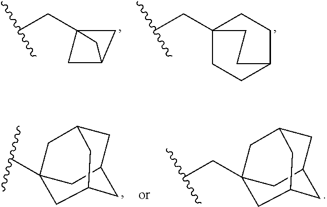

In some embodiments, R₁ is

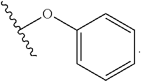 [SMILES: *Oc1ccccc1]

In some embodiments, R₁ is

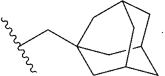 [SMILES: *CC12CC3CC(CC(C3)C1)C2]

In some embodiments, R^{2a} is

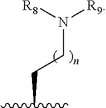

In some embodiments R^{2a} is

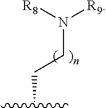

In some embodiments, R^{2a} is

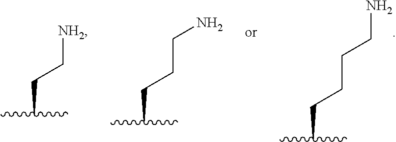

In some embodiments, R^{2a} is

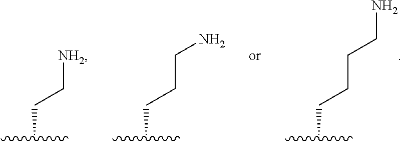

In some embodiments, R^{2a} is

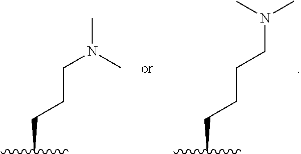

In some embodiments, R^{2a} is

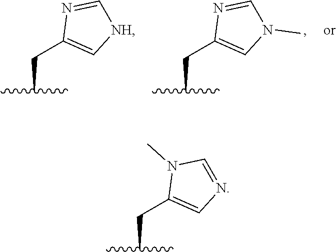

In some embodiments, R^{2a} is

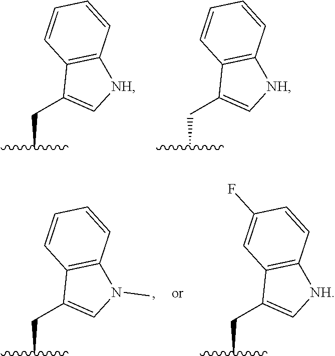

In some embodiments, R^{2b} is H. In some embodiments, R^{2b} is methyl.

In some embodiments, R₃ is H. In some embodiments, R₃ is (C₁-C₆)alkyl. In some embodiments, R₃ is methyl, ethyl, n-propyl, i-propyl, n-butyl, i-butyl, or t-butyl. In some embodiments, R₃ is methyl. In some embodiments, R₃ is ethyl.

In some embodiments, R₄ is H. In some embodiments, R₄ is (C₁-C₆)alkyl. In some embodiments, R₄ is methyl, ethyl, n-propyl, i-propyl, n-butyl, i-butyl, or t-butyl. In some embodiments, R₄ is methyl. In some embodiments, R₄ is ethyl.

In some embodiments, R₃ and R₄ are the same. In some embodiments, R₃ and R₄ are different.

In some embodiments, R₅ is H. In some embodiments, R₅ is methyl.

In some embodiments, R₆ is H. In some embodiments, R₆ is methyl.

In some embodiments, R₅ and R₆ are the same. In some embodiments, R₅ and R₆ are different.

In some embodiments, R₅ and R₆ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, R₇ is H. In some embodiments, R₇ is (C₁-C₆)alkyl. In some embodiments, R₇ is methyl, ethyl, n-propyl, i-propyl, n-butyl, i-butyl, or t-butyl. In some embodiments, R₇ is methyl.

In some embodiments, R₇ is cycloalkyl. In some embodiments, R₇ is cyclopropyl, cyclobutyl, cyclopropyl, or cyclohexyl. In some embodiments, R₇ is aryl. In some embodiments, R₇ is phenyl.

In some embodiments, R₈ is H. In some embodiments, R₈ is (C₁-C₆)alkyl. In some embodiments, R₈ is methyl, ethyl, n-propyl, i-propyl, n-butyl, i-butyl, or t-butyl. In some embodiments, R₈ is methyl. In some embodiments, R₈ is ethyl.

In some embodiments, R₈ is cycloalkyl. In some embodiments, R₈ is cyclopropyl, cyclobutyl, cyclopropyl, or cyclohexyl. In some embodiments, R₈ is aryl. In some embodiments, R₈ is phenyl.

In some embodiments, R₉ is H. In some embodiments, R₉ is (C₁-C₆)alkyl. In some embodiments, R₉ is methyl, ethyl, n-propyl, i-propyl, n-butyl, i-butyl, or t-butyl. In some embodiments, R₉ is methyl. In some embodiments, R₉ is ethyl.

In some embodiments, R₉ is cycloalkyl. In some embodiments, R₉ is cyclopropyl, cyclobutyl, cyclopropyl, or cyclohexyl. In some embodiments, R₉ is aryl. In some embodiments, R₉ is phenyl.

In some embodiments, R₈ and R₉ are the same. In some embodiments, R₈ and R₉ are different.

In some embodiments, R₈ and R₉ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, X is

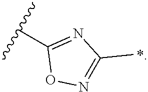 [SMILES: *c1nc(C)no1]

In some embodiments, X is

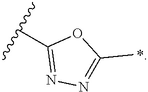 [SMILES: *c1nnc(C)o1]

In some embodiments, X is

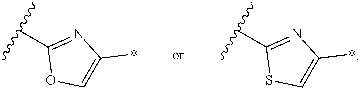

In some embodiments, X is

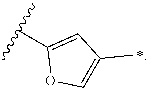 [SMILES: *c1cc(C)co1]

In some embodiments, X is

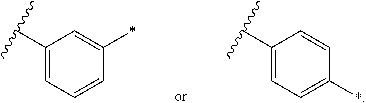

In some embodiments, X is

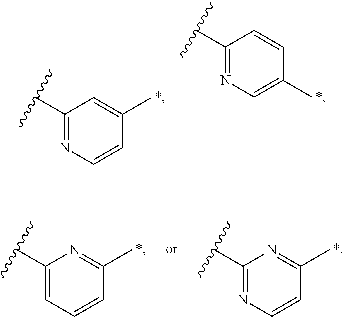

In some embodiments, n is 1. In some embodiments, n is 2. In some embodiments, n is 3. In some embodiments, m is 1. In some embodiments, m is 2. In some embodiments, m is 3. In some embodiments p is 0. In some embodiments, p is 1.

In some embodiments, AA₁ is selected from

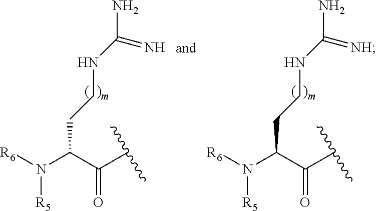

AA₂ is selected from

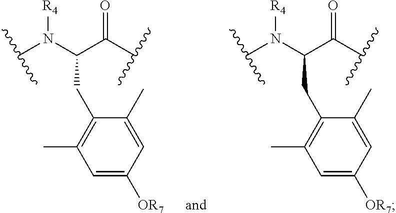

R₁ is selected from

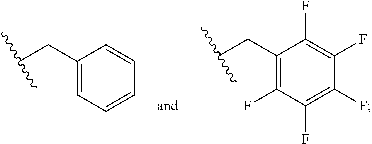 [SMILES: *Cc1ccccc1 ; *Cc1c(F)c(F)c(F)c(F)c1F]

R^{2a} is selected from

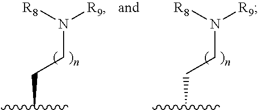

R^{2b} is H; R₃ and R₄ are independently selected from H and methyl; R₅ and R₆ are independently H or methyl; R₇ is selected from H and methyl; R₈ and R₉ are independently selected from H and methyl; and X is selected from

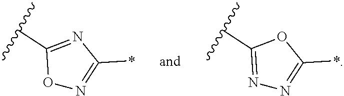

In some embodiments, AA₁ is

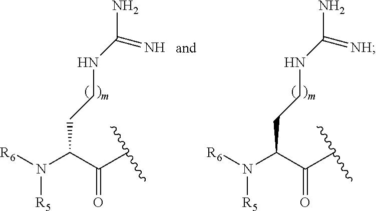

### AA₂ is

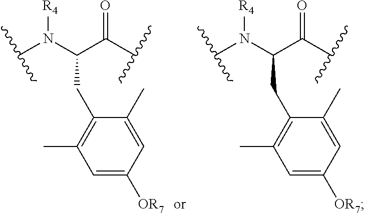

### R₁ is

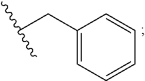 [SMILES: *Cc1ccccc1]

### R^{2a} is

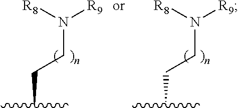

### R₇ is H; and X is

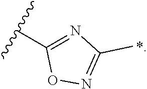 [SMILES: *c1nc(C)no1]

In some embodiments, the peptidomimetic is a peptidomimetic of Formula II, Formula III, Formula IV, Formula V, Formula VI, Formula VII, Formula VIII, Formula IX, Formula X, Formula XI, Formula XII, Formula XIII, Formula XIV or Formula XV;

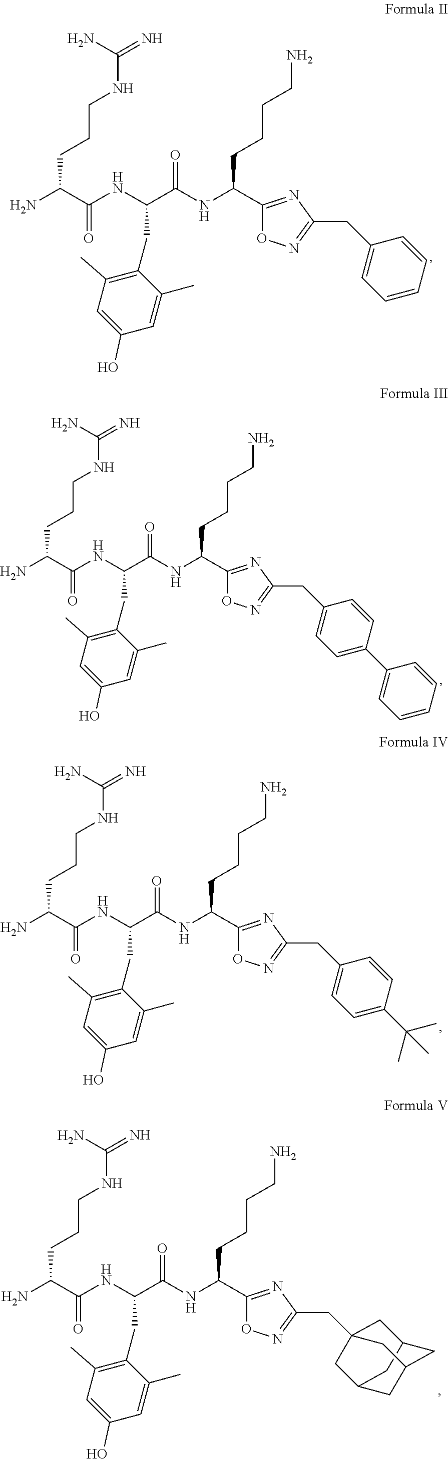 [SMILES: Cc1cc(O)cc(C)c1C[C@H](NC(=O)[C@H](N)CCCNC(=N)N)C(=O)N[C@@H](CCCCN)c1nc(Cc2ccccc2)no1 ; Cc1cc(O)cc(C)c1C[C@H](NC(=O)[C@H](N)CCCNC(=N)N)C(=O)N[C@@H](CCCCN)c1nc(Cc2ccc(-c3ccccc3)cc2)no1 ; Cc1cc(O)cc(C)c1C[C@H](NC(=O)[C@H](N)CCCNC(=N)N)C(=O)N[C@@H](CCCCN)c1nc(Cc2ccc(C(C)(C)C)cc2)no1 ; Cc1cc(O)cc(C)c1C[C@H](NC(=O)[C@H](N)CCCNC(=N)N)C(=O)N[C@@H](CCCCN)c1nc(CC23CC4CC(CC(C4)C2)C3)no1]

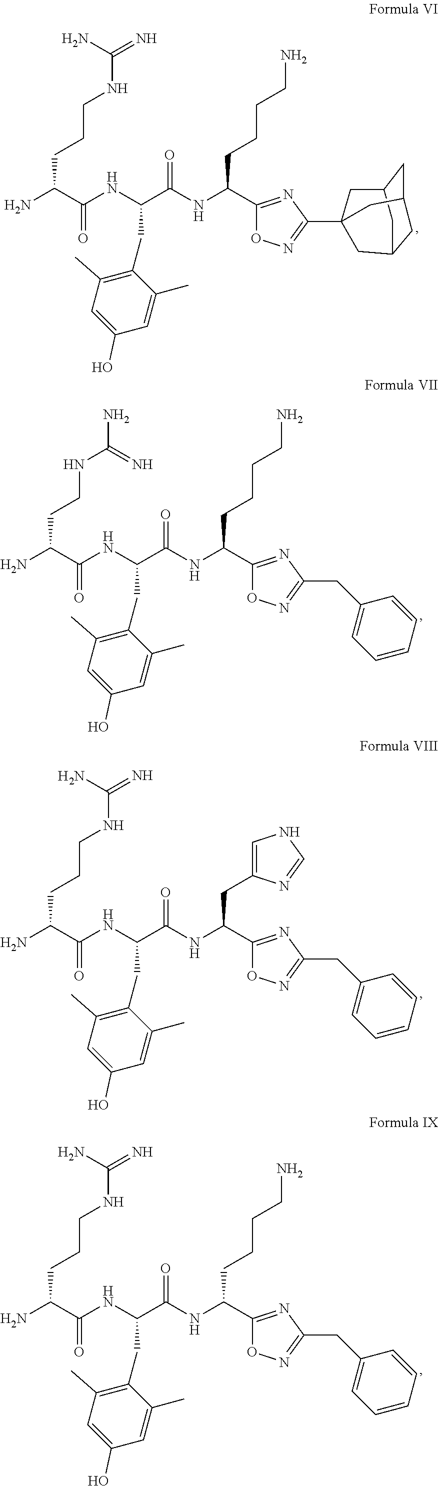 [SMILES: Cc1cc(O)cc(C)c1C[C@H](NC(=O)[C@H](N)CCCNC(=N)N)C(=O)N[C@@H](CCCCN)c1nc(C23CC4CC(CC(C4)C2)C3)no1 ; Cc1cc(O)cc(C)c1C[C@H](NC(=O)[C@H](N)CCNC(=N)N)C(=O)N[C@@H](CCCCN)c1nc(Cc2ccccc2)no1 ; Cc1cc(O)cc(C)c1C[C@H](NC(=O)[C@H](N)CCCNC(=N)N)C(=O)N[C@@H](Cc1c[nH]cn1)c1nc(Cc2ccccc2)no1 ; Cc1cc(O)cc(C)c1C[C@H](NC(=O)[C@H](N)CCCNC(=N)N)C(=O)N[C@H](CCCCN)c1nc(Cc2ccccc2)no1]

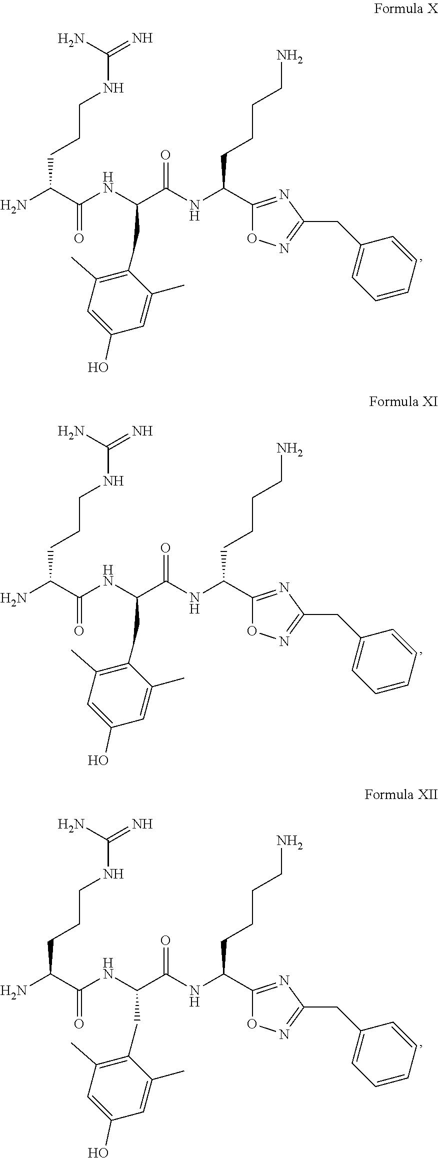 [SMILES: Cc1cc(O)cc(C)c1C[C@@H](NC(=O)[C@H](N)CCCNC(=N)N)C(=O)N[C@@H](CCCCN)c1nc(Cc2ccccc2)no1 ; Cc1cc(O)cc(C)c1C[C@@H](NC(=O)[C@H](N)CCCNC(=N)N)C(=O)N[C@H](CCCCN)c1nc(Cc2ccccc2)no1 ; Cc1cc(O)cc(C)c1C[C@H](NC(=O)[C@@H](N)CCCNC(=N)N)C(=O)N[C@@H](CCCCN)c1nc(Cc2ccccc2)no1]

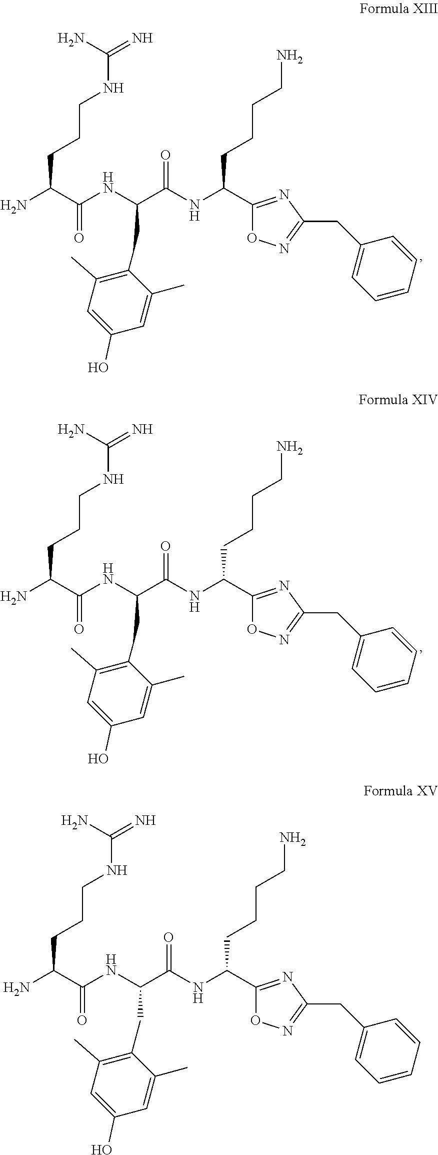 [SMILES: Cc1cc(O)cc(C)c1C[C@@H](NC(=O)[C@@H](N)CCCNC(=N)N)C(=O)N[C@@H](CCCCN)c1nc(Cc2ccccc2)no1 ; Cc1cc(O)cc(C)c1C[C@@H](NC(=O)[C@@H](N)CCCNC(=N)N)C(=O)N[C@H](CCCCN)c1nc(Cc2ccccc2)no1 ; Cc1cc(O)cc(C)c1C[C@H](NC(=O)[C@@H](N)CCCNC(=N)N)C(=O)N[C@H](CCCCN)c1nc(Cc2ccccc2)no1]

or a pharmaceutically acceptable salt, tautomer, hydrate, and/or solvate thereof and wherein one or more of the hydrogen atoms of the peptidomimetic is optionally substituted with a deuterium or fluorine atom.

In some embodiments, peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or a pharmaceutically acceptable salt (e.g. IIa), stereoisomer, tautomer, hydrate, and/or solvate thereof, and wherein one or more of the hydrogen atoms of the molecule is optionally substituted with a deuterium or fluorine atom.

The chiral centers of the peptidomimetic disclosed herein may be in either the R- or S-configuration as discussed in more detail below.

### Chiral/Stereochemistry Considerations:

Peptidomimetics described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The peptidomimetics additionally encompasses compounds described herein as individual isomers substantially free of other isomers, and alternatively, as mixtures of various isomers.

As used herein, a pure enantiomeric peptidomimetic is substantially free from other enantiomers or stereoisomers of the compound (i.e., in enantiomeric excess). In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that for a “D”-amino acid the configuration is “R” and for an “L”-amino acid, the configuration is “S” (with the exception of cysteine where the assignment is reversed because of the presence of sulfur in the side chain). In some embodiments, ‘substantially free,’ refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

The nomenclature used to define the peptide compounds described herein is that typically used in the art wherein the amino group at the N-terminus appears to the left and the carboxyl group at the C-terminus appears to the right, provided however that the peptidomimetics disclosed herein do not contain a carboxylic acid moiety or amide moiety at the C-terminus.

A capital letter “D” used in conjunction with an abbreviation for an amino acid residue refers to the D-form of the amino acid residue. For example, D-Arg is a commercially available D-amino acid.

The peptidomimetics disclosed herein can exist in unsolvated forms as well as solvated forms, including hydrated forms. Solvated forms can exist, for example, because it is difficult or impossible to remove all the solvent from the peptidomimetic post synthesis. In general, the solvated forms are equivalent to unsolvated forms and are encompassed within the scope of the present application. Certain peptidomimetics of the present application may exist in multiple crystalline or amorphous forms. Certain peptidomimetics of the present application may exist in various tautomeric forms. Certain peptidomimetics of the present application may exist in various salt forms. In general, all physical forms are equivalent for the uses contemplated by the present application and are intended to be within the scope of the present application.

In some embodiments, the peptidomimetics disclosed herein is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (II), or a pharmaceutically acceptable salt (e.g. IIa), stereoisomer, tautomer, hydrate, and/or solvate thereof and the subject has been diagnosed as having an ophthalmic condition or disease. In some embodiments of the peptidomimetics of the present technology, the treating or preventing comprises the treatment or prevention of macular degeneration (including age-related macular degeneration), dry eye, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (e.g., Kearns-Sayre syndrome), Leber congenital amaurosis (LCA), or in mammalian subjects. In some embodiments, the subject is human.

In some embodiments of the peptidomimetics of the present technology, the peptidomimetic is administered (neat or in a formulation or medicament) to the subject separately, sequentially, or simultaneously with an additional therapeutic agent or an additional therapeutic treatment. In some embodiments, the additional therapeutic agent is selected from the group consisting of: an antioxidant, a metal complexer, an anti-inflammatory drug, an antibiotic, and an antihistamine. In one embodiment, the antioxidant is vitamin A, vitamin C, vitamin E, lycopene, selenium, α-lipoic acid, coenzyme Q, glutathione, or a carotenoid. In one embodiment, the formulation further comprises an active agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations of two or more of the foregoing. In some embodiments, the additional therapeutic agents include, but are not limited to, administration of carbachiol (Carbastat® or Carboptic®), Polocarpine (Salagen®), timolol (Timoptic®), betaxolol (Betoptic® or Keflone®), Carteolol (Cartrol® or Ocupress®), Levobunolol (Liquifilm®), brimonidine (Lumify® or Mirvaso®), apraclonidine (Iopidine®), latanoprost (Xalantan®), travoprost (Travatan®), bimatoprost (Lumigan®), talfluprost (Taflotan®), unoprostone isopropyl (Rescula®), dorzolamide (Trusopt®), brinzolamide (Azopt®), acetazolamide (Diamox®), methazolamide (Neptazane®), brimonidine tartrate/timolol maleate (Combigan®), timolo-dorzolamide (Cosopt®), travoprost-timolol (DuoTrav®) and latanoprost and timolol maleate (Xalacom®).

### Synthesis of Peptidomimetics:

The peptidomimetic compounds of the present technology may be prepared, in whole or in part, using a peptide synthesis methods, such as conventional liquid-phase (also known as solution-phase) peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132). The peptidomimetic thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption. For example, the peptidomimetic described herein can be prepared as described in WO2019/118878 entitled: Mitochondrial-Targeting Peptides.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. After coupling, the resin is optionally treated with a capping reagent to thereby cap (render inactive towards subsequent coupling steps) any unreacted amine groups. These steps (i.e., deprotection, coupling and optionally capping) may be repeated to form the desired peptide chain. Once the desired peptide chain is complete, the peptide may be cleaved from the solid support.

In certain embodiments, the protecting groups used on the amino groups of the amino acid residues (of peptides and/or peptidomimetics) include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide and/or peptidomimetic synthesis, but may be removed before, after, or concomitantly with cleavage of the peptide from the solid support (if support bound) or upon final deprotection in the case of solution-phase synthesis. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis (which protection groups can also be used in peptidomimetic synthesis where the peptidomimetic comprises functional groups found in peptides).

In certain embodiments, the solid support material used in the solid-phase peptide synthesis method is a gel-type support such as polystyrene, polyacrylamide, or polyethylene glycol. Alternatively, materials such as controlled-pore glass, cellulose fibers, or polystyrene may be functionalized at their surface to provide a solid support for peptide synthesis.

Coupling reagents that may be used in the solid-phase (or solution-phase) peptide synthesis described herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC) and its HCl salt (EDC·HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include (1-[Bis(dimethylamino)methylene]-1H-1,2,3-triazolo[4,5-b]pyridinium 3-oxide hexafluorophosphate (HATU) and (2-(1H-benzotriazol-1-yl)-1,1,3,3-tetramethyluronium hexafluorophosphate (HBTU), generally used in combination with an organic base such as N,N-diisopropylethylamine (DIEA) and a hindered pyridine-type base such as lutidine or collidine.

In some embodiments, the amino acids can be activated toward coupling to a peptide or peptidomimetic by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996) and WO2018/034901.

In certain exemplary embodiments, compounds useful in the therapeutic methods described herein can be synthesized in a convergent fashion, according to the solid phase synthesis depicted in Scheme 1.

For reference in the following schemes,

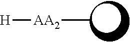

indicates

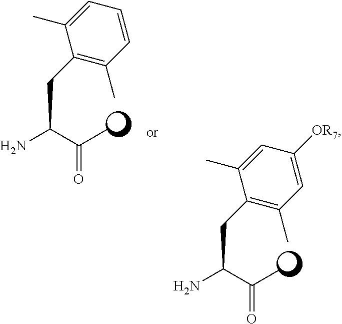

wherein

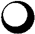

represents a solid support and optionally a linking group.

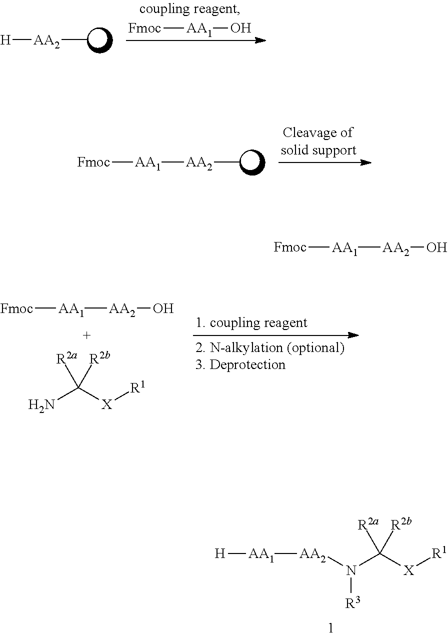

For example, the compound pictured below may be synthesized in such a fashion, as illustrated in Scheme 2.

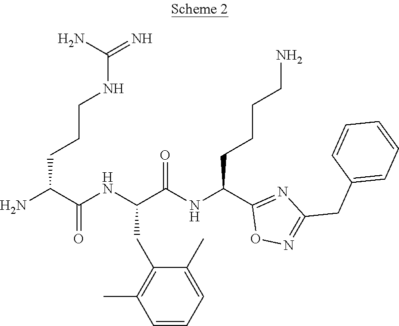 [SMILES: Cc1cccc(C)c1C[C@H](NC(=O)[C@H](N)CCCNC(=N)N)C(=O)N[C@@H](CCCCN)c1nc(Cc2ccccc2)no1]

For reference in the following schemes,

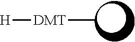

indicates

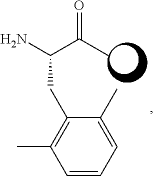

wherein

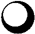

represents a solid support and optionally a linking group.

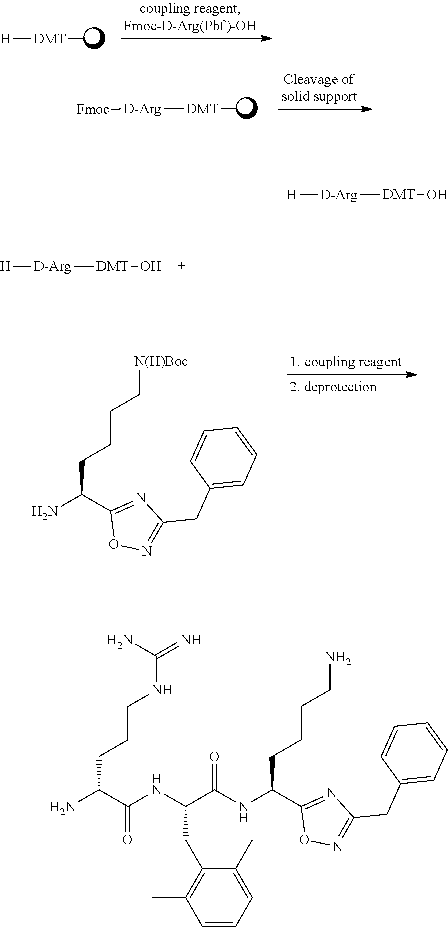

The compounds of the present technology may also be synthesized according to conventional liquid-phase peptide synthetic routes, e.g., according to Scheme 3.

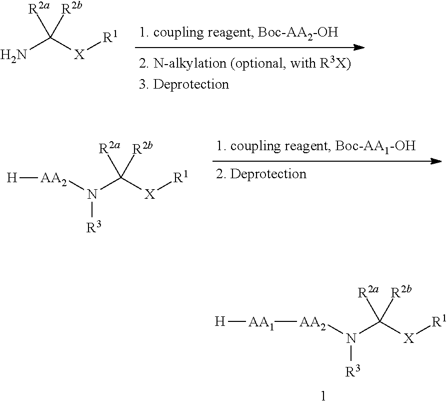

For example, the compound pictured below may be synthesized in such a fashion, as illustrated in Scheme 4.

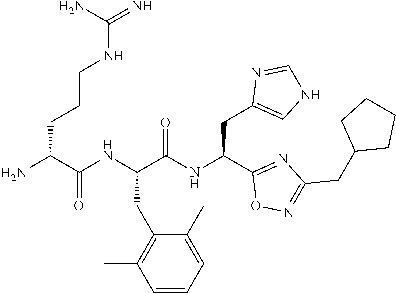 [SMILES: Cc1cccc(C)c1C[C@H](NC(=O)[C@H](N)CCCNC(=N)N)C(=O)N[C@@H](Cc1c[nH]cn1)c1nc(CC2CCCC2)no1]

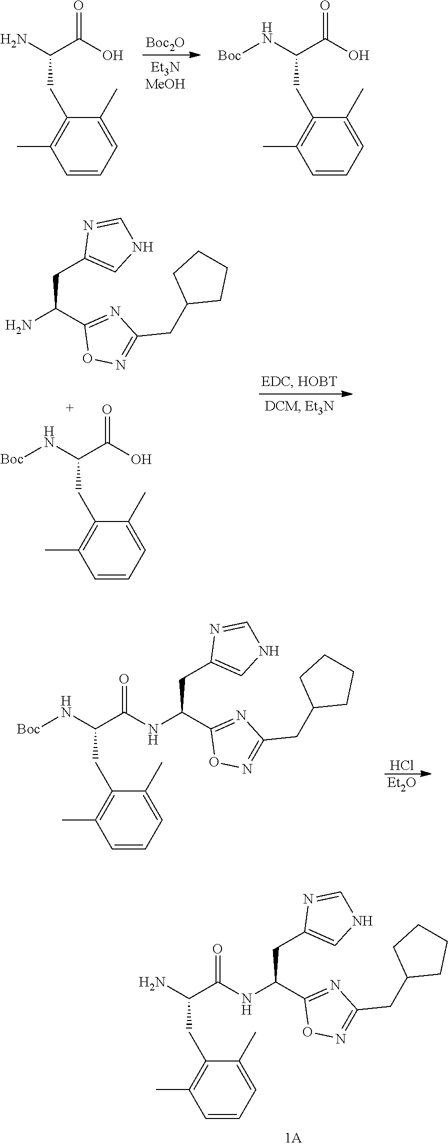

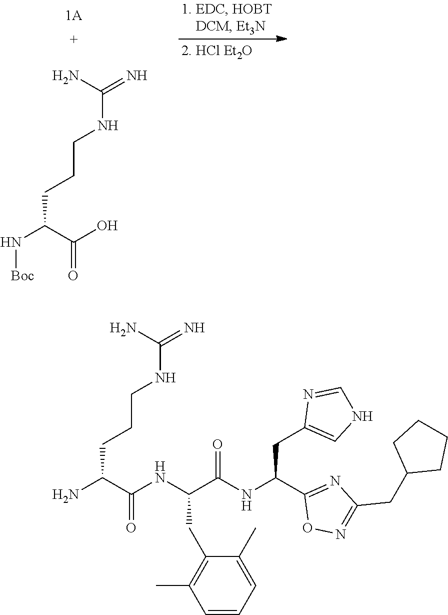 [SMILES: CC(C)(C)OC(=O)N[C@H](CCCNC(=N)N)C(=O)O ; Cc1cccc(C)c1C[C@H](NC(=O)[C@H](N)CCCNC(=N)N)C(=O)N[C@@H](Cc1c[nH]cn1)c1nc(CC2CCCC2)no1]

### Synthesis of (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (D-Arg-DMT-NH((S)-5-amino-1-(3-benzyl-1,2,4-oxadiazol-5-yl)pent-1-yl), 7a (a.k.a. ((Formula IIa))

In some embodiments, Compound 7a (a.k.a. Formula IIa) may be synthesized as illustrated in Scheme 5, below (Also see WO2019/118878, incorporated herein by reference), wherein compound 12a can be prepared as illustrated in Scheme 6, below.

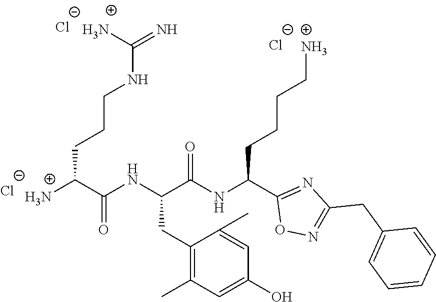 [SMILES: [Cl-] ; [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-]]

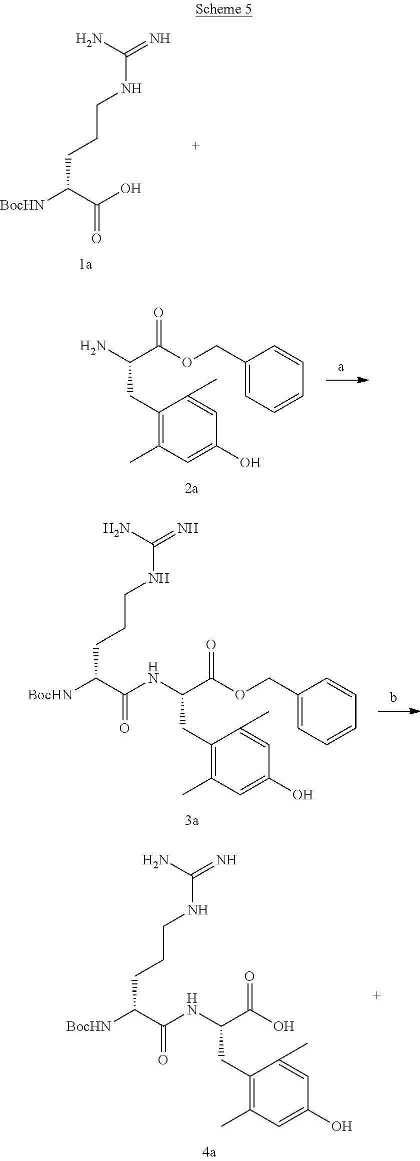 [SMILES: CC(C)(C)OC(=O)N[C@H](CCCNC(=N)N)C(=O)O ; Cc1cc(O)cc(C)c1C[C@H](N)C(=O)OCc1ccccc1 ; Cc1cc(O)cc(C)c1C[C@H](NC(=O)[C@@H](CCCNC(=N)N)NC(=O)OC(C)(C)C)C(=O)OCc1ccccc1 ; Cc1cc(O)cc(C)c1C[C@H](NC(=O)[C@@H](CCCNC(=N)N)NC(=O)OC(C)(C)C)C(=O)O]

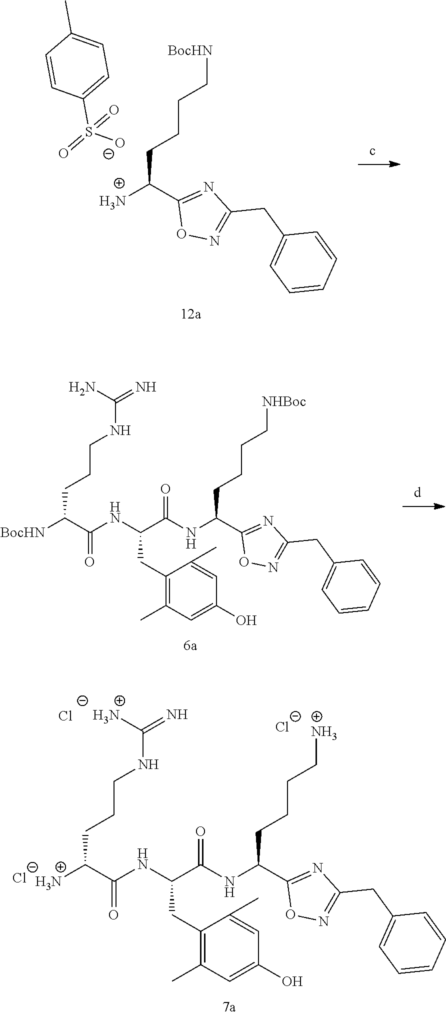

Step a: Synthesis of benzyl (S)-2-((R)-2-((tert-butoxycarbonyl)amino)-5-guanidinopentanamido)-3-(4-hydroxy-2,6-dimethylphenyl)propanoate (3a). To a suspension of 2,6-Dmt-OBn·HCl (2a, 45.0 g, 134 mmol) in ACN (800 mL), NMM (32.7 mL, 298 mmol) was added at 0° C. The reaction mixture was stirred until the reaction mixture became transparent. Then Boc-D-Arg-OH·HCl (1a, 46.3 g, 149 mmol) and HOBt·H₂O (9.11 g, 59.5 mmol) were added to reaction mixture and stirred for 15 min. Finally, EDC·HCl (38.5 g, 201 mmol) was added and mixture was stirred at 0° C. for 4 h. Then EtOAc (450 mL), 1N HCl in brine (300 mL) were added. The combined organic extracts were washed with 1N HCl in brine (7×150 mL), NaHCO₃/brine (300 mL and until pH of aqueous layer is about pH=6 to 7), dried over Na₂SO₄, filtered and concentrated to afford 86.0 g (97%) of Boc-D-Arg-DMT-OBn (3a) that was used without further purification. ¹H-NMR (400 MHz, Methanol-d₄) δ 7.33-7.18 (m, 5H), 6.43 (s, 2H), 5.06 (s, 2H) 4.71 (t, J=7.8 Hz, 1H), 4.07 (t, J=6.7 Hz, 1H), 3.19-3.09 (m, 3H), 3.03-2.97 (m, 1H), 2.23 (s, 6H), 1.72-1.65 (m, 1H), 1.54-1.43 (m, 3H), 1.45 (s, 9H).

Step b: Synthesis of (S)-2-((R)-2-((tert-butoxycarbonyl)amino)-5-guanidinopentanamido)-3-(4-hydroxy-2,6-dimethylphenyl)propanoic acid (4a). To a solution of Boc-D-Arg-DM-Tyr-OBn (3a, 84.0 g, 142 mmol) in MeOH (1000 mL) Pd/C (10% w/w, 14.0 g) was added. The hydrogen was purged in reaction mixture at room temperature for 4 h. Then reaction mixture was filtrated through filter paper and washed with MeOH (150 mL). The solvent was removed by evaporation. White foam product 4a was obtained (74.0 g, 93%) and used without further purification. ¹H-NMR (400 MHz, Methanol-d₄) δ 6.44 (s, 2H), 4.68 (t, J=7.2 Hz, 1H), 4.04 (t, J=6.8 Hz, 1H), 3.15-3.09 (m, 3H), 3.02-2.94 (m, 1H), 2.29 (s, 6H), 1.74-1.59 (m, 1H), 1.54-1.43 (m, 1H), 1.45 (s, 9H).

Step c: Synthesis of tert-butyl ((6R,9S,12S)-1-amino-12-(3-benzyl-1,2,4-oxadiazol-5-yl)-9-(4-hydroxy-2,6-dimethylbenzyl)-1-imino-20,20-dimethyl-7,10,18-trioxo-19-oxa-2,8,11,17-tetraazahenicosan-6-yl)carbamate (6a). DMF (200 mL) was added to 4a (11.17 g, 24 mmol) and stirred at r.t. for 15 min. To the resulting suspension, 12a (10.65 g, 20 mmol) was added and stirred at r.t. for 20 min. After addition of HOBt (612 mg, 4.00 mmol), the suspension was cooled in ice bath. EDC·HCl (5.38 g, 28 mmol) was added in one portion, and the reaction mixture was stirred while cooled in ice bath for 2.5 h and then, for 4.5 h at r.t. The nearly homogeneous reaction mixture was quenched with EtOAc (1500 mL) and the resulting solution was washed for 10 times with brine/aq. 0.5 M HCl (1:1; 400 mL). During the 6th and 9th washings, gel in the aqueous phase was formed. After addition of iPrOH (40 mL in each case) and repeated shaking the layers went clear again. Afterwards, the organic phase was washed for 6 times with brine/sat. aq. NaHCO₃ (9:1; 400 mL). During the 4th washing, gel in the aqueous phase was formed. After addition of iPrOH (40 mL) and repeated shaking the layers were separated easily. The organic phase was washed with brine (200 mL) and water (100 mL) and the solvent was removed under reduced pressure. No vigorous shaking was performed upon washing with water to avoid difficulties in phase separation. As a result, 16.8 g of the crude product were obtained (6a, 97.0% purity by HPLC, white amorphous solid). ¹H-NMR (300 MHz, Methanol-d₄) ppm: 6=7.33-7.16 (m, 5H), 6.38 (s, 2H), 5.18-5.07 (m, 1H), 4.64-4.55 (m, 1H), 4.10-3.92 (m, 3H), 3.18-2.77 (m, 6H), 2.20 (s, 6H), 1.97-1.76 (m, 2H), 1.75-1.14 (m, 8H), 1.43 (s, 9H), 1.41 (s, 9H).

Step d: Synthesis of (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (7a, but also referred to as (Ha the tri-hydrochloride salt of Compound I) herein). After 6a (16.8 g) was dissolved in DCM (100 mL) and cooled to 0° C., TFA (20 mL) was added dropwise and the solution was allowed to stir at 0° C. for 10 min, and then at r.t. for 3 h (LC/MS shows no starting material). Then reaction mixture was evaporated (at 0-5° C.) and additionally re-evaporated from DCM (100 mL, at 0-5° C.). The purification by flash chromatography on reverse phase (cartridge C-18, 120G) was performed on crude material divided in 4 parts. Then all solvents were evaporated at reduced pressure at <40° C. White foam was dissolved in isopropanol (100 mL) and 5 mL of HCl in isopropanol (5-6M) was added at 0° C. and evaporated under reduced pressure. This step was repeated 3 times. Additionally, 100 mL of ACN was added and suspension was evaporated one more time. As a result, white powder of 7a was obtained as the tri-hydrochloride salt. ¹H-NMR (300 MHz, Methanol-d₄) δ 7.36-7.14 (m, 5H), 6.40 (s, 2H), 5.15 (dd, J=8.5, 6.3 Hz, 1H), 4.68 (dd, J=8.7, 7.5 Hz, 1H), 4.07 (s, 2H), 3.97 (t, J=6.3 Hz, 1H), 3.18 (t, J=6.9 Hz, 2H), 3.11 (dd, J=14.2, 8.8 Hz, 1H), 2.95-2.84 (m, 3H), 2.22 (s, 6H), 2.02-1.59 (m, 6H), 1.57-1.28 (m, 4H). MS: EI-MS: m/z 608.4 [M+1].

### Synthesis of (S)-1-(3-Benzyl-1,2,4-oxadiazol-5-yl)-5-((tert-Butoxycarbonyl)amino)pentan-1-Aminium 4-Methylbenzenesulfonate (12a)

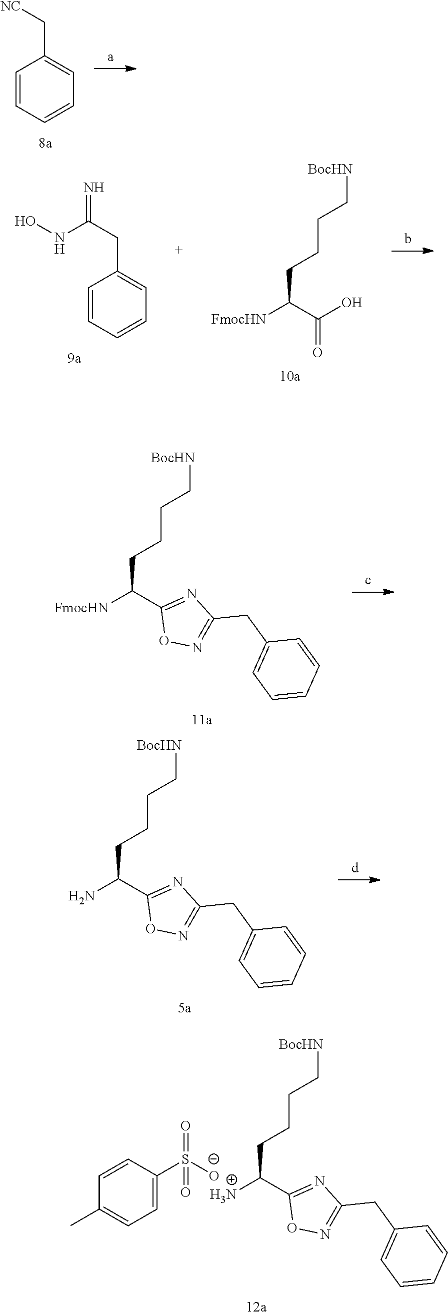

step a: NH₂OH; step b: T₃P, NaHCO₃; step c: TEA; step d: PTSA

Step a: Synthesis of N-hydroxy-2-phenylacetimidamide (9a). To a solution of nitrile 8a (1.0 mol) in EtOH (1.2 L) was added NH₂OH (50% aqueous solution, 130 g, 2.0 mol). The solution was heated to reflux and stirred for 12 hours (hrs.). After completion, the reaction mixture was concentrated under reduced pressure. The resulting residue was re-dissolved in EtOH (350 mL) and concentrated under reduced pressure again (this procedure was repeated three times). The resulting solid was triturated in hexane (350 mL), filtered, washed with hexane (100 mL), and then dried to give the desired product 9a as white solid. (10.5 kg; KF=1295) with good results (purity by HPLC, >98.9 A %; Assay=22.2 w %, yield=91%). ¹H NMR (300 MHz, DMSO-d₆): δ 8.90 (s, 1H), 7.28-7.18 (m, 5H), 5.40 (s, 2H), 3.25 (s, 2H) ppm. MS: (M+H)⁺: m/z=151.1

Step b: Synthesis of (9H-Fluoren-9-yl)methyl tert-Butyl (1-(3-Benzyl-1,2,4-oxadiazol-5-yl)pentane-1,5-diyl) (S)-Dicarbamate (Ha). To a solution of protected enantiomerically pure N2-(((9H-fluoren-9-yl)methoxy)carbonyl)-N6-(tert-butoxycarbonyl)-L-lysine (10a, 4.31 kg, 9.2 mol) and hydroxyimidamide 9a (1.1 equivalents “equiv.” or “eq.”) in ethyl acetate was added NaHCO₃ (3.0 equiv.). The mixture was stirred at 25° C. for 20 minutes (min.). Then, propane phosphonic acid anhydride (T₃P, 50% solution in ethyl acetate, 3.0 equivalents (equiv.)) was added and the reaction mixture was heated to 80° C. and stirred for 4 hrs. (about 60% conversion of compound 10a based on HPLC). Then compound 9a (1.1 equiv.) was added and the reaction mixture was stirred at 80° C. for another 20 hr. (about 10% compound 10a remained). The reaction mixture was cooled to room temperature, saturated aqueous NaHCO₃ (2.0 L) was added, the mixture was then extracted with ethyl acetate (3×1.0 L). The combined organic layers were then washed with brine (1 L), dried over anhydrous Na₂SO₄, filtered and concentrated to give a crude residue, which was generally purified by silica gel column chromatography (Petroleum ether (PE):EtOAc=5:1) to give crude product, (9H-fluoren-9-yl)methyl tert-butyl (1-(3-benzyl-1,2,4-oxadiazol-5-yl)pentane-1,5-diyl) (S)-dicarbamate (11a), solution in ACN (19.7 kg, assay=20%, chiral HPLC purity=99.12 A %, yield=73%). ¹H-NMR (300 MHz, CDCl₃): δ 7.78 (d, J=7.5 Hz, 2H), 7.61 (d, J=6.3 Hz, 2H), 7.42 (t, J=7.5 Hz, 2H), 7.35-7.30 (m, 7H), 5.52 (br, 1H), 5.09-5.05 (m, 1H), 4.56-4.37 (m, 3H), 4.22 (t, J=6.6 Hz, 1H), 4.08 (s, 2H), 1.95-1.86 (m, 2H), 1.48-1.42 (m, 11H) ppm. MS: (M−100+H)⁺: m/z=483.2.

Step c: Synthesis of tert-Butyl (S)-(5-Amino-5-(3-Benzyl-1,2,4-oxadiazol-5-yl)pentyl)-carbamate (5a). To a solution of compound (9H-fluoren-9-yl)methyl tert-butyl (1-(3-benzyl-1,2,4-oxadiazol-5-yl)pentane-1,5-diyl) (S)-dicarbamate (11a) was added TEA (2.5 eq.). The mixture was kept stirring with mechanical stirrer at 20~25° C. for 15 h. The reaction mixture was diluted by tap water and MTBE. Separated, aqueous layer was extracted by MTBE for one time. Both MTBE layers were combined, and then washed by NH₄Cl. Then anhydrous Na₂SO₄ was added and that solution stirred for least 2 h, then filtered and washed with MTBE to afford tert-butyl (S)-(5-amino-5-(3-benzyl-1,2,4-oxadiazol-5-yl)pentyl)-carbamate (5a) solution in MTBE (32.9 kg, assay=6.5%, yield=88%). ¹H-NMR (300 MHz, DMSO-d₆): δ 7.33-7.25 (m, 5H), 6.78 (br, 1H), 5.09-5.05 (m, 1H), 4.56-4.37 (m, 3H), 4.06 (s, 2H), 3.98 (t, J=6.6 Hz, 1H), 2.87-2.84 (m, 2H), 2.10 (s, 2H), 1.38-1.34 (m, 2H), 1.24 (s, 9H), 1.20-1.15 (m, 2H) ppm. MS: (M+H)⁺: m/z=361.1.

Step d: Synthesis of (S)-1-(3-Benzyl-1,2,4-oxadiazol-5-yl)-5-((tert-Butoxycarbonyl)-amino)pentan-1-Aminium 4-Methylbenzenesulfonate (12a). p-toluenesulfonic acid (PTSA) was added to solution of crude tert-butyl (S)-(5-amino-5-(3-benzyl-1,2,4-oxadiazol-5-yl)pentyl)-carbamate (5a) in MTBE to afford (S)-1-(3-benzyl-1,2,4-oxadiazol-5-yl)-5-((tert-butoxycarbonyl)amino)pentan-1-aminium 4-methylbenzenesulfonate (12a) (2.7 kg, yield=85%, HPLC purity>99%, ee>99%) as white solid. ¹H-NMR (400 MHz, DMSO-d₆): δ 8.74 (br, 3H), 7.48 (d, J=8.0 Hz, 2H), 7.37-7.26 (m, 5H), 7.11 (d, J=8.0 Hz, 2H), 6.77 (t, J=5.2 Hz, 1H), 4.82 (t, J=6.8 Hz, 1H), 4.17 (s, 2H), 2.90-2.86 (m, 2H), 2.29 (s, 3H), 1.39-1.36 (m, 11H), 1.35-1.28 (m, 2H) ppm. MS: (M−172+H)⁺: m/z=361.1.

### Therapeutic Methods:

One aspect of the present technology includes methods useful to treat, prevent, inhibit, ameliorate or delay the onset of an ophthalmic disease, disorder or condition in a mammalian subject. Accordingly, in one aspect, the present methods provide for the management of an ophthalmic disease, disorder or condition in a subject by administering an effective amount of a peptidomimetic, such as peptidomimetics of Formula I, or a pharmaceutically acceptable salt, tautomer, hydrate, and/or solvate thereof to a subject in need thereof. For example, a subject can be administered said peptidomimetic (or a composition, formulation or medicament comprising the peptidomimetic) in an effort to improve one or more of the factors or aspects contributing to an ophthalmic disease, disorder or condition, where, for example, the disease, disorder or condition is macular degeneration (including age-related macular degeneration), dry eye, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (e.g., Kearns-Sayre syndrome), and/or Leber congenital amaurosis (LCA). The disease, disorder or condition could also be geometric atrophy (GA). The disease, disorder or condition could also be drusen. The disease, disorder or condition could also be glaucoma.

As discussed above, the ellipsoid zone (EZ) of the eye is mitochondria-rich. The peptidomimetics disclosed herein are mitochondrial-targeted. The peptidomimetics disclosed herein can penetrate into the eye (and its various compartments/parts; e.g. choroid, ciliary body, cornea, fovea, iris, lens, macula, optic nerve, pupil, retina, sclera and vitreous humor) as demonstrated for a compound of Formula II, in Example 1. Thus, in another aspect, as demonstrated by Example 2, below, the peptidomimetics disclosed herein are potentially very beneficial drugs for use in treatment and management of ophthalmic diseases, disorders and conditions, and in particular those affecting the ellipsoid zone. Thus, the present methods also can provide for the management of the deterioration of the ellipsoid zone (i.e., deterioration or ellipsoid zone integrity) in one or more eyes of a mammalian subject.

Thus, one aspect of the technology includes methods of addressing an ophthalmic disease, disorder or condition in a subject for therapeutic purposes. In therapeutic applications, compounds, compositions, formulations or medicaments can be administered to a subject suspected of, or already suffering from such a disease, disorder or condition in an amount sufficient to cure, or at least partially arrest, the symptoms of the disease, including its complications and intermediate pathological phenotypes in development of the disease. As such, the disclosure provides methods for managing an individual afflicted with an ophthalmic disease, disorder or condition.

Thus, in one embodiment, the present technology is directed to a method for treating, preventing, inhibiting, ameliorating or delaying the onset of an ophthalmic disease, disorder or condition in a mammalian subject in need thereof, comprising administering to the subject a therapeutically effective amount of at least one peptidomimetic or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

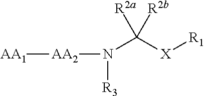

wherein,
AA₁ is selected from

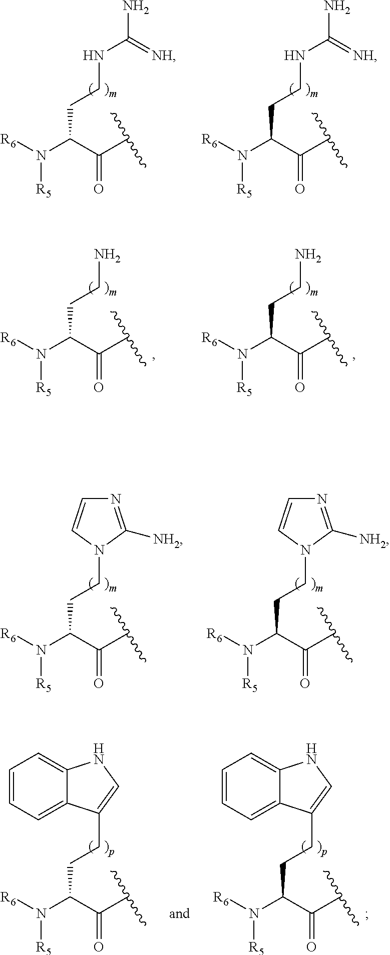

AA₂ is selected from

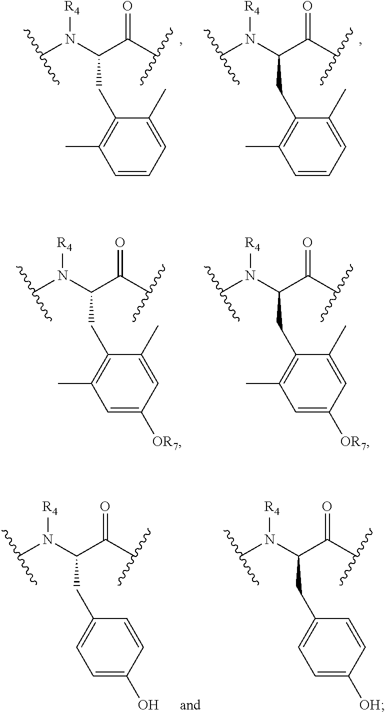

R₁ is selected from

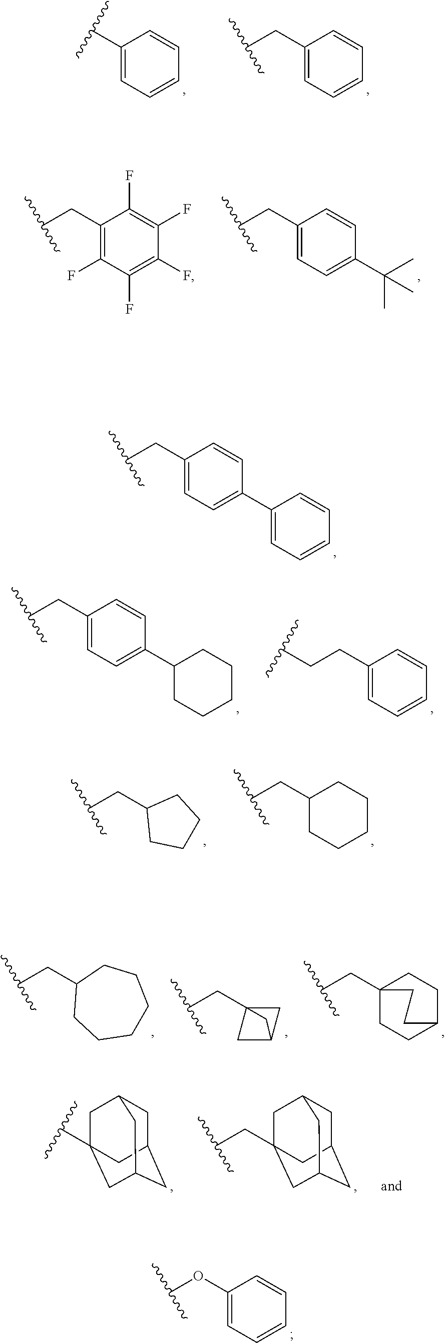

R^{2a} is selected from

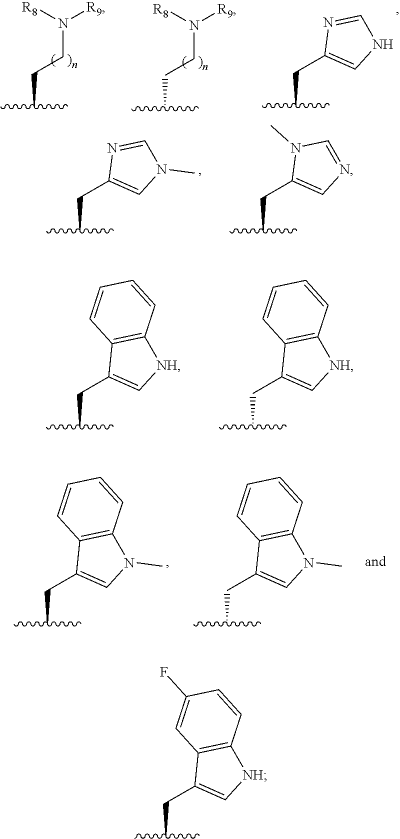

R^{2b} is H or CH₃; R₃ and R₄ are independently selected from H and (C₁-C₆)alkyl; R₅ and R₆ are independently H, methyl, ethyl, propyl, cyclopropyl, or cyclobutyl; or R₅ and R₆ together with the N atom to which they are attached form a 4-6-membered heterocyclyl; R₇ is selected from H, (C₁-C₆)alkyl, cycloalkyl, and aryl; R₈ and R₉ are independently selected from H, (C₁-C₆)alkyl, cycloalkyl, and aryl; or R₈ and R₉ together with the N atom to which they are attached form a 4-6-membered heterocyclyl; m is 1, 2, or 3; n is 1, 2, or 3; p is 0 or 1; X is selected from

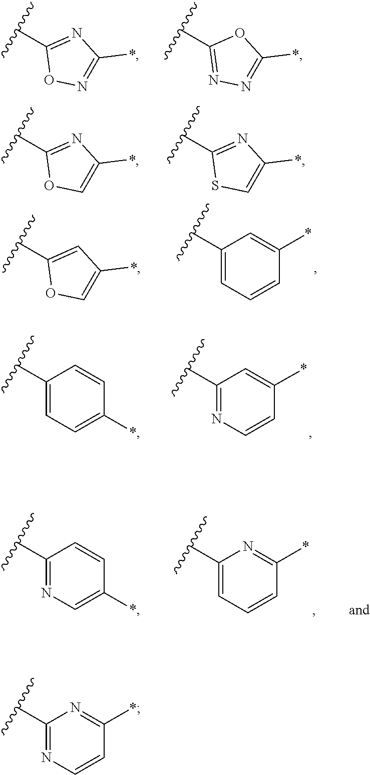

and * denotes the point of attachment of X to R₁, and wherein one or more of the hydrogen atoms of the peptidomimetic is optionally substituted with a deuterium or fluorine atom. In some embodiments, AA₁ is selected from

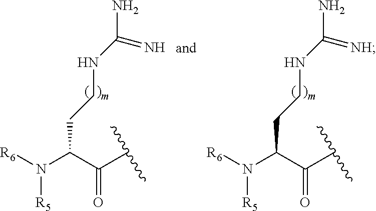

AA₂ is selected from

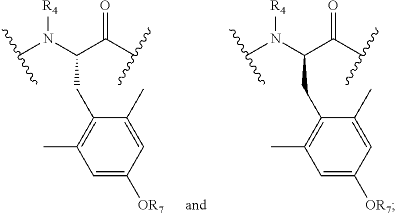

R₁ is selected from

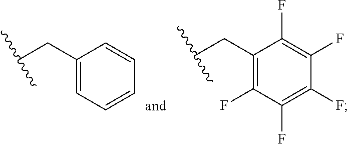 [SMILES: *Cc1ccccc1 ; *Cc1c(F)c(F)c(F)c(F)c1F]

R^{2a} is selected from

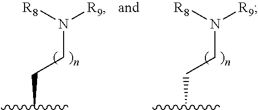

R^{2b} is H; R₃ and R₄ are independently selected from H and methyl; R₅ and R₆ are independently selected from H and methyl; R₇ is selected from H and methyl; R₈ and R₉ are independently selected from H and methyl; and X is selected from

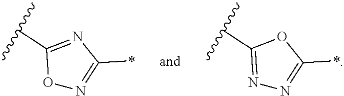

In some embodiments, AA₁ is

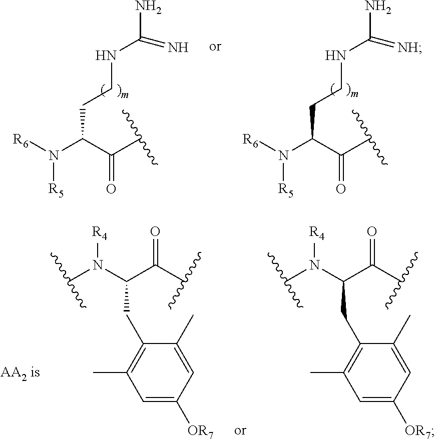

R₁ is

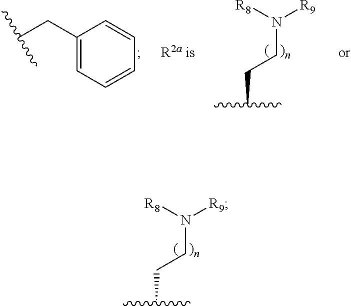

R₇ is H; and X is

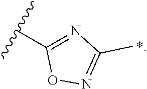 [SMILES: *c1nc(C)no1]

In some embodiments, the peptidomimetic is a peptidomimetic of Formula II, Formula III, Formula IV, Formula V, Formula VI, Formula VII, Formula VIII, Formula IX, Formula X, Formula XI, Formula XII, Formula XIII, Formula XIV or Formula XV;

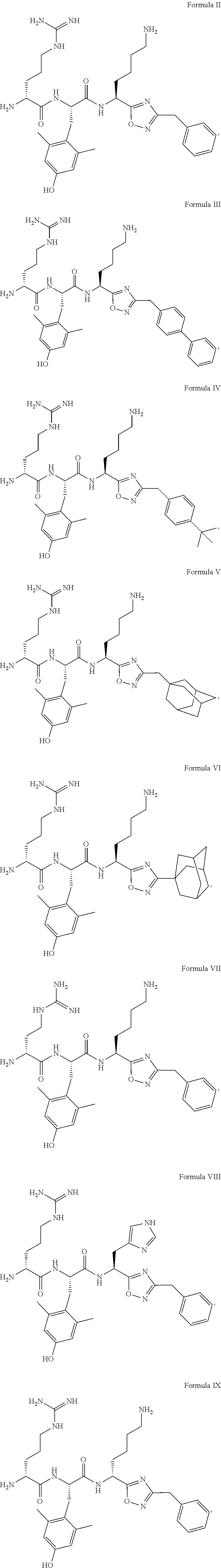

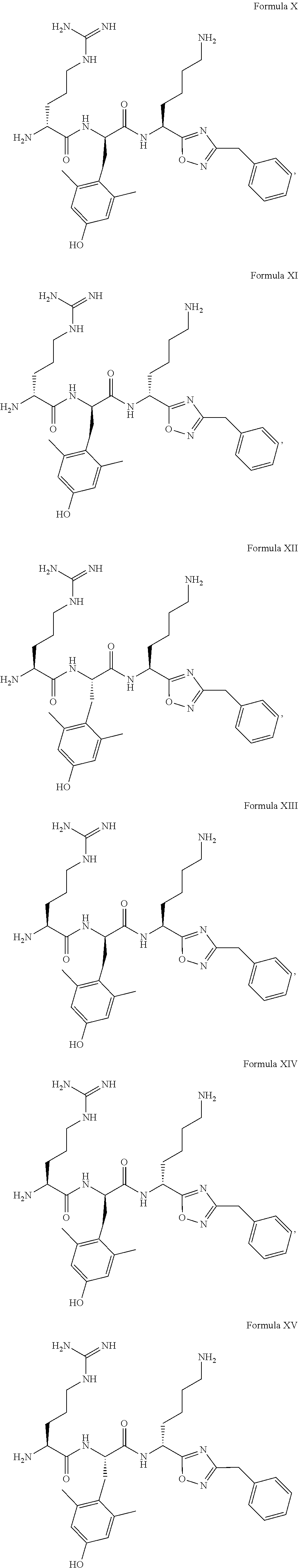

or a pharmaceutically acceptable salt, tautomer, hydrate, and/or solvate thereof and wherein one or more of the hydrogen atoms of the peptidomimetic is optionally substituted with a deuterium or fluorine atom.

In some embodiments, the present technology is directed to a method for treating, preventing, inhibiting, ameliorating or delaying the onset of deterioration of ellipsoid zone integrity in one or more eyes of a mammalian subject in need thereof, comprising administering to the subject a therapeutically effective amount of at least one peptidomimetic, or a pharmaceutically acceptable salt, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

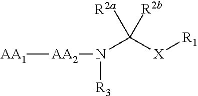

wherein,
AA₁ is selected from

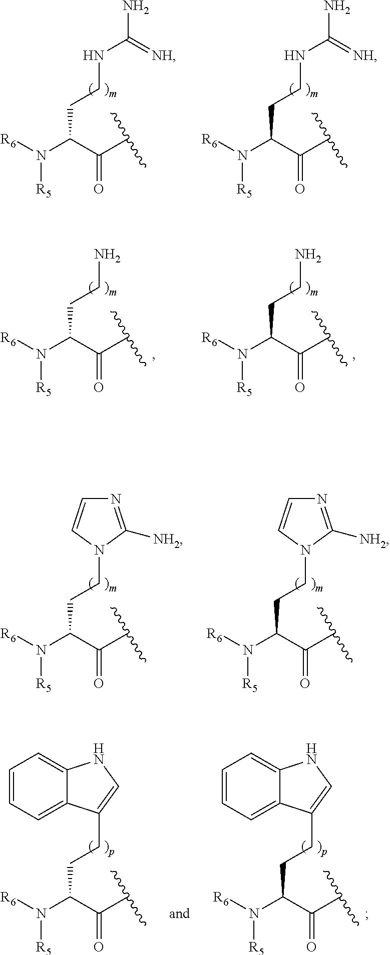

AA₂ is selected from

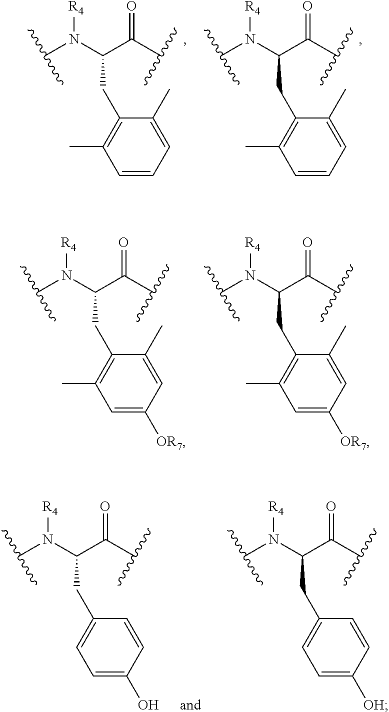

R₁ is selected from

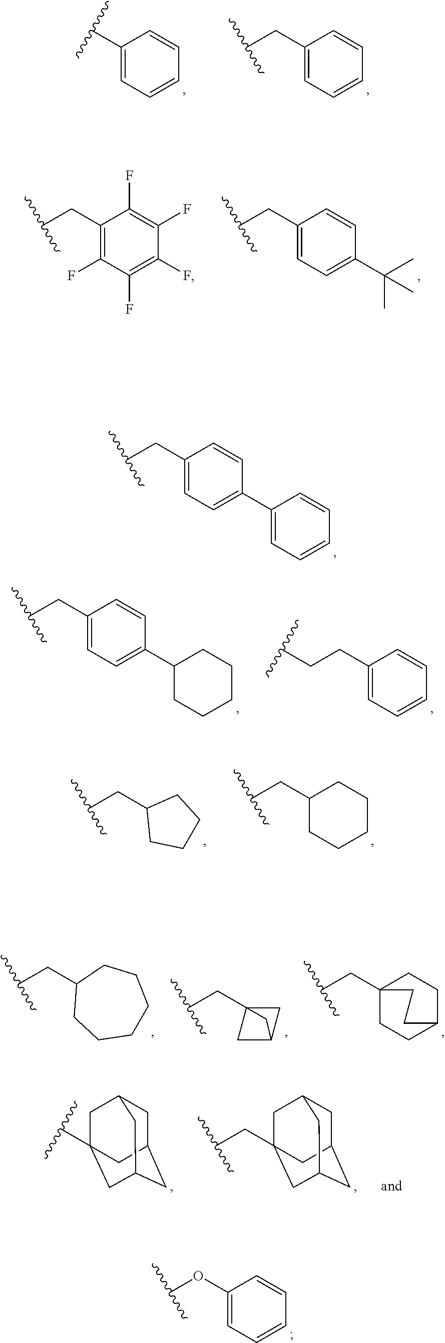

R^{2a} is selected from

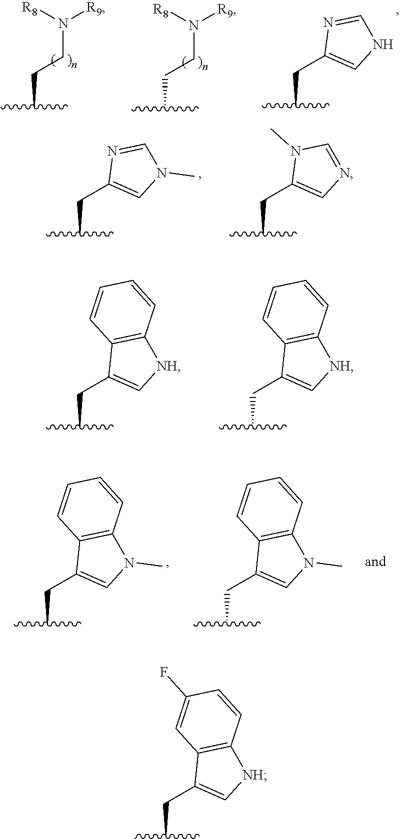

R^{2b} is H or CH₃; R₃ and R₄ are independently selected from H and (C₁-C₆)alkyl; R₅ and R₆ are independently H, methyl, ethyl, propyl, cyclopropyl, or cyclobutyl; or R₅ and R₆ together with the N atom to which they are attached form a 4-6-membered heterocyclyl; R₇ is selected from H, (C₁-C₆)alkyl, cycloalkyl, and aryl; R₈ and R₉ are independently selected from H, (C₁-C₆)alkyl, cycloalkyl, and aryl; or R₈ and R₉ together with the N atom to which they are attached form a 4-6-membered heterocyclyl; m is 1, 2, or 3; n is 1, 2, or 3; p is 0 or 1; X is selected from

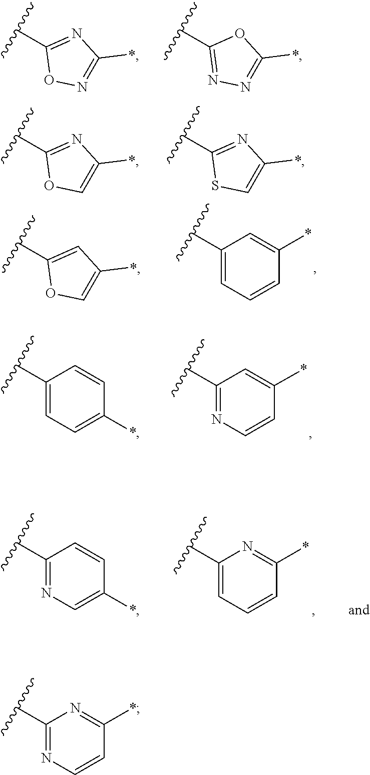

and * denotes the point of attachment of X to R₁, and wherein one or more of the hydrogen atoms of the peptidomimetic is optionally substituted with a deuterium or fluorine atom. In some embodiments, AA₁ is selected from

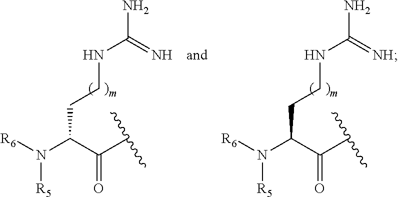

AA₂ is selected from

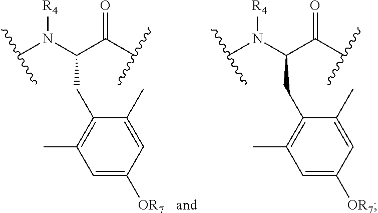

R₁ is selected from

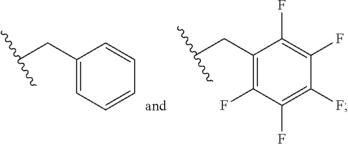 [SMILES: *Cc1ccccc1 ; *Cc1c(F)c(F)c(F)c(F)c1F]

R^{2a} is selected from

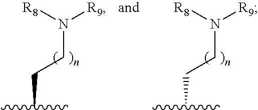

R^{2b} is H; R₃ and R₄ are independently selected from H and methyl; R₅ and R₆ are independently selected from H and methyl; R₇ is selected from H and methyl; R₈ and R₉ are independently selected from H and methyl; and X is selected from

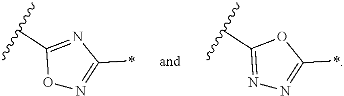

In some embodiments, AA₁ is

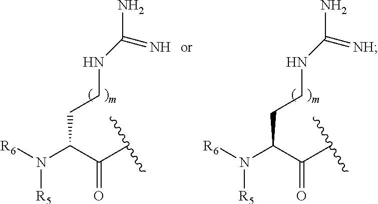

AA₂ is

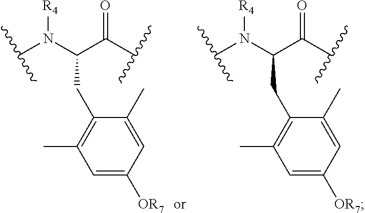

R₁ is

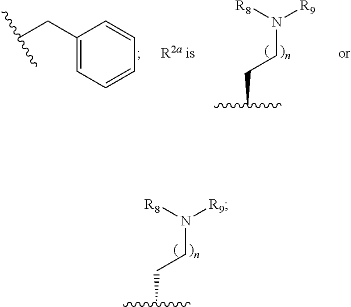

R₇ is H; and X is

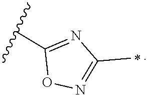 [SMILES: *c1nc(C)no1]

In some embodiments, the peptidomimetic is a peptidomimetic of Formula II, Formula III, Formula IV, Formula V, Formula VI, Formula VII, Formula VIII, Formula IX, Formula X, Formula XI, Formula XII, Formula XIII, Formula XIV or Formula XV;

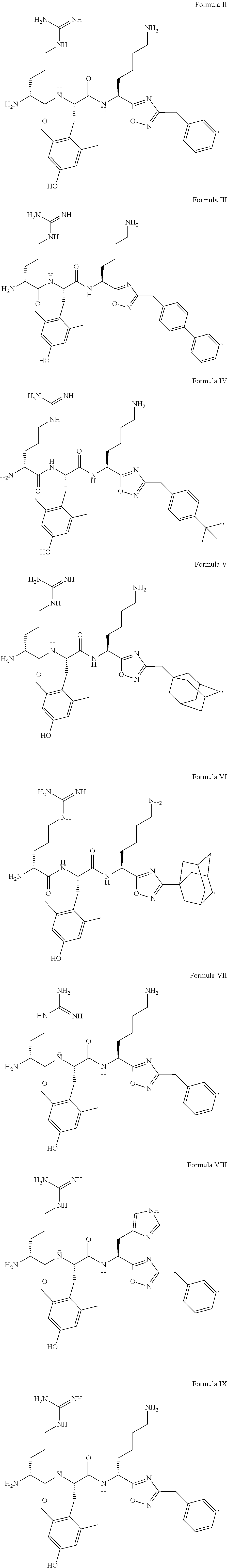

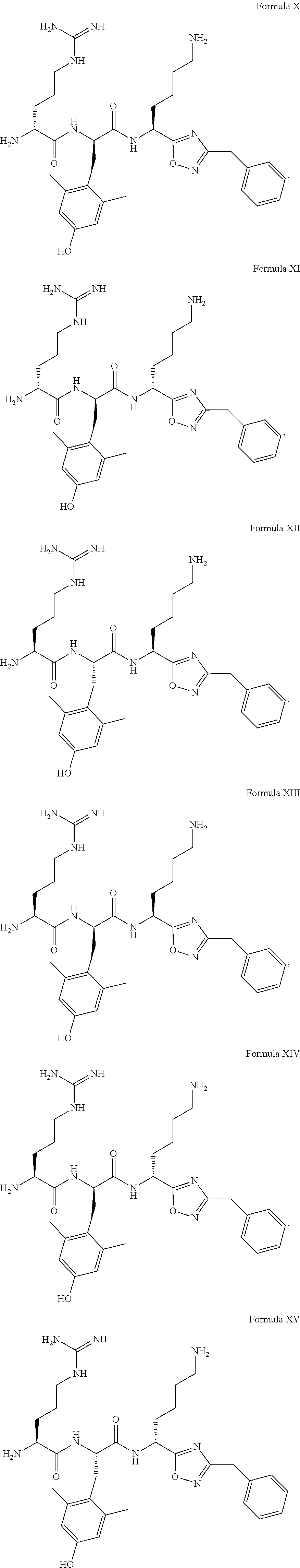

or a pharmaceutically acceptable salt, tautomer, hydrate, and/or solvate thereof and wherein one or more of the hydrogen atoms of the peptidomimetic is optionally substituted with a deuterium or fluorine atom.

In some embodiments, the present technology is directed to a method for treating, preventing, inhibiting, ameliorating or delaying the onset of geometric atrophy a mammalian subject in need thereof where the subject has been diagnosed with age-related macular degeneration (AMD), comprising administering to the subject a therapeutically effective amount of at least one peptidomimetic, or a pharmaceutically acceptable salt, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

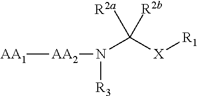

wherein,
AA₁ is selected from

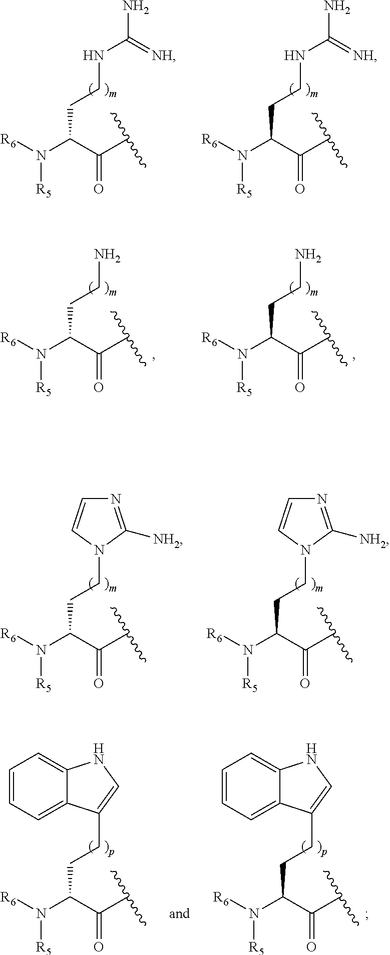

AA₂ is selected from

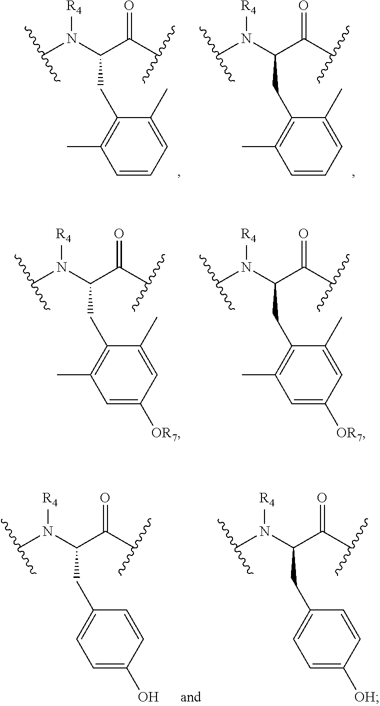

R₁ is selected from

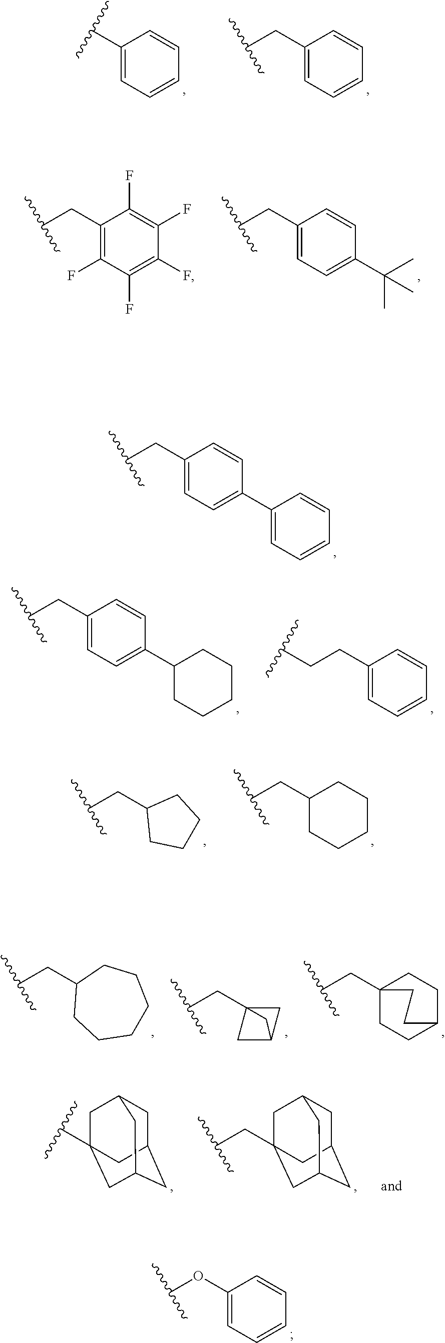

R^{2a} is selected from

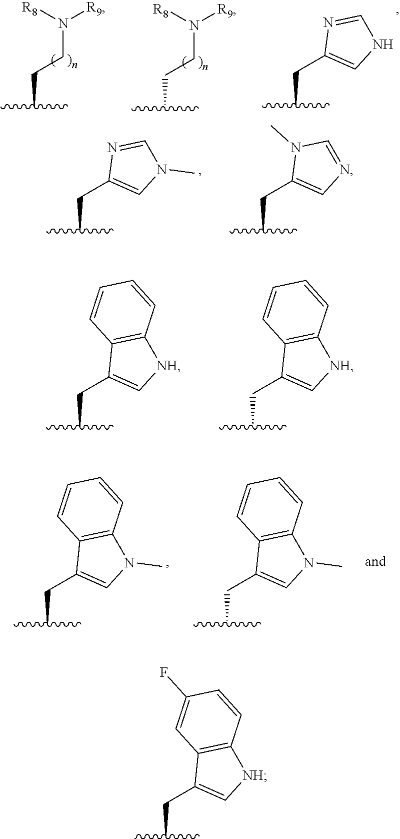

R^{2b} is H or CH₃; R₃ and R₄ are independently selected from H and (C₁-C₆)alkyl; R₅ and R₆ are independently H, methyl, ethyl, propyl, cyclopropyl, or cyclobutyl; or R₅ and R₆ together with the N atom to which they are attached form a 4-6-membered heterocyclyl; R₇ is selected from H, (C₁-C₆)alkyl, cycloalkyl, and aryl; R₈ and R₉ are independently selected from H, (C₁-C₆)alkyl, cycloalkyl, and aryl; or R₈ and R₉ together with the N atom to which they are attached form a 4-6-membered heterocyclyl; m is 1, 2, or 3; n is 1, 2, or 3; p is 0 or 1; X is selected from

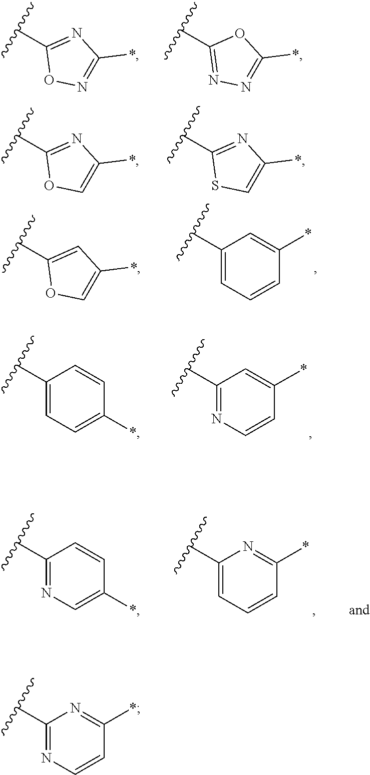

and * denotes the point of attachment of X to R₁, and wherein one or more of the hydrogen atoms of the peptidomimetic is optionally substituted with a deuterium or fluorine atom. In some embodiments, AA₁ is selected from

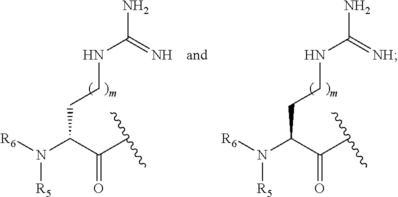

AA₂ is selected from

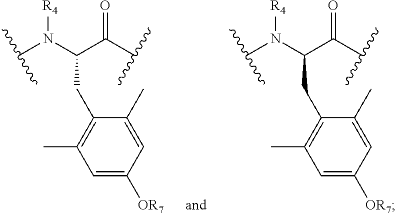

R₁ is selected from

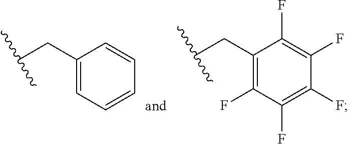 [SMILES: *Cc1ccccc1 ; *Cc1c(F)c(F)c(F)c(F)c1F]

R^{2a} is selected from

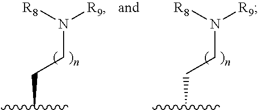

R^{2b} is H; R₃ and R₄ are independently selected from H and methyl; R₅ and R₆ are independently selected from H and methyl; R₇ is selected from H and methyl; R₈ and R₉ are independently selected from H and methyl; and X is selected from

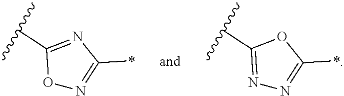

In some embodiments, AA₁ is

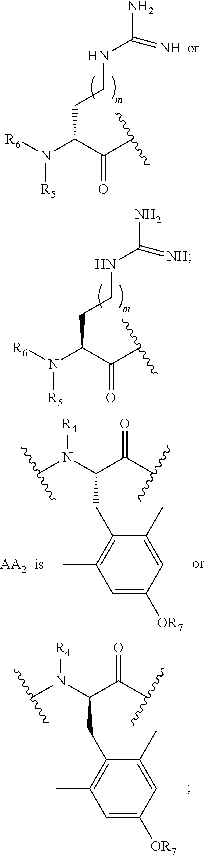

R₁ is

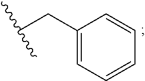 [SMILES: *Cc1ccccc1]

R^{2a} is

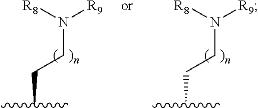

R₇ is H; and X is

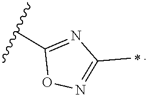 [SMILES: *c1nc(C)no1]

In some embodiments, the peptidomimetic is a peptidomimetic of Formula II, Formula III, Formula IV, Formula V, Formula VI, Formula VII, Formula VIII, Formula IX, Formula X, Formula XI, Formula XII, Formula XIII, Formula XIV or Formula XV;

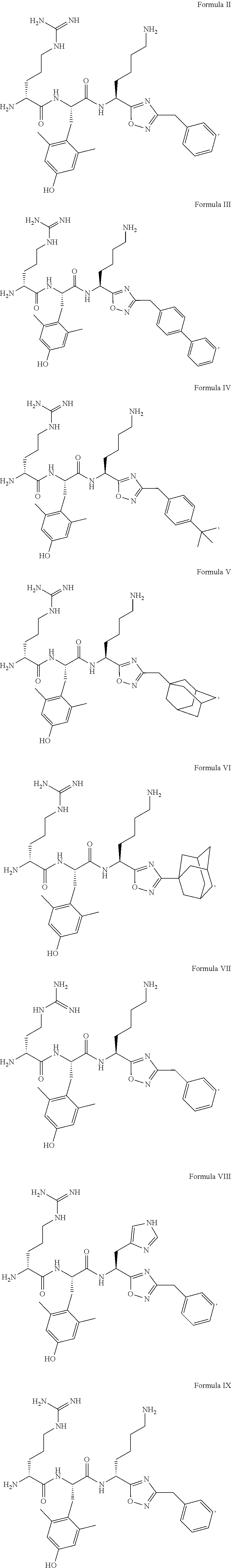

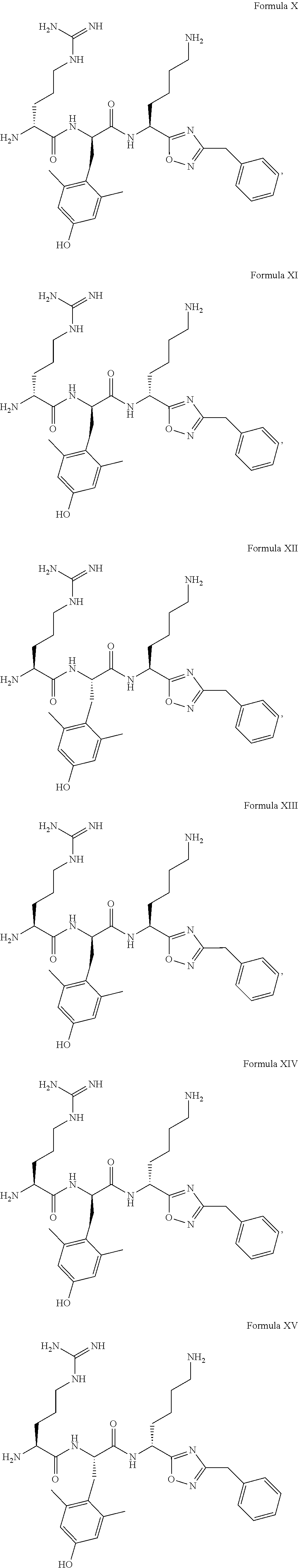

or a pharmaceutically acceptable salt, tautomer, hydrate, and/or solvate thereof and wherein one or more of the hydrogen atoms of the peptidomimetic is optionally substituted with a deuterium or fluorine atom.

In some embodiments of any of the foregoing methods, a peptidomimetic is administered to a subject having, or suspected of having, macular degeneration (including, but not limited to, age-related macular degeneration). Macular degeneration is typically an age-related disease. The general categories of macular degeneration include wet, dry, and non-aged related macular degeneration. Dry macular degeneration, which accounts for about 80-90 percent of all cases, is also known as atrophic, nonexudative, or drusenoid macular degeneration. With dry macular degeneration, drusen typically accumulate beneath the retinal pigment epithelium tissue. Vision loss subsequently occurs when drusen interfere with the function of photoreceptors in the macula. Symptoms of dry macular generation include, but are not limited to, distorted vision, center-vision distortion, light or dark distortion, and/or changes in color perception. Dry macular degeneration can result in the gradual loss of vision. Specific damage to the retinal pigmented epithelial (RPE) cells is a hallmark of age-related macular degeneration (AMD), and RPE cell cultures are frequently used as in vitro models of dry AMD.

Wet macular degeneration is also known as neovascularization, subretinal neovascularization, exudative, or disciform degeneration. With wet macular degeneration, abnormal blood vessels grow beneath the macula. The blood vessels leak fluid into the macula and damage photoreceptor cells. Wet macular degeneration can progress rapidly and cause severe damage to central vision. Wet and dry macular degeneration have identical symptoms. Non-age related macular degeneration, however, is rare and may be linked to heredity, diabetes, nutritional deficits, injury, infection, or other factors. The symptoms of non-age related macular degeneration also include, but are not limited to, distorted vision, center-vision distortion, light or dark distortion, and/or changes in color perception.

In some embodiments of any of the foregoing methods, a peptidomimetic is administered to a subject having, or suspected of having, dry eye. Approximately 20 million Americans suffer from Dry Eye Disease. People with Dry Eye Disease produce poor quality tears and/or do not produce a sufficient quantity of tears to supply nourishment and to lubricate the cornea, leading to eyes that look red and feel chronically irritated, gritty, and scratchy. Dry Eye Disease is not just insufficient and/or poor quality tears. The condition is associated with inflammation and tissue damage, believed to be caused, at least in part, by oxidative stress.

In some embodiments of any of the foregoing methods, a peptidomimetic is administered to a subject having, or suspected of having, diabetic retinopathy. Diabetic retinopathy is characterized by capillary microaneurysms and dot hemorrhaging. Thereafter, microvascular obstructions cause cotton wool patches to form on the retina. Moreover, retinal edema and/or hard exudates may form in individuals with diabetic retinopathy due to increased vascular hyperpermeability. Subsequently, neovascularization appears and retinal detachment is caused by traction of the connective tissue grown in the vitreous body. Iris rubeosis and neovascular glaucoma may also occur which, in turn, can lead to blindness. The symptoms of diabetic retinopathy include, but are not limited to, difficulty reading, blurred vision, sudden loss of vision in one eye, seeing rings around lights, seeing dark spots, and/or seeing flashing lights.

In some embodiments of any of the foregoing methods, a peptidomimetic is administered to a subject having, or suspected of having, diabetic macular edema. Diabetic macular edema involves damage to the blood vessels in the retina that progress to a point where they leak fluid into the macula thereby causing the macula to swell and this results in blurred vision.

In some embodiments of any of the foregoing methods, a peptidomimetic is administered to a subject having, or suspected of having, cataracts. Cataracts is a congenital or acquired disease characterized by a reduction in natural lens clarity. Individuals with cataracts may exhibit one or more symptoms, including, but not limited to, cloudiness on the surface of the lens, cloudiness on the inside of the lens, and/or swelling of the lens. Typical examples of congenital cataract-associated diseases are pseudo-cataracts, membrane cataracts, coronary cataracts, lamellar cataracts, punctuate cataracts, and filamentary cataracts. Typical examples of acquired cataract-associated diseases are geriatric cataracts, secondary cataracts, browning cataracts, complicated cataracts, diabetic cataracts, and traumatic cataracts. Acquired cataracts is also inducible by electric shock, radiation, ultrasound, drugs, systemic diseases, and nutritional disorders. Acquired cataracts further includes postoperative cataracts.

In some embodiments of any of the foregoing methods, a peptidomimetic is administered to a subject having, or suspected of having, autosomal dominant optic atrophy (DOA). DOA is a genetic X-linked neuro-ophthalmic condition characterized by bilateral degeneration of optic nerves. It affects approximately 1 in 10,000 (Denmark) to 1 in 30,000 (worldwide) persons. The nerve damage causes visual loss. It generally begins to manifest itself during the first decade of life and progresses thereafter. The disease itself affects primarily the retinal ganglion nerves. Mutations in the genes known as OPA1 and OPA3, which encode inner mitochondrial membrane proteins (resulting in mitochondrial dysfunction), are generally associated with DOA

In some embodiments of any of the foregoing methods, a peptidomimetic is administered to a subject having, or suspected of having, Leber Hereditary Optic Neuropathy (LHON). LHON is a genetically-based inherited disease that generally starts to manifest itself between the ages of 15 and 35. In LHON, mitochondrial mutations affect complex I subunit genes in the respiratory chain leading to selective degeneration of retinal ganglion cells (RGCs) and optic atrophy generally within a year of disease onset. LHON is caused by mutations in the MT-NDI1, MT-ND4, MT-ND4L and MT-ND6 genes; all of which are associated with mitochondrial genome coding. LHOH affects approximately 1 in 50,000 people worldwide. It generally starts in one eye and progresses quickly to the other eye. Subjects with LHON may eventually become legally or totally blind, often before they turn 50. LHON affects vision needed for tasks such as reading, driving and recognizing others.

In some embodiments of any of the foregoing methods, a peptidomimetic is administered to a subject having, or suspected of having, pigmentary retinopathy (PR). PR is a frequent feature of retinitis pigmentosa. Pigmentary retinopathy is a non-specific finding that may be found in several mitochondrial diseases, such as Neurogenic weakness, Ataxia, and Retinitis Pigmentosa (NARP). PR is an inherited degenerative disorder of the retina, characterized by progressive photoreceptor damage. The damage leads to atrophy and cell death of the photoreceptors. Patients with PR can follow an autosomal-dominate, autosomal recessive or X-linked recessive pattern. The prevalence is about one in about three to four thousand individuals. Symptoms of the disease include nyctalopia (night blindness), peripheral visual field constriction, and sometimes loss of the central visual acuity or visual field

In some embodiments of any of the foregoing methods, a peptidomimetic is administered to a subject having, or suspected of having, retinitis pigmentosa. Retinitis pigmentosa is a disorder that is characterized by rod and/or cone cell damage. The presence of dark lines in the retina is typical in individuals suffering from retinitis pigmentosa. Individuals with retinitis pigmentosa also present with a variety of symptoms including, but not limited to, headaches, numbness or tingling in the extremities, light flashes, and/or visual changes. See, e.g., Heckenlively et al., Clinical findings and common symptoms in retinitis pigmentosa. Am J Ophthalmol. 105(5): 504-511 (1988).

In some embodiments of any of the foregoing methods, a peptidomimetic is administered to a subject having, or suspected of having, glaucoma. Glaucoma is a disease characterized by an increase in intraocular pressure, which leads to a decrease in vision. Elevated pressure affects not only the optic nerve but also the retinal ganglion cells (RGCs) of the retina. Some possible in vitro systems that can be used to evaluate treatments for glaucoma are in-vitro RGC-based. Glaucoma may emanate from various ophthalmologic conditions that are already present in an individual, such as, wounds, surgery, and other structural malformations. Although glaucoma can occur at any age, it frequently develops in elderly individuals and leads to blindness. Glaucoma patients typically have an intraocular pressure in excess of 21 mmHg. However, normal tension glaucoma, where glaucomatous alterations are found in the visual field and optic papilla, can occur in the absence of such increased intraocular pressures, i.e., greater than 21 mmHg. Symptoms of glaucoma include, but are not limited to, blurred vision, severe eye pain, headache, seeing haloes around lights, nausea, and/or vomiting.

In some embodiments of any of the foregoing methods, a peptidomimetic is administered to a subject having, or suspected of having, ocular hypertension. An intraocular pressure (IOP) of over 21 mmHg without optic nerve damage is known as ocular hypertension. Elevated IOP due to inadequate ocular drainage is the primary cause of glaucoma.

In some embodiments of any of the foregoing methods, a peptidomimetic is administered to a subject having, or suspected of having, Uveitis. Uveitis is array of intraocular inflammatory diseases of the eye that often results in irreversible visual loss. Uveitis is responsible for an estimated 30,000 new cases of legal blindness annually in the USA. It is believed that this disease is at least in part due to retinal tissue damage caused excessive mitochondrial oxidative stress that triggers a damaging immune response.

In some embodiments of any of the foregoing methods, a peptidomimetic is administered to a subject having, or suspected of having, choroidal neovascularization. Choroidal neovascularization (CNV) is a disease characterized by the development of new blood vessels in the choroid layer of the eye. The newly formed blood vessels grow in the choroid, through the Bruch membrane, and invade the subretinal space. CNV can lead to the impairment of sight or complete loss of vision. Symptoms of CNV include, but are not limited to, seeing flickering, blinking lights, or gray spots in the affected eye or eyes, blurred vision, distorted vision, and/or loss of vision.

In some embodiments of any of the foregoing methods, a peptidomimetic is administered to a subject having, or suspected of having, retinal degeneration. Retinal degeneration is a disease that relates to the break-down of the retina. Retinal tissue may degenerate for various reasons, such as, artery or vein occlusion, diabetic retinopathy, retinopathy of prematurity, and/or retrolental fibroplasia. Retinal degradation generally includes retinoschisis, lattice degeneration, and is related to progressive macular degeneration. The symptoms of retina degradation include, but are not limited to, impaired vision, loss of vision, night blindness, tunnel vision, loss of peripheral vision, retinal detachment, and/or light sensitivity.

In some embodiments of any of the foregoing methods, a peptidomimetic is administered to a subject having, or suspected of having, Stargardt's disease. also known as Stargardt macular dystrophy, juvenile macular degeneration, or fundus flavimaculatus, is a rare genetic disorder affecting 1 in 8-10 thousand people, that causes progressive degeneration of the macula. Stargardt's disease typically causes vision loss during childhood or adolescence, although in some forms, vision loss may not be noticed until later in adulthood. Mutations in the ABCA4 gene are the most common cause of Stargardt's disease. This gene makes a protein that normally clears away vitamin A byproducts inside photoreceptors. Cells that lack the ABCA4 protein accumulate clumps of lipofuscin, a fatty substance that forms yellowish flecks. As the clumps of lipofuscin increase in and around the macula, central vision becomes impaired. Eventually, these fatty deposits lead to the death of photoreceptors and vision becomes further impaired. Other forms of Stargardt's disease are associated with mutations in the ELOVL4 gene or the PROM1 gene. Fundus flavimaculatus (FFM) is an allelic subtype of Stargardt disease that has been associated with mutation in the ABCA4 gene and the PRPH2 gene. Stargardt's disease is one of the most frequent causes of macular degeneration in childhood. It has onset between 7 and 12 years, a rapidly progressive course, and a poor final visual outcome. Although visual acuity is severely reduced, peripheral visual fields remain normal throughout life. Fundus flavimaculatus, which is a form of fleck fundus disease, derives its name from the occurrence of many yellow spots rather uniformly distributed over the fundus. In some older patients the flecks fade with time as atrophy of the retinal pigment epithelium (RPE) increases. Round, linear, or pisciform lesions are distributed in the posterior pole, sometimes with extension to the equator, and with macular involvement. Network atrophy of the retinal pigment epithelium, and choroidal vascular atrophy are features. Central visual loss, loss of color vision, photophobia, paracentral scotoma, and slow dark adaptation are features.

In some embodiments of any of the foregoing methods, a peptidomimetic is administered to a subject having, or suspected of having, Kearns-Sayre syndrome. Kearns-Sayre syndrome is a condition that affects many parts of the body, especially the eyes. The features of Kearns-Sayre syndrome usually appear before age 20, and the condition is diagnosed by a few characteristic signs and symptoms. People with Kearns-Sayre syndrome have progressive external ophthalmoplegia. Affected individuals also have an eye condition called pigmentary retinopathy, which results from breakdown (degeneration) of the retina that gives it a speckled and streaked appearance.

In some embodiments of any of the foregoing methods, a peptidomimetic is administered to a subject having, or suspected of having, Leber congenital amaurosis (LCA). LCA comprises a group of early-onset childhood retinal dystrophies characterized by vision loss, nystagmus, and severe retinal dysfunction. LCA is a progressive autosomal recessive disease marked by loss of photoreceptors, declining visual fields, and flat electroretinography (ERG) tracings. Most patients are profoundly blind by the second decade of life. Patients usually present at birth with profound vision loss and pendular nystagmus. Electroretinogram (ERG) responses are usually nonrecordable. Other clinical findings may include high hypermetropia, photodysphoria, oculodigital sign, keratoconus, cataracts, and a variable appearance to the fundus. Different subtypes of LCA have been described. The different subtypes are caused by mutations in different genes. Some of these subtypes are also distinguished by their patterns of vision loss and related eye abnormalities. Treatment includes correction farsightedness and use of low-vision aids when possible. In some forms of LCA, the underlying defect is in the RPE65 gene, encoding an isomerohydrolase that is expressed in RPE cells and responsible for generation of 11-cis retinal. Without a functioning RPE65, the RPE cell cannot deliver Vitamin A to the photoreceptors.

### Prophylactic Methods:

Eye disease is generally progressive, often leading to loss of vision that is so complete it becomes impossible to recognize objects and people. In extreme cases, total blindness can result. Sometimes the disease, disorder or condition progresses slowly and sometimes more quickly. Administration of a drug that slows the progression (i.e. prevents progression, inhibits progression, ameliorates progression or delays the onset of a certain condition associated with progression of the disease or disorder) of any loss of vision would be very beneficial to a subject having an ophthalmic disease, disorder or condition that results in progressive vision loss.

Thus, administration or the peptidomimetic according to the methods disclosed above can be considered prophylactic in the sense that they delay progression of the loss of eyesight of the subject. Thus, in one aspect, the present technology provides a methods for preventing, inhibiting, ameliorating or delaying the onset of an ophthalmic disease, disorder or condition in a subject that leads to progressive loss of vision by administering to the subject a peptidomimetic, or a pharmaceutically acceptable salt, tautomer, hydrate, and/or solvate thereof.

Subjects at risk for an ophthalmic disease, disorders or conditions can be identified by, e.g., any or a combination of diagnostic or prognostic assays. In prophylactic applications, pharmaceutical compounds, compositions or medicaments comprising a peptidomimetic, such as a peptidomimetic of Formula I, or a pharmaceutically acceptable salt, stereoisomer, tautomer, hydrate, and/or solvate thereof are administered to a subject susceptible to, or otherwise at risk of a disease, disorder or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Administration of a peptidomimetic prophylactically can occur prior to the manifestation of symptoms characteristic of the aberrancy, such that a disease or disorder is prevented or, alternatively, inhibited, ameliorated, or delayed in its progression. Depending upon the type of aberrancy, a peptidomimetic, such as a peptidomimetic of Formula I, or a pharmaceutically acceptable salt, stereoisomer, tautomer, hydrate, and/or solvate thereof, which acts to enhance or improve mitochondrial function or reduce oxidative damage can be used for treating the subject. The appropriate compound can be determined based on screening assays disclosed in the art.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with macular degeneration (including, without limitation, (wet or dry) age-related macular degeneration).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with dry eye.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with diabetic retinopathy.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with diabetic macular edema.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with cataracts.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with autosomal dominant optic atrophy (DOA).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Leber hereditary optic neuropathy (LHON).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Leber hereditary optic neuropathy (LHON).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Leber hereditary optic neuropathy (LHON).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with pigmentary retinopathy.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with retinitis pigmentosa.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with glaucoma.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with ocular hypertension.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with uveitis.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with chronic progressive external ophthalmoplegia.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Kearns-Sayre syndrome.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Leber congenital amaurosis (LCA).

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with choroidal neovascularization.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with retinal degeneration.

In some embodiments, a peptidomimetic (or a formulation or medicament comprising a peptidomimetic) of Formula I, Formula (II), Formula III, Formula IV, Formula V, Formula VI, Formula VII, Formula VIII, Formula IX, Formula X, Formula XI, Formula XII, Formula XIII, Formula XIV or Formula V, or a pharmaceutically acceptable salt, tautomer, hydrate, and/or solvate thereof is administered to a subject to prevent, inhibit, ameliorate, or delay the onset of vision loss associated with Stargardt's disease.

### Uses, Compositions, Formulations & Medicaments:

The peptidomimetics disclosed herein can be administered in a formulation or medicament (which are also referred to herein as compositions). Alternatively, a composition generally refers to a mixture that contains the peptidomimetic but also contains other compounds such as solvents or the components intended to aid in preparing a formulation or medicament. The formulations or medicaments can be used in any of the methods described above. Typically the formulation or medicament is prepared specifically for use in the management of the particular disease, disorder or condition to be addressed.

In some embodiments, the composition, formulation or medicament is produced by dissolving or suspending the peptidomimetic in a diluent, adjuvant, excipient, or vehicle, such as water or a solvent mixture comprising water. In some embodiments, the formulation or medicament further comprises a preservative. In some embodiments, the preservative is present in the formulation or medicament in a concentration of less than 1% (wt./vol.). In some embodiments, the peptidomimetic(s) is/are present in the formulation or medicament at a concentration of less than 1% (wt./vol.). In some embodiments, the peptidomimetic(s) is/are present in the formulation or medicament in a concentration of between 0.5 and 1% (wt./vol.), inclusive. In some embodiments, the peptidomimetic(s) is/are is present in the formulation or medicament in a concentration of between 1 and 2% (wt./vol.), inclusive. In some embodiments, the peptidomimetic(s) is/are present in the formulation or medicament in a concentration of between 2 and 3% (wt./vol.), inclusive. In some embodiments, the peptidomimetic(s) is/are present in the formulation or medicament in a concentration of between 3 and 5% (wt./vol.), inclusive. In some embodiments, the peptidomimetic(s) is/are present in the formulation or medicament in a concentration above 5% (wt./vol.). In some embodiments, the peptidomimetic(s) is/are present in the formulation or medicament in a concentration above 10% (wt./vol.).

Thus, in one aspect, the present disclosure provides for use of a composition in the preparation of a formulation or medicament for treating, preventing, inhibiting, ameliorating or delaying the onset of: (i) an ophthalmic disease, disorder or condition; or (ii) deterioration of ellipsoid zone integrity in one or more eyes in a mammalian subject in need thereof, wherein the composition comprises a therapeutically effective amount of at least one peptidomimetic, or a pharmaceutically acceptable salt, stereoisomer, tautomer, hydrate, and/or solvate thereof. For example, the peptidomimetic can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

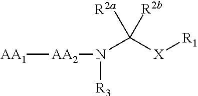

wherein,
AA₁ is selected from

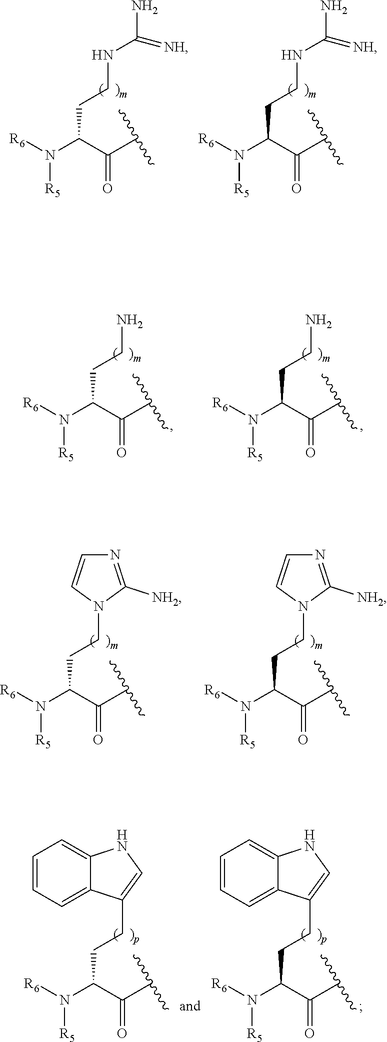

AA₂ is selected from

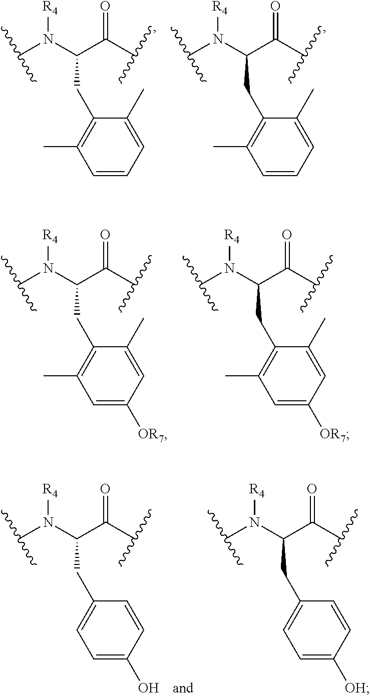

R₁ is selected from

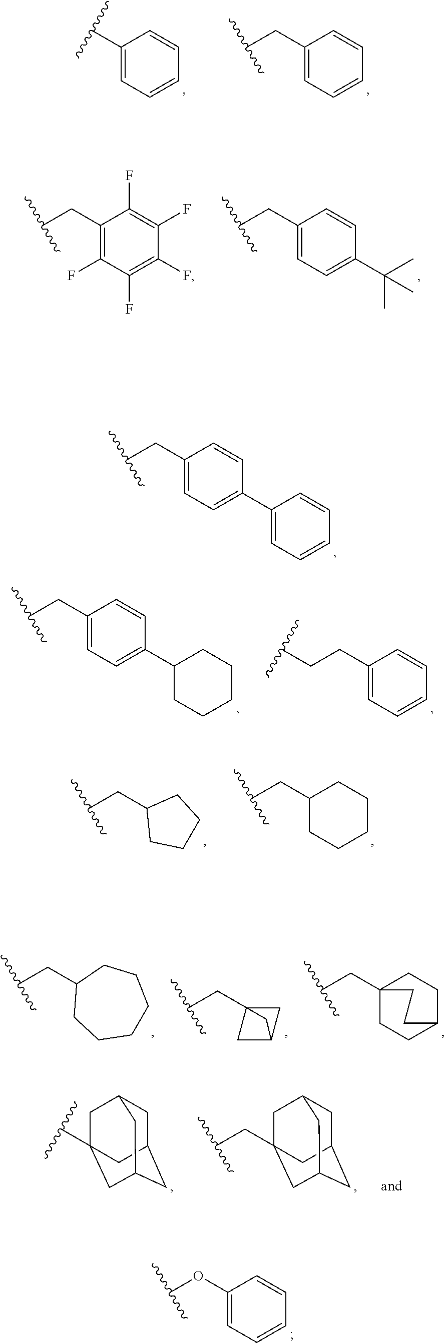

R^{2a} is selected from

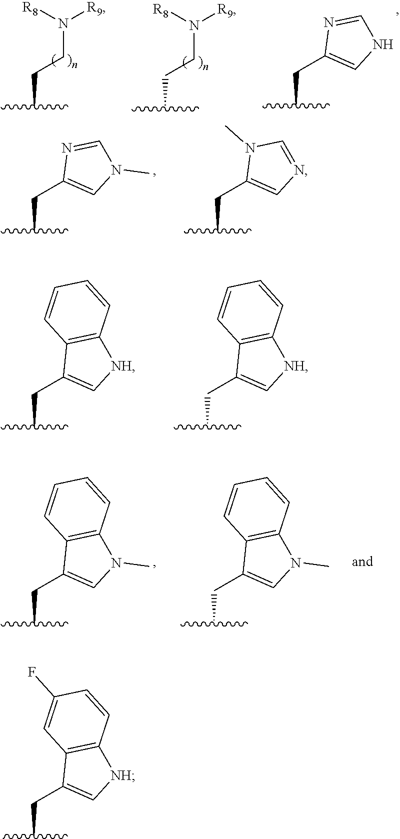

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

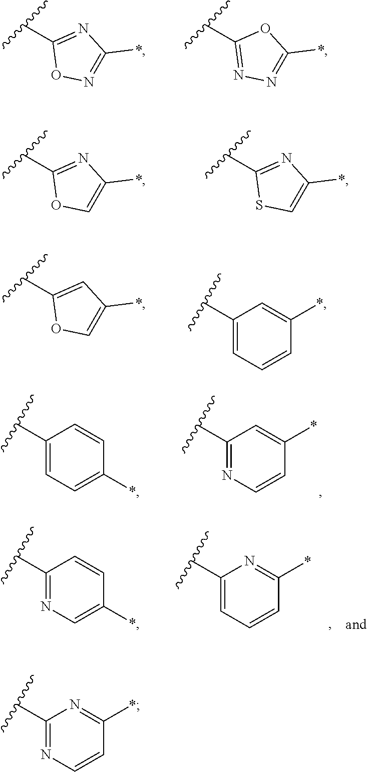

and
* denotes the point of attachment of X to R₁, and wherein one or more of the hydrogen atoms of the peptidomimetic is optionally substituted with a deuterium or fluorine atom.

In one aspect, the present disclosure provides a formulation or medicament for treating, preventing, inhibiting, ameliorating or delaying the onset of: (i) an ophthalmic disease, disorder condition; or (ii) deterioration of ellipsoid zone integrity in one or more eyes in a mammalian subject in need thereof, said formulation or medicament comprising a therapeutically effective amount of at least one peptidomimetic, or a pharmaceutically acceptable salt, stereoisomer, tautomer, hydrate, and/or solvate thereof. For example, the peptidomimetic used in the formulation can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (i.e. Formula II), or a pharmaceutically acceptable salt (e.g. (Formula IIa), stereoisomer, tautomer, hydrate, and/or solvate thereof. In some embodiments, the peptidomimetic is a peptidomimetic of Formula I, or a pharmaceutically acceptable salt, tautomer, hydrate, and/or solvate thereof:

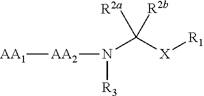

wherein,
AA₁ is selected from

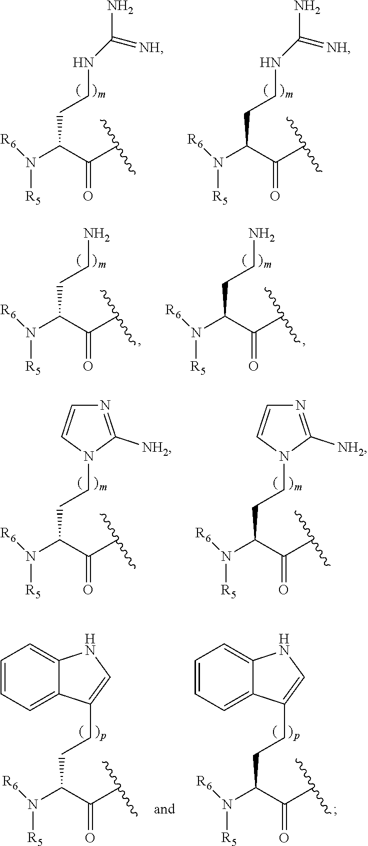

AA₂ is selected from

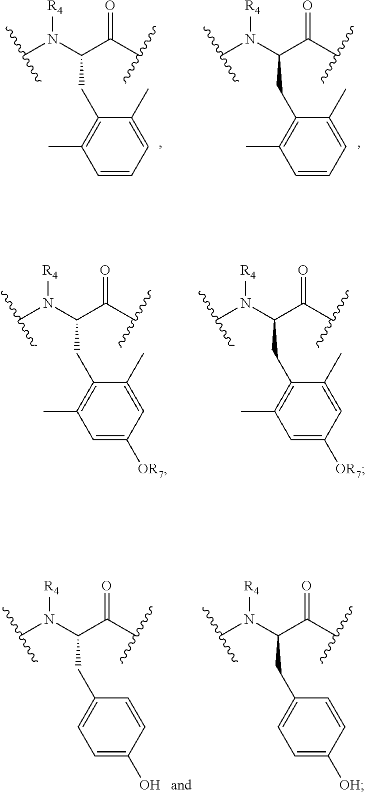

R₁ is selected from

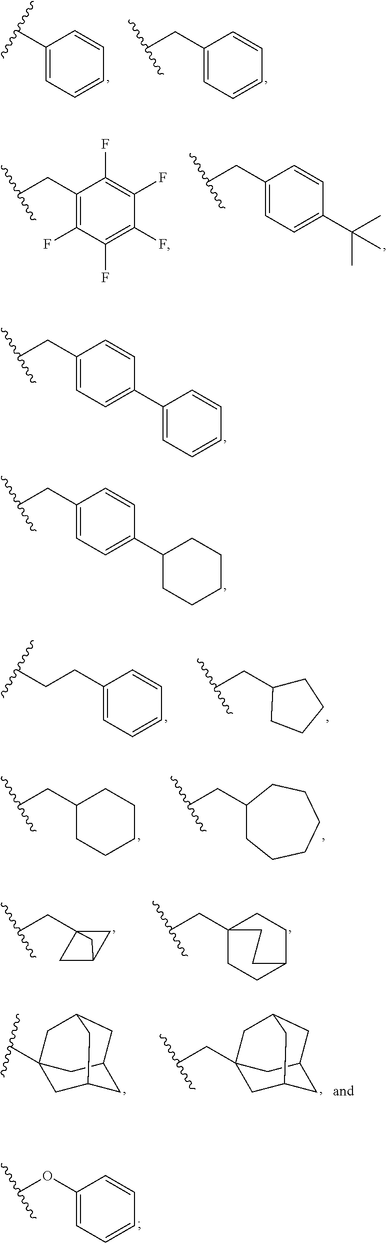

R^{2a} is selected from

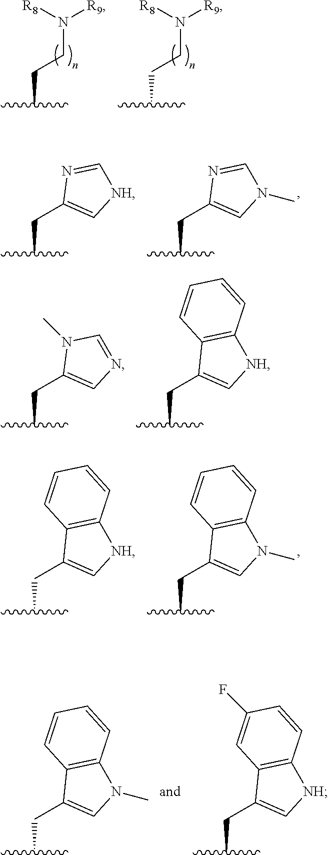

R^{2b} is H or CH₃;
R₃ and R₄ are independently selected from H and (C₁-C₆)alkyl;
R₅ and R₆ are independently H, methyl, ethyl, propyl, cyclopropyl, or cyclobutyl; or R₅ and R₆ together with the N atom to which they are attached form a 4-6-membered heterocyclyl;
R₇ is selected from H, (C₁-C₆)alkyl, cycloalkyl, and aryl;
R₈ and R₉ are independently selected from H, (C₁-C₆)alkyl, cycloalkyl, and aryl; or R₈ and R₉ together with the N atom to which they are attached form a 4-6-membered heterocyclyl;
m is 1, 2, or 3;
n is 1, 2, or 3;
p is 0 or 1;
X is selected from

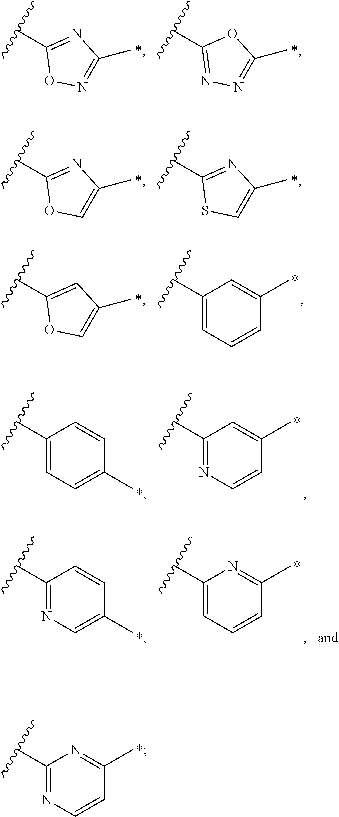

and
* denotes the point of attachment of X to R₁, and wherein one or more of the hydrogen atoms of the peptidomimetic is optionally substituted with a deuterium or fluorine atom.

### Determination of the Biological Effect of the Peptidomimetic-Based Therapeutic:

In various embodiments, suitable in vitro or in vivo assays can be performed to determine the effect of a specific peptidomimetic-based therapeutic and whether its administration is indicated for treatment or prevention. In various embodiments, in vitro assays can be performed with representative cells of the type(s) involved in the subject's disorder, to determine if a given peptidomimetic-based therapeutic exerts the desired effect upon the cell type(s). Compounds for use in therapy or prevention can be tested in suitable animal model systems. Similarly, for in vivo testing, any of the animal model system known in the art can be used prior to administration to human subjects. In one embodiment, administration of a peptidomimetic of Formula I, II, III, IV, V, VI, VII, VIII, IX, X, XI, XII, XIII, XIV, XV or a pharmaceutically acceptable salt (e.g., (IIa)), stereoisomer, tautomer, hydrate, and/or solvate thereof to a subject exhibiting symptoms associated with an ophthalmic condition will cause an improvement in (or prevention, inhibition, amelioration, delay in the onset of) one or more of the diseases, disorders or conditions experienced by the subject.

The effect of the peptidomimetic-based therapeutic on the ophthalmic disease, disorder or condition in the subject can be determined by examination of one or more eyes of the subject. In some embodiments, such determination may be made using, for example, examination techniques such as measuring the best corrected visual acuity (BCVA) of the subject over time to determine if the subjects vision is stable, improving or deteriorating. In some embodiments, such determination may be made using, for example, examination techniques such as measuring the low luminance visual acuity (LLVA) of the subject over time to determine if the subjects vision is stable, improving or deteriorating. In some embodiments, such determination may be made using, for example, examination techniques involving the use of any of the various forms of optical coherence tomography (OCT; including SDOCT, (TD)OCT or SS-OCT or OCTA) of the subject over time to determine if the subjects vision is stable, improving or deteriorating. In some embodiments, these examinations are used to evaluate the structures of the external limiting membrane (ELM), Bruch's membrane (BM), ellipsoid zone (EZ), interdigitation zone (IZ) and the retinal pigment epithelium (RPE). Use of this technology (particularly the various forms of OCT) is/are capable of accessing EZ integrity and EZ-RPE alterations, and any deterioration thereof over time. In some embodiments, administration of a peptidomimetic of Formula I, II, III, IV, V, VI, VII, VIII, IX, X, XI, XII, XIII, XIV, XV or a pharmaceutically acceptable salt (e.g., (IIa)), stereoisomer, tautomer, hydrate, and/or solvate thereof to a subject exhibiting symptoms associated with an ophthalmic condition will cause an improvement in (or prevention, inhibition, amelioration, delay in the onset of) one or more of the diseases, disorders or conditions experienced by the subject, including in some case, deterioration of the ellipsoid zone integrity in the subject.

The data presented in Examples 1, 3 and 4 demonstrate that the compound of Formula II (specifically the salt form Formula IIa), accumulates in the eyes (including substructures of the eyes) in amounts that would be expected to be therapeutically effective. The data in Example 1 illustrates that the compound of Formula IIa accumulates in the eyes (and their substructures) of rabbits in greater concentrations than does elamipretide (whether administered topically or subcutaneously); elamipretide being a compound shown to be therapeutically active in recent P1 and P2 human clinical trials including a correlation associated with improved LLVA in combination with improved EZ integrity (See the Introduction, above). Both elamipretide and peptidomimetics of Formula I are mitochondria-targeted. Example 2 demonstrates that both elamipretide and the compound of Formula IIa exhibit similar beneficial effects of improving mitochondrial function in RPE cells derived from AMD donors. For these reasons, the peptidomimetics (e.g. Formula I, II, III, IV, V, VI, VII, VIII, IX, X, XI, XII, XIII, XIV, XV, or pharmaceutically acceptable salts thereof) are expected to be useful in treating, preventing, inhibiting, ameliorating or delaying the onset of ophthalmic diseases, disorders and conditions generally, including without limitation, GA, glaucoma and/or wet or dry age-related macular degeneration. Furthermore, it is anticipated, based on these results, that the administration of the peptidomimetics (e.g. Formula I, II, III, IV, V, VI, VII, VIII, IX, X, XI, XII, XIII, XIV, XV, or pharmaceutically acceptable salts thereof) will be useful in treating, preventing, inhibiting, ameliorating or delaying the onset of deterioration of the (mitochondria-rich) ellipsoid zone integrity in one or more eyes of a mammalian subject in need thereof.

### Animal Models:

Compounds for use in therapy can be tested in suitable animal model systems including, but not limited to, rats, mice, chicken, cows, monkeys, rabbits, and the like, prior to testing in human subjects. Similarly, for in vivo testing, any of the animal model systems known in the art can be used prior to administration to human subjects. In some embodiments, in vitro or in vivo testing is directed to the biological function of a compound of Formula (II), or a pharmaceutically acceptable salt (e.g., (IIa)), stereoisomer, tautomer, hydrate, and/or solvate thereof. In some embodiments, in vitro or in vivo testing is directed to the biological function of (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or pharmaceutically acceptable salt (e.g., (IIa)), stereoisomer, tautomer, hydrate, and/or solvate thereof. In some embodiments, the animal model is the Sprague Dawley rat.

### Modes of Administration and Effective Dosages:

Any method known to those in the art for contacting a cell, organ or tissue with a peptidomimetic of Formula (I), or a pharmaceutically acceptable salt, stereoisomer, tautomer, hydrate, and/or solvate thereof. In some embodiments, the cell, organ or tissue is contacted with (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or pharmaceutically acceptable salt (e.g., (Formula IIa)), stereoisomer, tautomer, hydrate, and/or solvate thereof. Suitable methods include in vitro, ex vivo, or in vivo methods. In vivo methods typically include the administration of a peptidomimetic to a mammal, such as a human. When used in vivo for therapy, the peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or a pharmaceutically acceptable salt (e.g., Formula IIa), stereoisomer, tautomer, hydrate, and/or solvate thereof, can be used. The dose and dosage regimen will depend upon the degree of the disease, disorder or condition in the subject, the characteristics of the particular peptidomimetic used, e.g., its therapeutic index, the subject, and the subject's history.

The effective amount may be determined during pre-clinical trials and clinical trials by methods familiar to physicians and clinicians. An effective amount of a peptidomimetic useful in the methods may be administered to a mammal in need thereof by any of a number of well-known methods for administering pharmaceutical compounds. For example, the peptidomimetic may be administered subcutaneously, intravitreally, topically, intraocularly, ophthalmically, orally, intranasally, systemically, intravenously, intraperitoneally, intradermally, intrathecally, intracerebroventricularly, iontophoretically, transmucosally, or intramuscularly.

The peptidomimetic may be formulated as a pharmaceutically acceptable salt. The term “pharmaceutically acceptable salt” is a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide or peptidomimetic contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri- (tris-) acid salt), a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a tri- (tris-) hydrochloride salt (e.g., Formula IIa), a mono-tosylate salt, a bis-tosylate salt, or a tri-tosylate salt. In some embodiments, the peptidomimetic is formulated as a mono-HCl, bis-HCl salt or a tri- (or tris)-HCl salt (e.g., Formula IIa).

The peptidomimetics described herein, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or a pharmaceutically acceptable salt (e.g., Formula IIa), stereoisomer, tautomer, hydrate, and/or solvate thereof can be incorporated into pharmaceutical compositions (e.g., a formulation or medicament) for administration, singly or in combination, to a subject for the treatment or prevention of a disease, disorder or condition described herein. The peptidomimetic may be formulated with other compounds such as a therapeutic agent, a peptide, another peptidomimetic or mixtures thereof. In some embodiments of the methods of the present technology, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), or a pharmaceutically acceptable salt (e.g., Formula IIa), stereoisomer, tautomer, hydrate, and/or solvate thereof. Such pharmaceutical compositions typically include the active agent and a pharmaceutically acceptable carrier. In some embodiments, the pharmaceutical compositions can be used as medicaments or in the preparation of medicaments for administration to a subject suffering from an ophthalmic condition or disease. Pharmaceutically acceptable carriers include saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions (e.g., a formulation or medicament) can be formulated to be compatible with its intended route of administration. Examples of routes of administration include parenteral (e.g., intravenous, intradermal, intraperitoneal or subcutaneous), oral, systemic, intravitreal, inhalation, transdermal (topical), intraocular, ophthalmic, intrathecal, intracerebroventricular, iontophoretic, transmucosal, intravitreal and intramuscular administration. In some embodiments, the route of administration is oral. In some embodiments, the route of administration is subcutaneous. In some embodiments, the route of administration is topical. In some embodiments, the route of administration is intraocular. In some embodiments, the route of administration is ophthalmic.

Solutions or suspensions (e.g., a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 7 days or more of treatment).

Pharmaceutical compositions (e.g., a formulation or medicament) suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). A composition for administration by injection will generally be sterile and should be fluid to the extent that easy syringability exists. It should be stable under the conditions of manufacture and storage and must be preserved against the contaminating action of microorganisms such as bacteria and fungi.

The peptidomimetic containing compositions (e.g., a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions (e.g., a formulation or medicament) can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions (e.g., formulations or medicaments) generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

One may dilute or increase the volume of a formulation or medicament comprising a compound, therapeutic agent, peptide, peptidomimetic or mixtures thereof with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation or medicament comprising compound, therapeutic agent, peptide, peptidomimetic or mixtures thereof with an inert material into a solid dosage form. Materials used as disintegrates include but are not limited to starch, including the commercial disintegrant based on starch, e.g., Explotab®. Sodium starch glycolate, Amberlite®, sodium carboxymethylcellulose, ultramylopectin, sodium alginate, gelatin, orange peel, acid carboxymethyl cellulose, natural sponge and bentonite may all be used as disintegrants. Another form of the disintegrants are the insoluble cationic exchange resins. Powdered gums may be used as disintegrants and as binders, and these can include powdered gums such as agar, karaya or tragacanth. Alginic acid and its sodium salt are also useful as disintegrants.

Binders may be used to hold a compound, therapeutic agent, peptide, peptidomimetic or mixtures thereof in a formulation with an inert material together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the formulation.

An anti-frictional agent may be included in the formulation or medicament comprising a compound, therapeutic agent, peptide, peptidomimetic or mixtures thereof to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of a compound, therapeutic agent, peptide, peptidomimetic or mixtures thereof into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or medicament comprising a compound, therapeutic agent, peptide, peptidomimetic or mixtures thereof of the technology or derivative either alone or as a mixture in different ratios.

Pharmaceutical preparations (e.g., a formulation or medicament) which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations or medicaments for oral administration should be in dosages suitable for such administration.

For administration of a formulation, medicament or compound by inhalation for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

A compound, composition (e.g., formulation or medicament), therapeutic agent, peptide, peptidomimetic or mixtures thereof can be delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., AnnalIntMed 3:206-212 (1989) (antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995, to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

For ophthalmic or intraocular formulations, any suitable mode of delivering the peptidomimetics, such as a peptidomimetic of Formula I, or a pharmaceutically acceptable salt, tautomer, hydrate, and/or solvate thereof (with or without therapeutic agents, peptides or other peptidomimetics), to the eye or regions near the eye can be used. For example, the peptidomimetic can be (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of formulations suitable for administration in or near the eye include, but are not limited to, ocular inserts, minitablets, and topical formulations such as eye drops, ointments, and in situ gels. In one embodiment, a contact lens is coated with a peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof. In some embodiments, a single dose comprises from between 0.1 ng to 5000 g, 1 ng to 500 g, or 10 ng to 100 g of the peptidomimetics administered to the eye.

Eye drops can comprise a sterile liquid formulation that can be administered directly to the eye. In some embodiments, eye drops comprising one or more peptidomimetics described herein, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof can be used and may further comprise one or more preservatives. In some embodiments, the optimum pH for eye drops equals that of tear fluid and is about 7.4.

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, silicon containing formulations and cellulose acetate phthalate. In some embodiments, the compound, therapeutic agent, peptide, peptidomimetic or mixtures thereof is formulated into an in-situ gel (as the pharmaceutical composition).

For topical ophthalmic administration, a compound, therapeutic agent, peptide, peptidomimetic or mixtures thereof may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more peptidomimetics, slow release of one or more peptidomimetics with constant speed and limiting of one or more peptidomimetics' systemic absorption. In some embodiments, an ocular insert comprises one or more peptidomimetics described herein, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers.

Minitablets are biodegradable, solid drug forms, that transit into gels after application to the conjunctival sac, thereby extending the period of contact between active ingredient and the eyeball surface, which in turn increases the active ingredient's bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more peptidomimetics described herein, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol or Carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more peptidomimetics described herein, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

Systemic administration of a compound, composition (e.g., formulation or medicament), therapeutic agent, peptide, peptidomimetic or mixtures thereof, as described herein, can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A compound, composition (e.g., formulation or medicament), therapeutic agent, peptide, peptidomimetic or mixtures thereof can be formulated in a carrier system. The carrier can be a colloidal system. The colloidal system can be a liposome, a phospholipid bilayer vehicle. In one embodiment, the compound, composition (e.g., formulation), therapeutic agent, peptide, peptidomimetic or mixtures thereof is encapsulated in a liposome while maintaining integrity of the compound, therapeutic agent, peptide, peptidomimetic or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the compound, composition (e.g., formulation), therapeutic agent, peptide, peptidomimetic or mixtures thereof can be embedded in the polymer matrix, while maintaining integrity of the composition. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the compound, composition (e.g., formulation), therapeutic agent, peptide, peptidomimetic or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

In addition to the formulations described above, compound, compositions, therapeutic agent, peptide, peptidomimetic or mixtures thereof may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

A compound, composition, therapeutic agent, peptide, peptidomimetic or mixtures thereof may be provided in particles or polymer microspheres. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The compounds, compositions, therapeutic agents, peptides, peptidomimetics or mixtures thereof also may be dispersed throughout the particles. The compounds, compositions, therapeutic agents, peptides, peptidomimetics or mixtures thereof also may be adsorbed into the particles. The particles may be of any order release kinetics, including zero-order release, first-order release, second-order release, delayed release, sustained release, immediate release, and any combination thereof, etc. The particle may include, in addition to the compounds, compositions, therapeutic agents, peptides, peptidomimetics or mixtures thereof, any of those materials routinely used in the art of pharmacy and medicine, including, but not limited to, erodible, nonerodable, biodegradable, or nonbiodegradable material or combinations thereof. The particles may be microcapsules which contain the compound of the technology in a solution or in a semi-solid state. The particles may be of virtually any shape.

Both non-biodegradable and biodegradable polymeric materials can be used in the manufacture of particles for delivering the compounds, compositions, therapeutic agents, peptides, peptidomimetics or mixtures thereof. Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and polycaprolactone.

The compounds, compositions, therapeutic agents, peptides, peptidomimetics or mixtures thereof may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant (depot) is constructed and arranged to deliver therapeutic levels of the active ingredient (i.e. compound, therapeutic agent, peptide, peptidomimetic or mixtures thereof) for at least 7 days, for at least 30 days, for at least 60 days, for at least 90 days, for at least 120 days, for at least 180 days or for at least 365 days. In some embodiments, the “long-term” release means 30-60 days, 60-90 days, 90-120 days, 120-180 days, or 180-365 days. Long-term sustained release implants are well-known to those of ordinary skill in the art and include some of the release systems described above.

Dosage, toxicity and therapeutic efficacy of any compounds, compositions (e.g., formulations), therapeutic agents, peptides, peptidomimetics or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the peptidomimetics, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 0.5-1 mg/kg body weight or 1-10 mg/kg body weight every day, every two days or every three days or within the range of 1-10 mg/kg every week, every two weeks or every three weeks. In one embodiment, a single dosage of peptide or peptidomimetic ranges from 0.001-10,000 micrograms per kg body weight. In one embodiment, mitochondria-targeting peptidomimetic concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of a peptidomimetic may be defined as a concentration of peptidomimetic at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the compounds, therapeutic agents, peptides, peptidomimetics or mixtures thereof described herein can include a single treatment or a series of treatments.

### Combination Therapies:

In some embodiments, the peptidomimetics, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof may be combined with one or more additional therapeutic agents for the prevention or treatment of ophthalmic conditions or disease. In some embodiments of the methods of the present technology, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof. In some embodiments, additional therapeutic agents include, but are not limited to, administration of carbachiol (Carbastat® or Carboptic®), Polocarpine (Salagen®), timolol (Timoptic®), betaxolol (Betoptic® or Keflone®), Carteolol (Cartrol® or Ocupress®), Levobunolol (Liquifilm®), brimonidine (Lumify® or Mirvaso®), apraclonidine (Iopidine®), latanoprost (Xalantan®), travoprost (Travatan®), bimatoprost (Lumigan®), talfluprost (Taflotan®), unoprostone isopropyl (Rescula®), dorzolamide (Trusopt®), brinzolamide (Azopt®), acetazolamide (Diamox®), methazolamide (Neptazane®), brimonidine tartrate/timolol maleate (Combigan®), timolo-dorzolamide (Cosopt®), travoprost-timolol (DuoTrav®) and latanoprost and timolol maleate (Xalacom®).

In some embodiments, the peptidomimetics, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof may be combined with one or more additional therapeutic agents (alone or in a formulation) selected from: an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, or a carotenoid. In some embodiments, the additional therapeutic agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations of two or more of the foregoing.

In some embodiments, any one of the foregoing additional therapeutic agents is administered separately, simultaneously, or sequentially with the mitochondria-targeting peptidomimetic(s). In some embodiments, the dose of additional therapeutic agent is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of resveratrol is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the additional therapeutic agent is administered twice per day, daily, every 48 hours, every 72 hours, twice per week, once per week, once every two weeks, once per month, once every 2 months, once every 3 months, or once every 6 months. In some embodiments, the dose of additional therapeutic agent is dependent upon the subject's weight and/or age.

In one embodiment, an additional therapeutic agent is administered to a subject in combination with at least one peptidomimetic, such that a synergistic therapeutic effect is produced. For example, administration of at least one peptidomimetic with one or more additional therapeutic agents for the prevention or treatment of an ophthalmic condition or disease will have greater than additive effects in the prevention or treatment of the condition or disease. Therefore, lower doses of one or more of any individual therapeutic agent may be used in treating or preventing an ophthalmic condition or disease resulting in increased therapeutic efficacy and decreased side-effects.

In some embodiments, multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

In some embodiments, the peptidomimetics, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., (Formula IIa)), stereoisomer, tautomer, hydrate, and/or solvate thereof may be combined with one or more additional therapeutic techniques including gene therapy for the prevention or treatment of diseases, such as, for example, ophthalmic monogenic disorders. Accordingly, in some embodiments, the peptidomimetics, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Formula II), a pharmaceutically acceptable salt (e.g., Formula IIa), stereoisomer, tautomer, hydrate, and/or solvate thereof may be administered to a subject in combination with gene therapy.

### EXAMPLES

The present technology is further illustrated by the following examples, which should not be construed as limiting in any way.

### Example 1—Comparison of the Uptake of Elamipretide and Compound of Formula IIa in Plasma and Various Compartments of the Eye in a Rabbit Model

### A. Test Articles:

(i) elamipretide
(ii) compound of Formula IIa

### B. Formulation for Various Groups Studied:

(i) Groups 1 & 3: Subcutaneous administration of elamipretide (corrected for potency) or compound of Formula IIa

3 mg/mL elamipretide or compound of Formula IIa in saline was dosed subcutaneously at 1.5 mg/kg. A 3 mg/mL elamipretide or compound of Formula IIa formulation was prepared by dissolving 81 mg elamipretide in a total volume of 27 mL of sterile saline and mixing well.
(ii) Groups 2 & 4: Topical Ocular Formulation for administration of elamipretide or compound of Formula IIa

For each preparation (i.e., elamipretide or compound of Formula IIa, sodium chloride (105 mg) was dissolved in 16 mL of water for injection with mixing. With mixing, 82.8 mg of sodium phosphate monobasic, monohydrate was then added. With mixing, 0.400 mL of a 5 mg/mL benzalkonium chloride solution in water (to be prepared by diluting 0.100 mL of a 50% benzalkonium chloride solution in water to 10 mL with water for injection) was then added. Two hundred (200) mg of elamipretide or compound of Formula IIa was then added with mixing and the formulation pH was adjusted to 5.8 (+/−0.1) with 1 M sodium hydroxide in water solution. The final volume of the formulation was then be brought to 20 mL using water for injection. Each formulation (elamipretide or compound of Formula IIa) was used for topical ocular administration at a dose of 50 μL/eye OU.

In all cases, the formulation was administered on the day of its preparation.

### C. Animals and Number of Animals:

A total of 48 male Dutch Belted Rabbits were used in the study. Each animal was approximately 2 kg.

### D. Husbandry:

Animals were individually housed in compliance with all applicable laws, regulations and guidelines. No other species was kept in the same room. The animals were exposed to 12 hours light/12 hours dark as the light cycle, except during the dark cycle when the lights will be turned on to perform any study related activities. Room temperature was kept between 16 to 22° C. and relative humidity was kept between 30 to 70%.

All animals had access to rabbit chow on an ad libitum basis. Water was available ad libitum to each animal via a water bottle with sipper tube. Animals were acclimated to their housing for at least 5 days following their receipt into the facility prior to their first day of dosing.

### E. Prestudy Health & Care of Animals:

An examination was performed on both eyes of all study animals by the vendor prior to shipping so that the eyes were free from any abnormality or defect. All animals received for this study were assessed as to their general health. During acclimation, each animal was observed for any abnormalities or for the development of infectious disease.

All animals were treated in accordance with the study protocol. Procedures in the protocol were approved by the Institutional Animal Care and Use Committee (IACUC) and complies with acceptable standard animal welfare and humane care.

TABLE 1
Summarizing Study Design:

| | | Dose | PK | |
|---|---|---|---|---|
| | | level/ | Terminal | |
| | Test Article/ | Dose | Timepoints | Fluid and |
| Group | Route | Frequency | (Postdose) | Tissues |
| 1 | Elamipretide | 3 mg/mL | 0.5, 1, 2, 4, | Plasma, retina, |
| (n = 12) | Sub- | Single | 8, and 24 | and optic nerve |
| | cutaneous | dose | hours | |
| 2 | Elamipretide | 20 mg/mL | 0.5, 1, 2, 4, | Plasma, aqueous |
| (n = 12) | Ocular | BIDª | 8, and 24 | humor, retina, |
| | Topical | | hours | conjunctiva, |
| | (both eyes) | | | cornea, sclera, |
| | | | | and optic nerve |
| 3 | Formula IIa | 3 mg/mL | 0.5, 1, 2, 4, | Plasma, retina, |
| (n = 12) | Sub- | Single | 8, and 24 | and optic nerve |
| | cutaneous | dose | hours | |
| 4 | Formula IIa | 20 mg/mL | 0.5, 1, 2, 4, | Plasma, aqueous |
| (n = 12) | Ocular | BID^{a} | 8, and 24 | humor, retina, |
| | Topical | | hours | conjunctiva, |
| | (both eyes) | | | cornea, sclera, |
| | | | | and optic nerve |

^{a}Doses were administered to both eyes (i.e., OU), every 8 to 12 hours for 11 total doses.

### G. Test Article Administration/Dosing:

For subcutaneous administration, rabbits were dosed via a single subcutaneous injection of 1.5 mg/kg and at the scruff of the neck between the shoulder blades using a syringe with an attached 27G×½ inch needle (or similar). The site of injection was checked to ensure no leakage of dose immediately following administration.

For topical administration, a calibrated Gilson positive displacement pipette was used to administer 50 μL of formulation onto the globe the eye while the lower eyelid was pulled away from the globe. Administration was twice daily (i.e., BID). Doses were administered to both eyes, every 8 to 12 hours for 11 total doses.

### H. Terminal Procedures:

Animals were euthanized by barbiturate overdose at the designated timepoints or as needed for humane reasons.

Terminal blood samples (approximately 6 mL) were collected from two animals/group/timepoint via the central ear artery at approximately 0.5, 1, 2, 4, 8, and 24 hours postdose. Blood samples were collected into tubes containing K₂EDTA as the anticoagulant and inverted several times to ensure adequate mixing of blood and anticoagulant and placed on ice. Within 30 min of collection, samples were centrifuged to harvest plasma and stored at −80° C. until analyzed.

Following blood collection, designated animals were euthanized by barbiturate overdose. Following euthanasia, both eyes of each rabbit were harvested and dissected for collection of ocular tissue and fluid. Optic nerve and retina were collected from Group 1 and 3 animals, and aqueous humor, retina, conjunctiva, cornea, sclera, and optic nerve were collected from Group 2 and 4 animals. Following dissection, all fluids and tissues were placed in pre-tared tubes, weight collected, and then flash frozen on dry ice and placed in a freezer at −80° C. or lower until analyzed. Concentrations of test article in plasma in the ocular tissues (ng/g tissue) and fluids (ng/mL fluid) were determined utilizing a method previously developed by the CRO.

### I. Results:

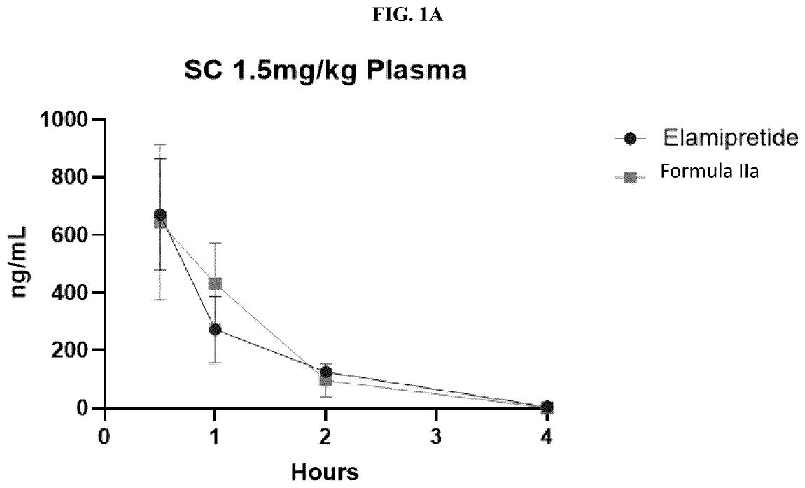

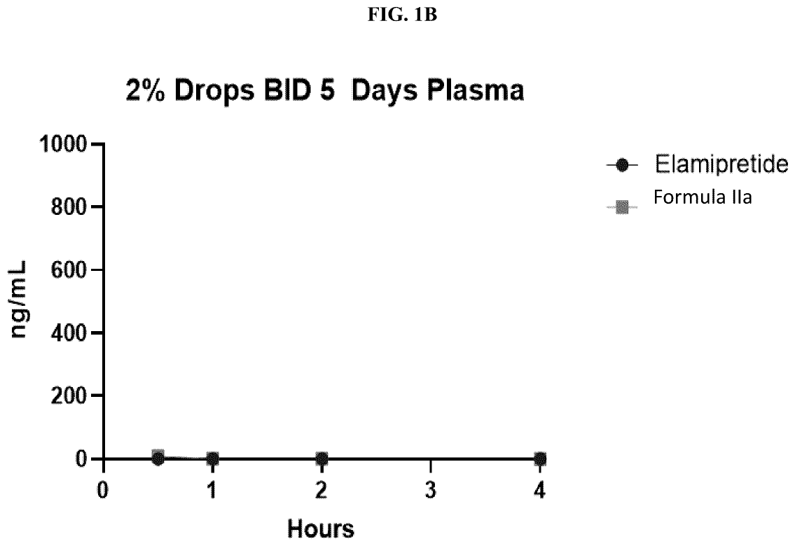

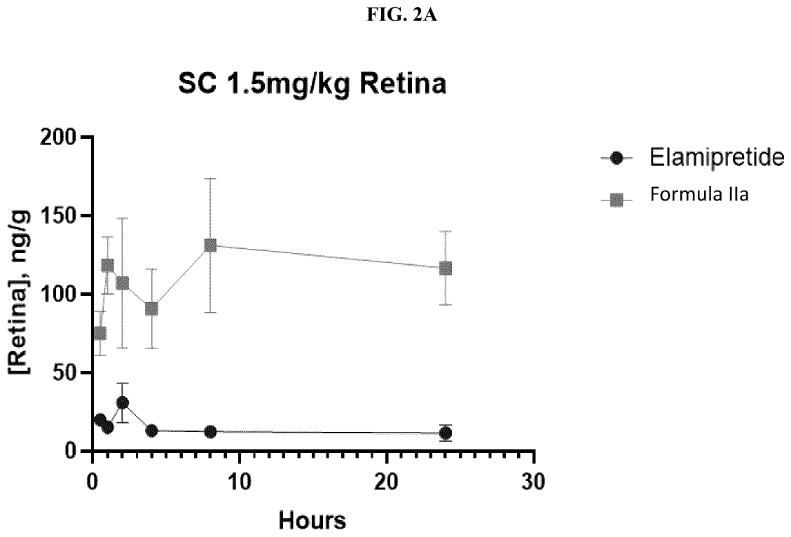

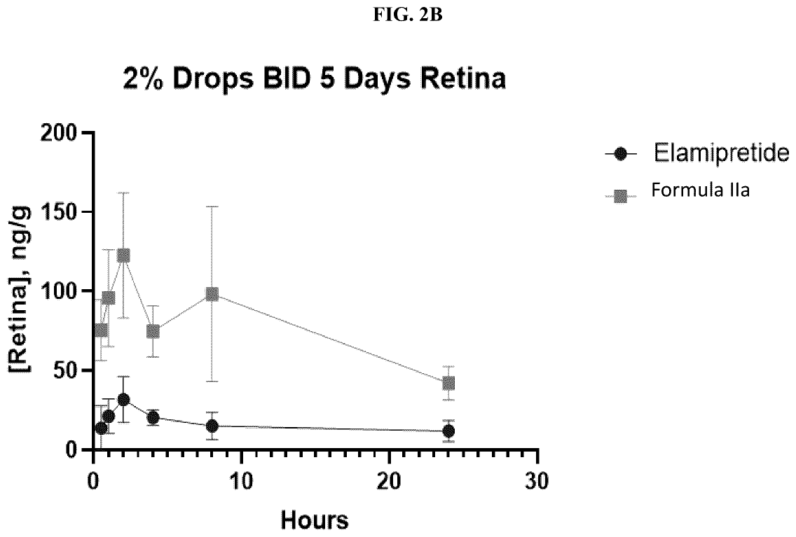

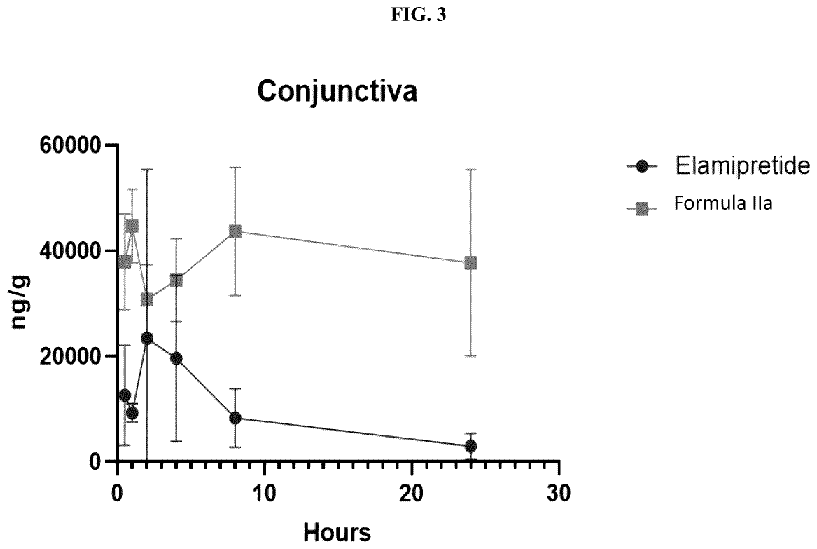

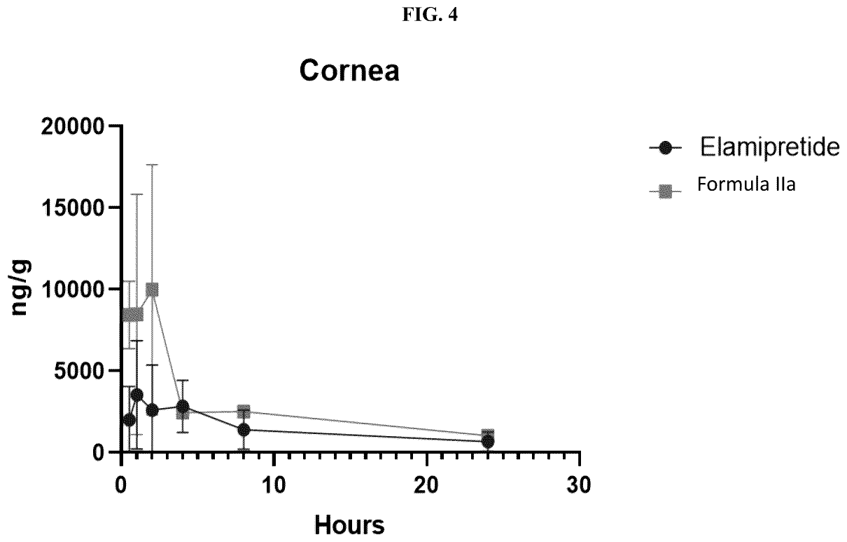

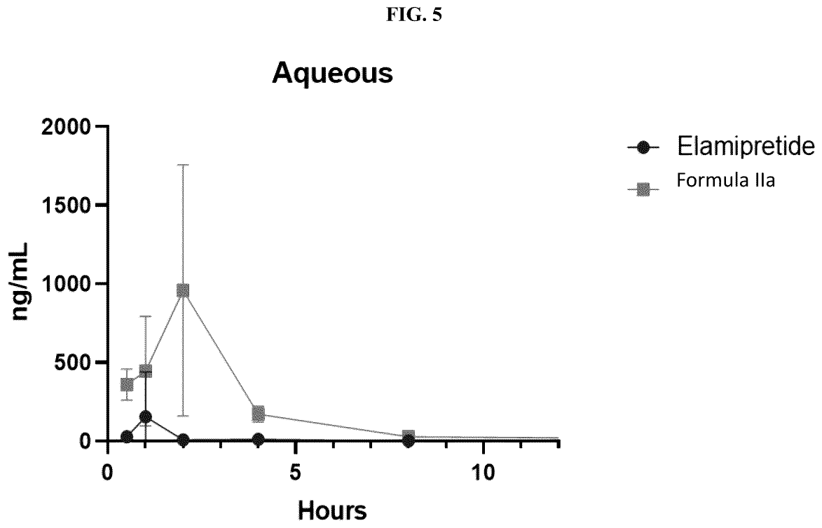

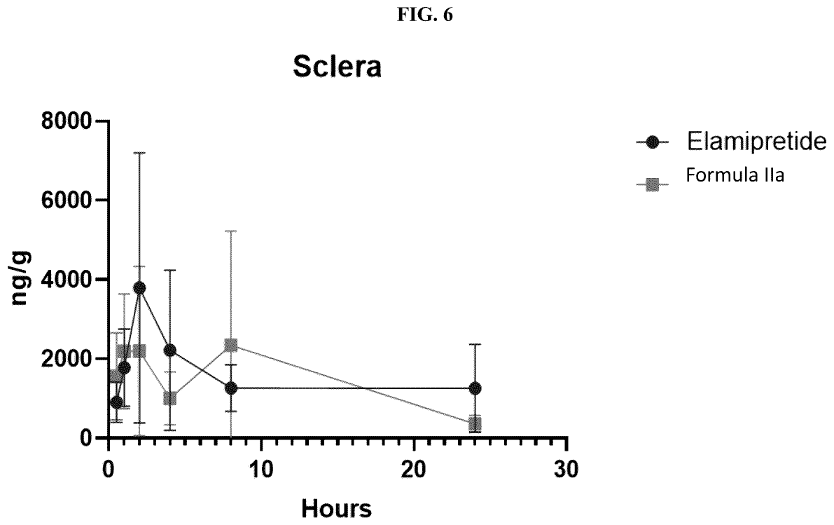

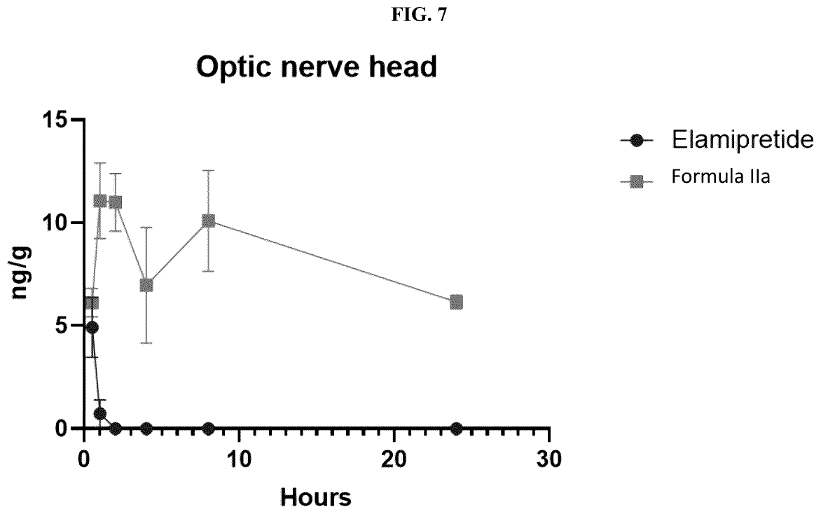

The results are illustrated graphically in FIGS. 1A to 7. With reference to FIGS. 1A and 1B the concentration of elamipretide and compound of Formula IIa in plasma is roughly equivalent regardless of the mode of administration. With reference to FIGS. 2A and 2B, however, the concentration of elamipretide that has accumulated in the retina is much lower than the concentration of compound of Formula IIa, regardless of the mode of administration (i.e., topical or subcutaneous). With reference to FIGS. 3 to 7 (all of which are for topical administration only), with the exception of the sclera (FIG. 6), in all cases the concentration of compound of Formula IIa is higher than elamipretide in the eye tissue examined.

### J. Summary:

The data illustrates that compound of Formula IIa generally accumulates in the plasma of the rabbit in roughly equivalent concentrations as compared with elamipretide whether dosing topically or by subcutaneous injection. However, in various of the eye tissues (e.g., retina, conjunctiva, cornea, aqueous humor, and optic nerve head) compound of Formula IIa accumulates in higher concentration than does elamipretide whether administered subcutaneously or topically (i.e. via eye-drops).

### Example 2—Efficacy of Elamipretide and Compound of Formula IIa in an iPSC-Derived RPE Preclinical Model of Dry AMD

### Introduction:

Age-related macular degeneration (AMD) is characterized by changes in Bruch's membrane followed by dysfunction and atrophy of retinal pigment epithelial (RPE) cells, which is a key feature of AMD pathogenesis. Somatic cells harvested from AMD patients can be reprogrammed to form RPE and model patient-specific disease. This example combines the use of an in vitro model for age-related changes to Bruch's membrane with induced pluripotent stem cell (iPSC)-derived RPE cells from patients with AMD (as described in Gong et al. STEM CELLS Transl Med. 9:364-376 (2020), and briefly described below), and demonstrates the efficacy of elamipretide and compound of Formula IIa in methods for treating, preventing, inhibiting, amelioration or delaying the onset of dry AMD.

### Methods:

General. iPSC-derived RPE were generated from AMD patients (2 atrophic; 1 exudative) and patients with no history of AMD (n=3). To test the therapeutic efficacy of elamipretide and compound of Formula IIa, cell viability was analyzed on nitrite-modified extracellular matrix (ECM), a typical modification of aged Bruch's membrane, for 24 hrs. DNA microarrays were used to elucidate gene expression in AMD-derived RPE cultured on nitrite-modified ECM.

Primary fibroblast culture. Fibroblasts from AMD patients and patients with no history of AMD were isolated as described in Fields et al., PLoS One 12:e0177763 (2017). Details on the patients are provided in Table 2. Cultures were obtained in Dulbecco's Modified Eagle Medium (DMEM; Thermo Fisher Scientific, Waltham, MA) containing 10% fetal bovine serum (FBS; Thermo Fisher Scientific) and cultured in a humidified 37° C., 5% CO₂ incubator.

TABLE 2
Patient Donor Demographics

| RPE Cell | | Donor Age | | | Reprograming |
|---|---|---|---|---|---|
| Line | Eye History | (years) | Gender | Source | Method |
| AMD 1 | Atrophic AMD with GA | 89 | Female | Fibroblast | mRNA |
| AMD 2 | Bilateral exudative AMD | 74 | Male | Fibroblast | mRNA |
| AMD 3 | Early Atrophic AMD | 65 | Female | Fibroblast | mRNA |
| | some with GA | | | | |
| Normal 1 | No history of AMD | 76 | Male | Fibroblast | mRNA |
| Normal 2 | No history of AMD | 65 | Male | Fibroblast | mRNA |
| Normal 3 | No history of AMD | 60 | Female | Fibroblast | mRNA |

AMD, age-related macular degeneration;
GA, geographic atrophy;
RPE, retinal pigment epithelium

Feeder free and non integration reprogramming. Fibroblasts were grown to 5×10⁴ cells/well and then treated with modified messenger ribonucleic acid (mRNA) encoding reprogramming factors, octamer-binding transcription factor 3,4 (Oct3/4), SRY (sex determining region Y)-box 2 (Sox2), Kruppel-like factor 4 (Klf4), c-Myc, NANOG homeobox protein (NANOG), and Lin-28 homolog A(Lin-28) using the fully automated platform New York Stem Cell Foundation (NYSCF) Research Institute Global Stem Cell Array as described in Paull et al., Nat Methods 12:885-892 (2015) or using the Stemgent StemRNA 3rd Gen Reprogramming Kit (REPROCELL USA Inc., Beltsville, MD, www.reprocell.com) according to the manufacturer's protocol. iPSC cultures were expanded by passaging every 5-7 days using Accutase (Sigma-Aldrich, St. Louis, MO, www.sigmaaldrich.com) and cultured for use in downstream experiments.

Immunofluorescence. After differentiation, iPSC-derived RPE cell lines were fixed and stained as described in Fields et al. (2017). Exemplary antibodies are provided in Table 3. Cell nuclei were labeled with 4′,6-diamidino-2-phenylindole (DAPI; Sigma-Aldrich). Cells were visualized by a Zeiss LSM 800 confocal laser scanning microscope with the use of Zen microscope software (Carl Zeiss, Oberkochen, Germany, www.zeiss.com).

TABLE 3
List of Antibodies Used for iPSC and RPE Cell Markers

| | | Catalog | |
|---|---|---|---|
| Antibody | Company | Number | Target Cells |
| ZO-1 | Thermo Fischer Scientific, | 33-9100 | RPE cell |
| | Waltham MA | | (tight junction) |
| Na—K | Thermo Fischer Scientific, | MA5-32184 | RPE cell |
| ATPase | Waltham MA | | |
| RPE65 | Novus Biologicals, | NB100-355 | RPE cell |
| | Littleton, CO | | |

iPSC, induced pluripotent stem cell;
RPE, retinal pigment epithelium;
OCT4, octamer-binding transcription factor 4;
Sox2, SRY (sex determining region Y)-box 2;
SSEA-4, stage-specific embryonic antigen 4;
TRA-1-60, keratin sulphate-associated antigens-1-60;
ZO-1, zonula occludens-1;
Na—K ATPase, sodium potassium ATPase;
RPE65, retinal pigment epithelium-specific 65 kDa protein.

Differentiation of human iPSCs into RPE cells. Human iPSC-derived RPE cell lines were differentiated as described in Fields et al. (2017) and Gong et al., PLoS One 10:e0143272 (2015). Patches of pigmented iPSC-derived RPE cells were micro-dissected and plated onto laminin-coated plates until confluent. Cell cultures were maintained in RPE cell differentiation medium and allowed to form monolayers.

Preparation of RPE cell-derived ECM and nitrite-modified ECM RPE cell-derived ECM plates were prepared from ARPE-19 cells as described in Wang et al., Curr Eye Res. 30:691-702 (2005); Fields et al. (2017); and Moreira et al., Transl Vis Sci Technol. 4:10 (2015). ECM on 96-well plates were used to create two experimental plating surfaces (nontreated ECM and nitrite-modified ECM). Nitrite-modified ECM was prepared by adding 100 mM sodium nitrite to ECM followed by incubation at 37° C. for 7 days. Plates were then washed with DPBS and incubated with DPBS for 4 hours to completely remove the nitrite.

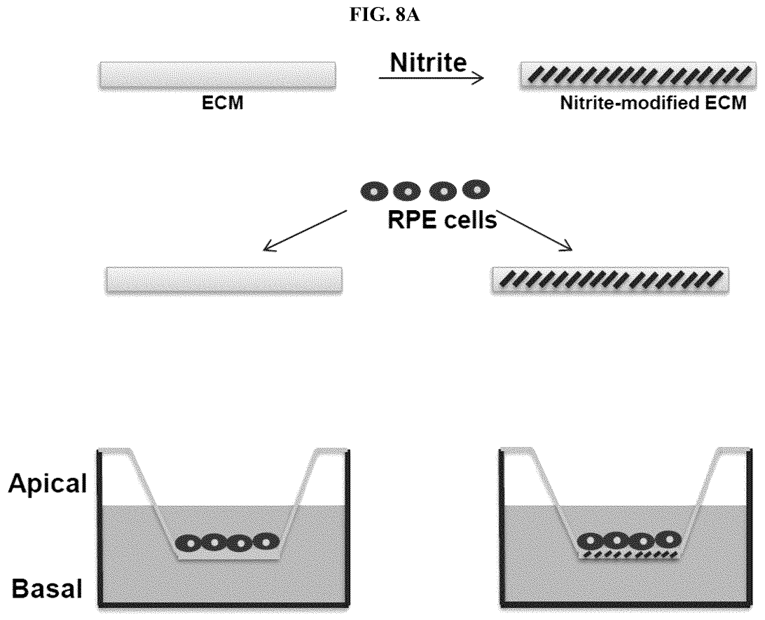

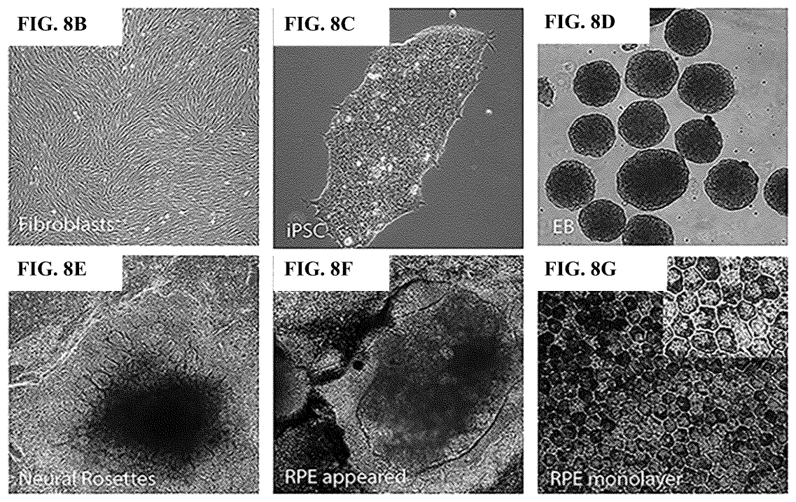

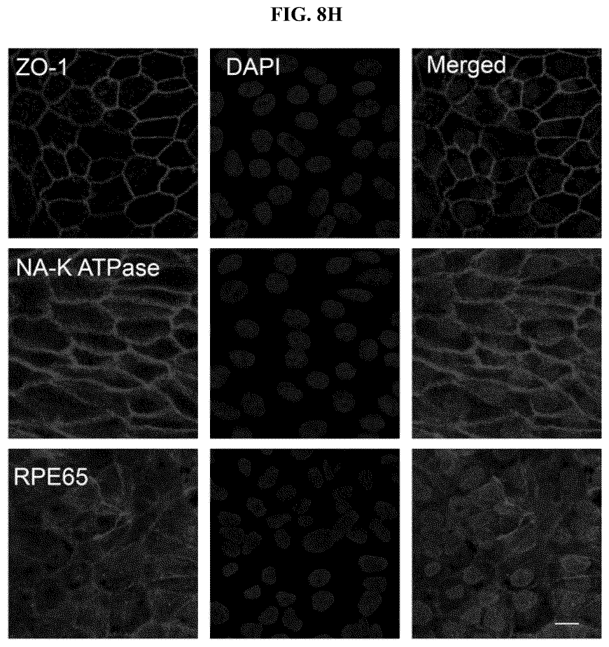

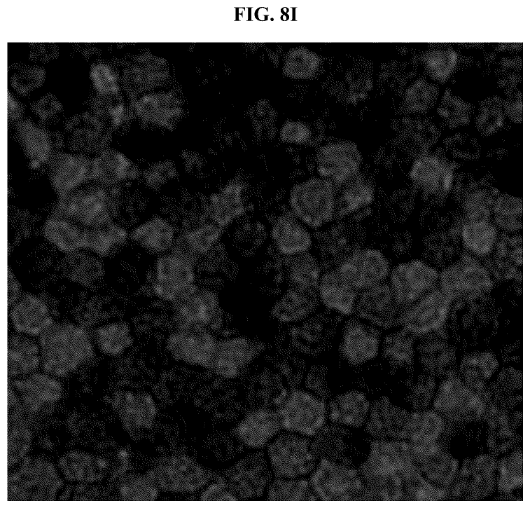

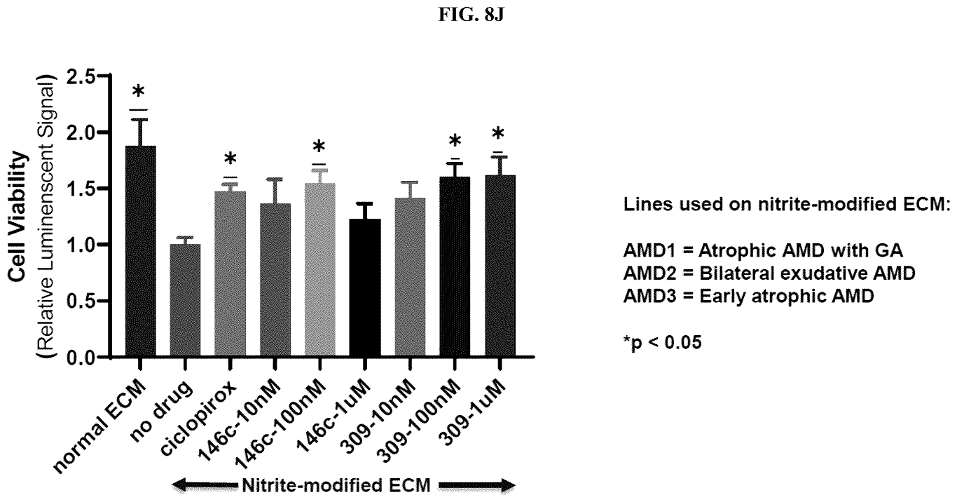

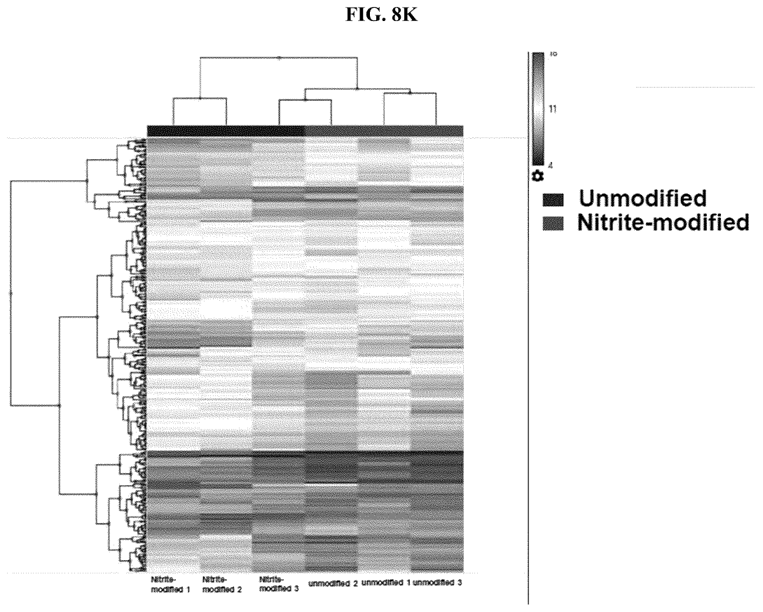

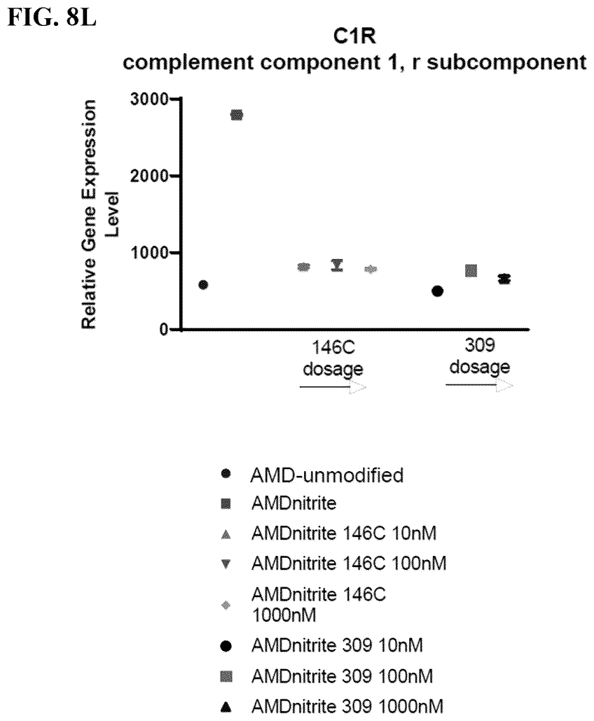

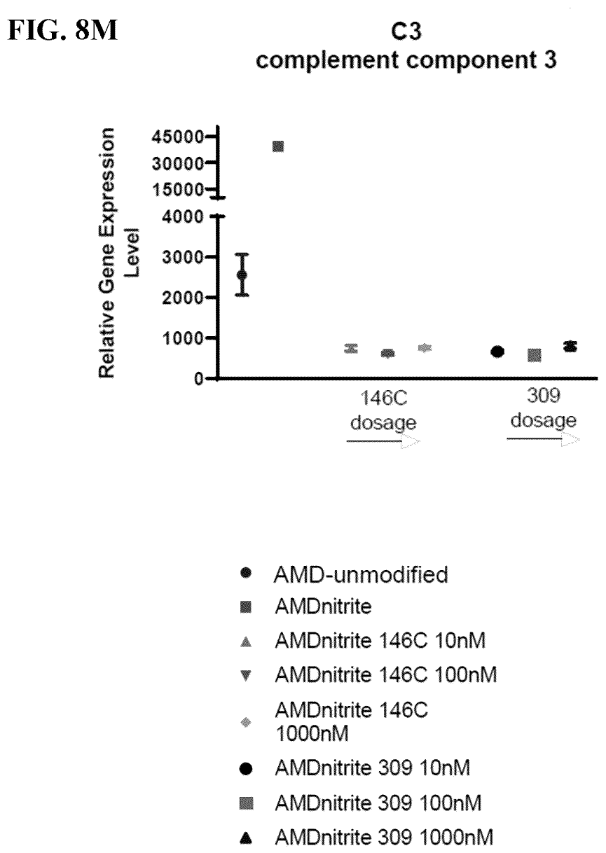

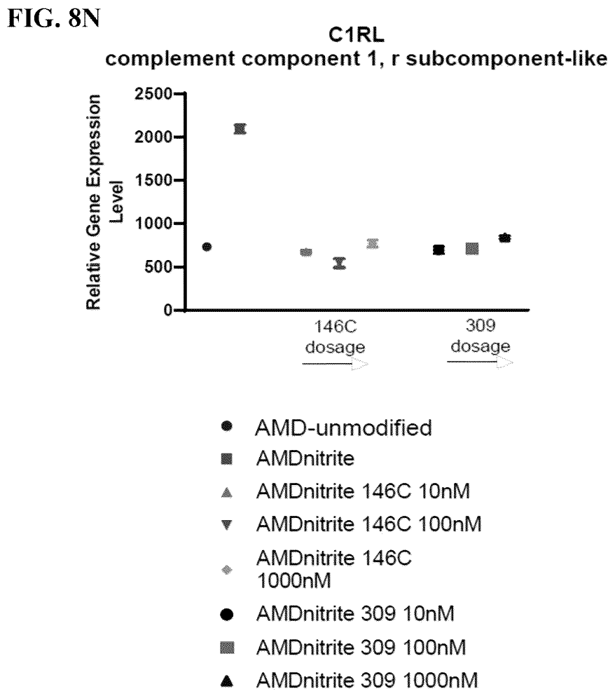

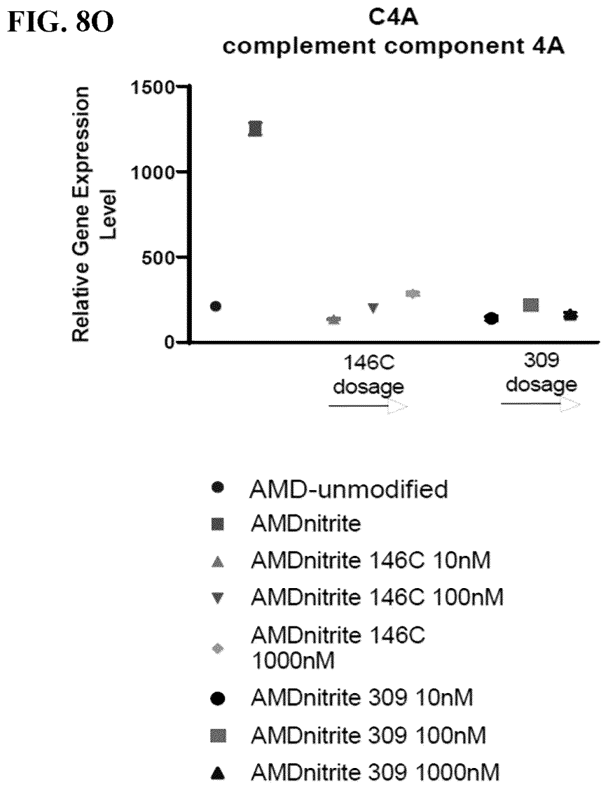

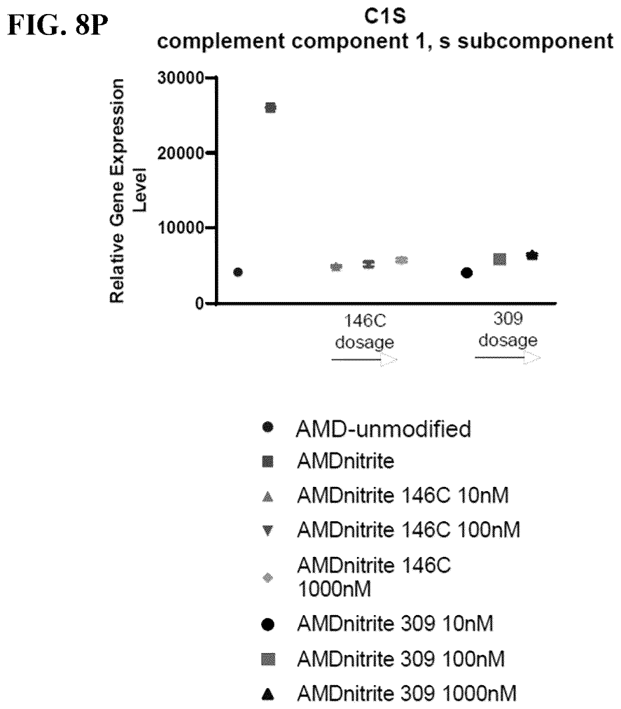

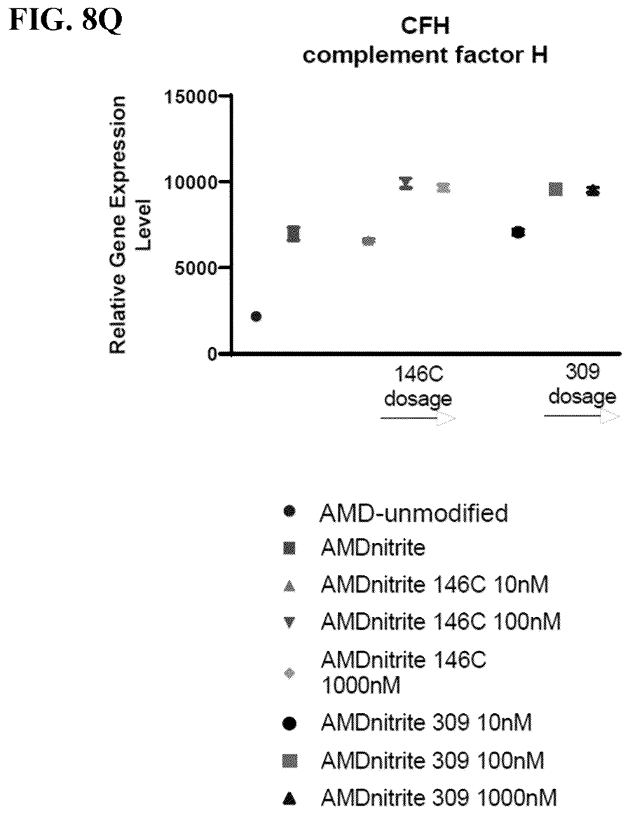

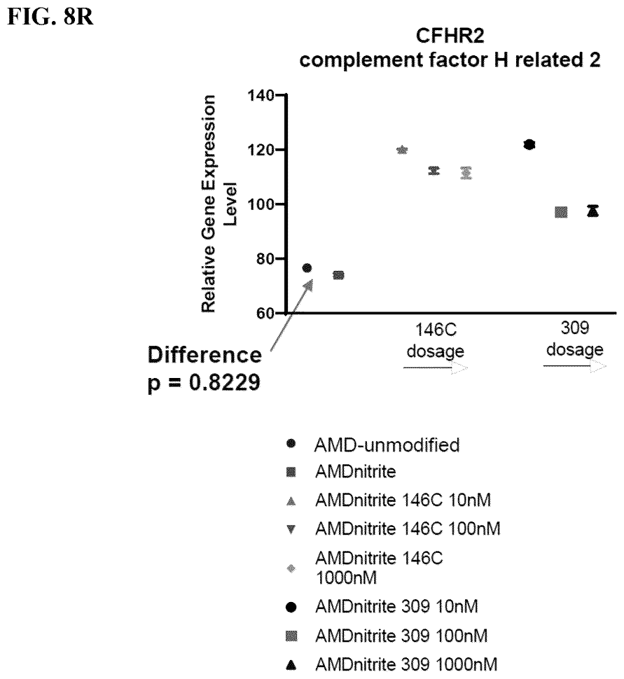

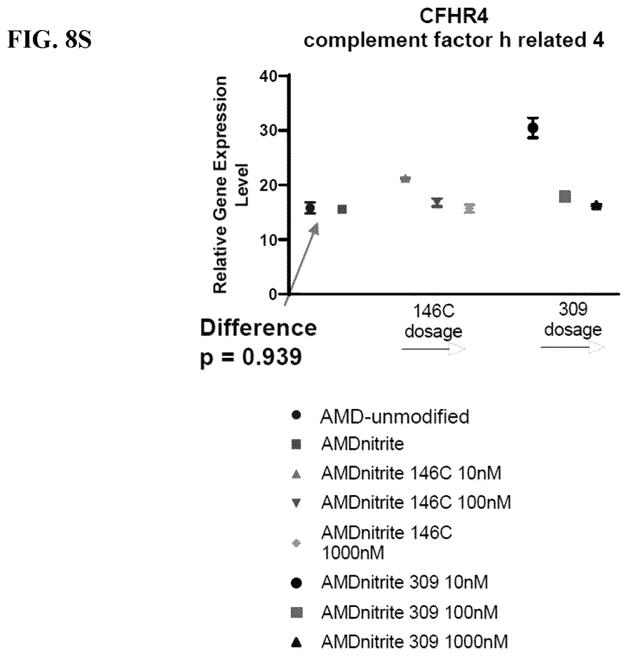

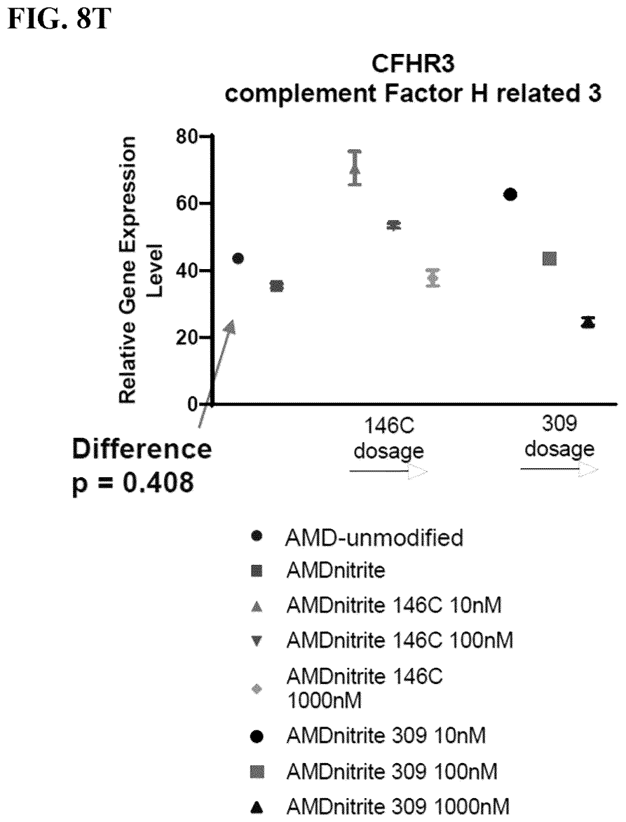

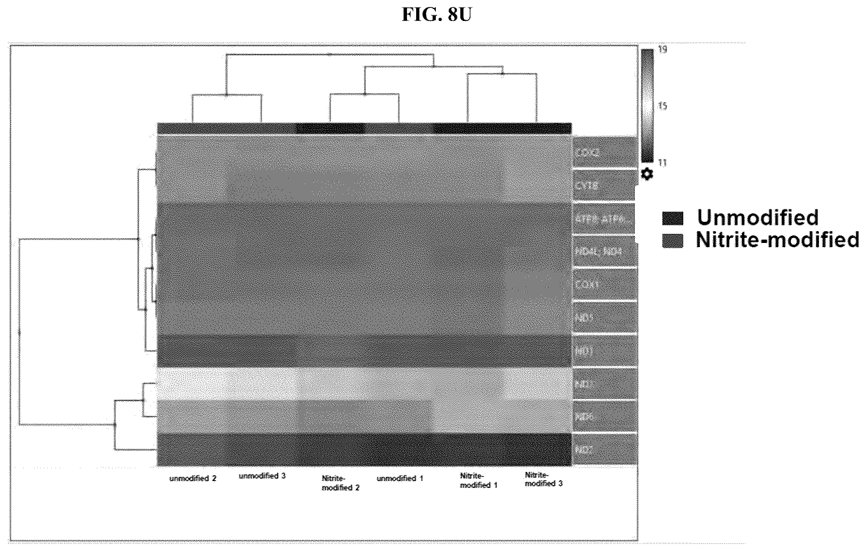

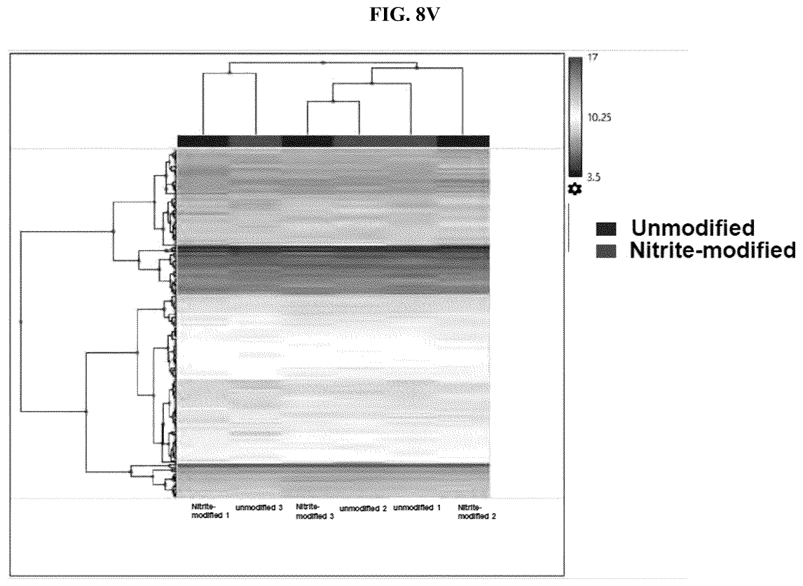

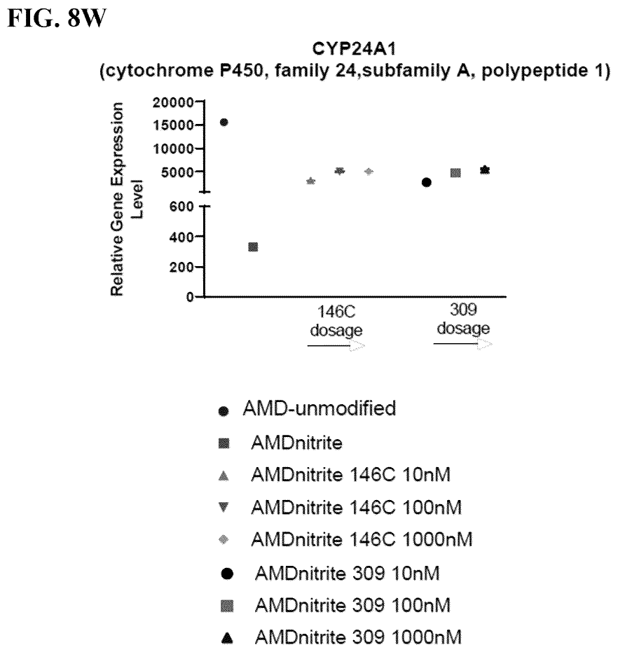

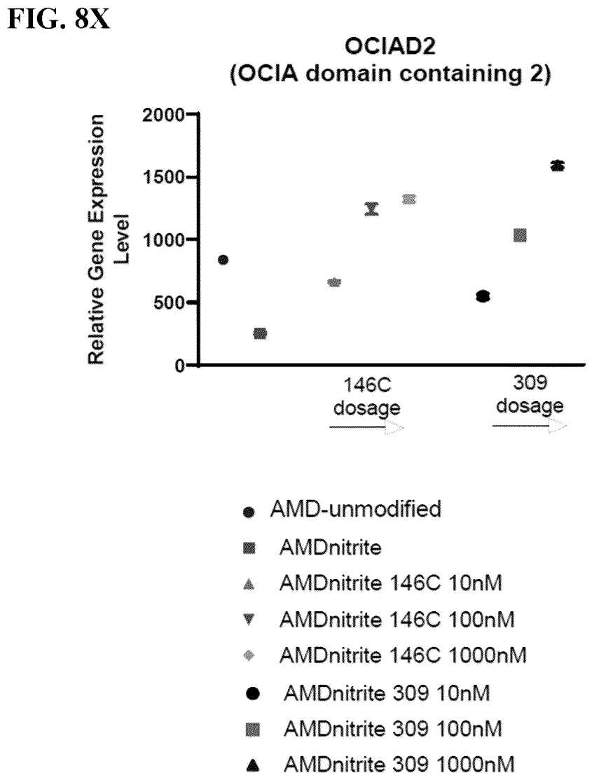

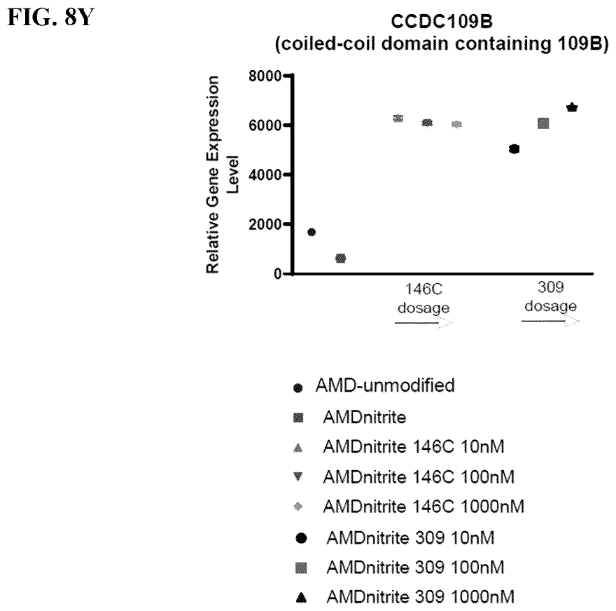

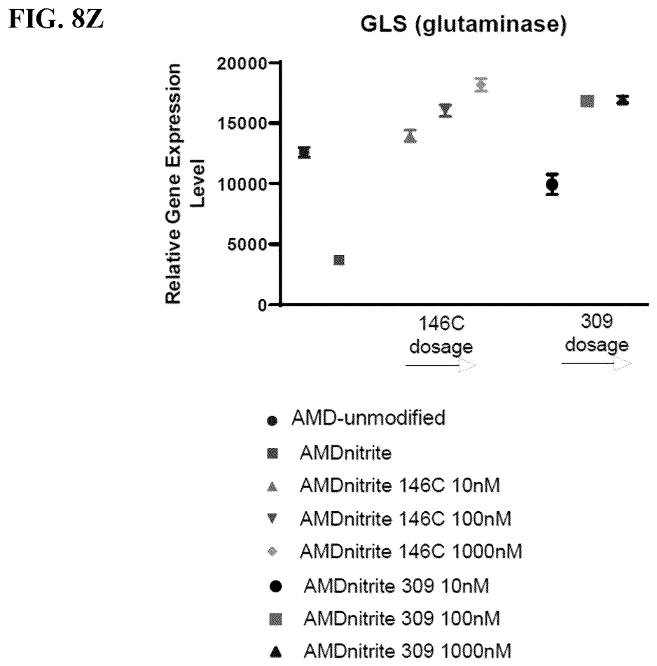

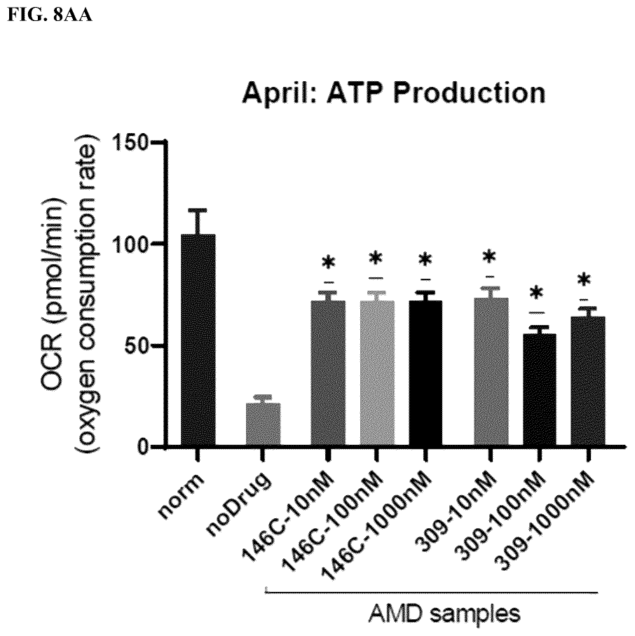

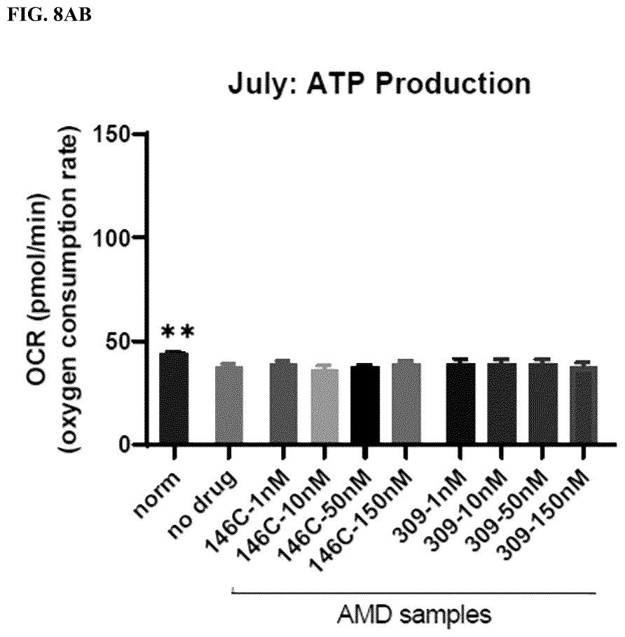

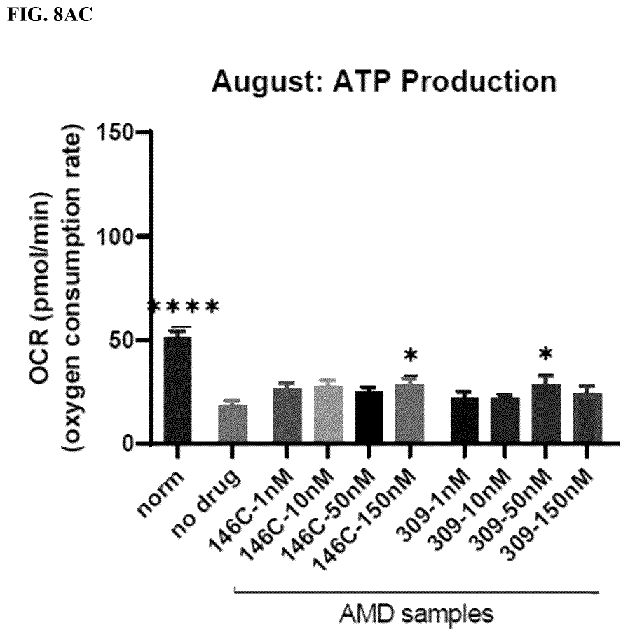

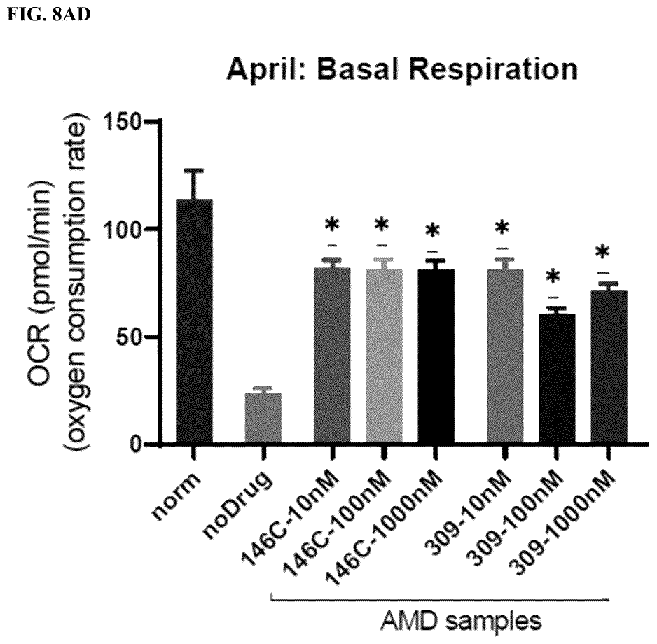

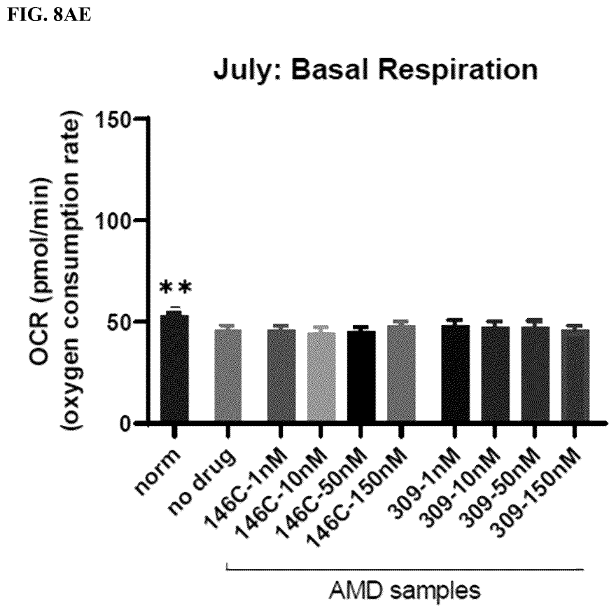

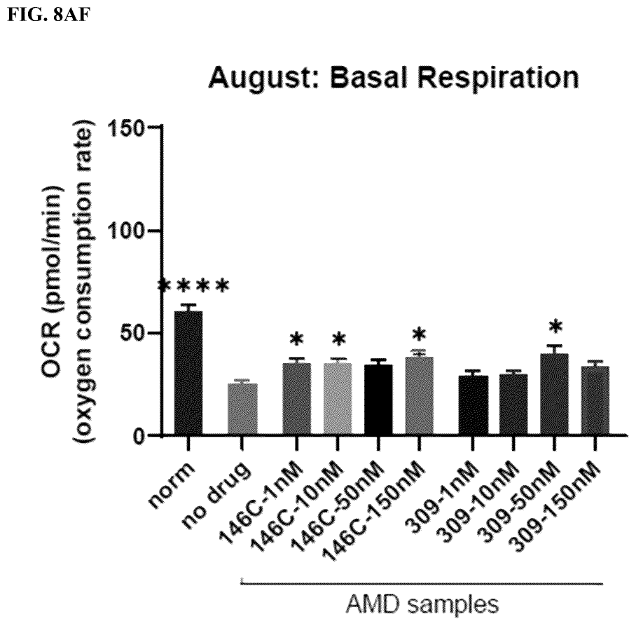

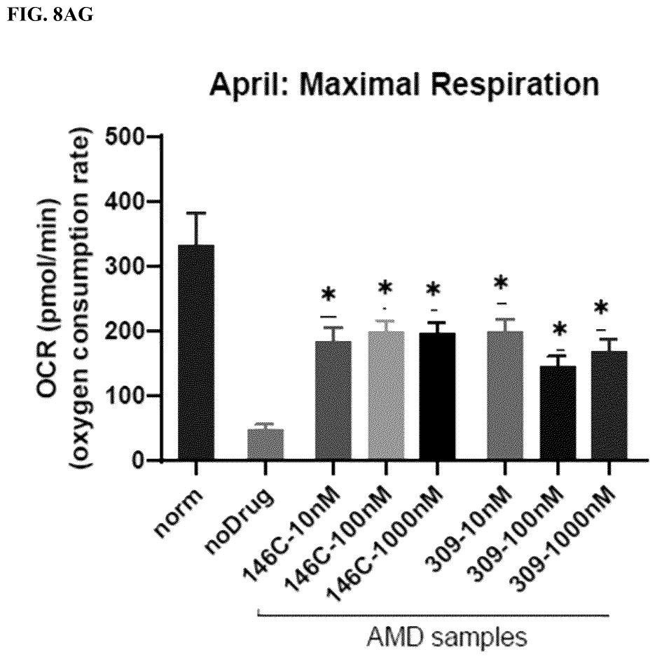

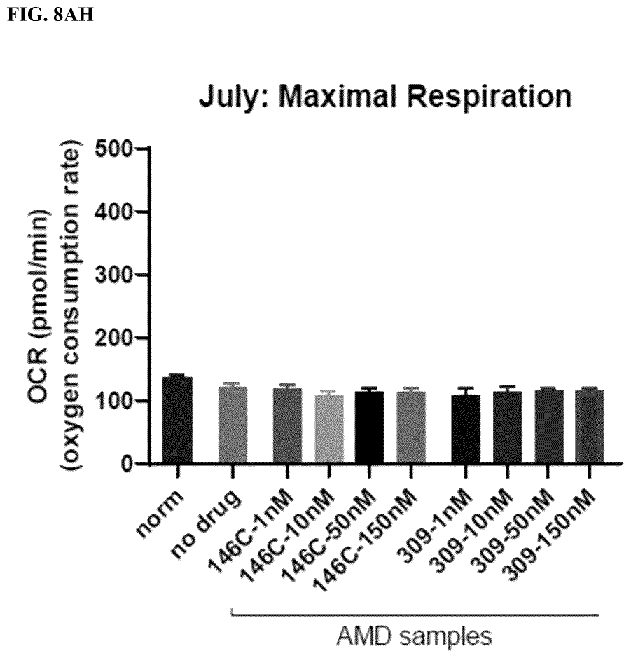

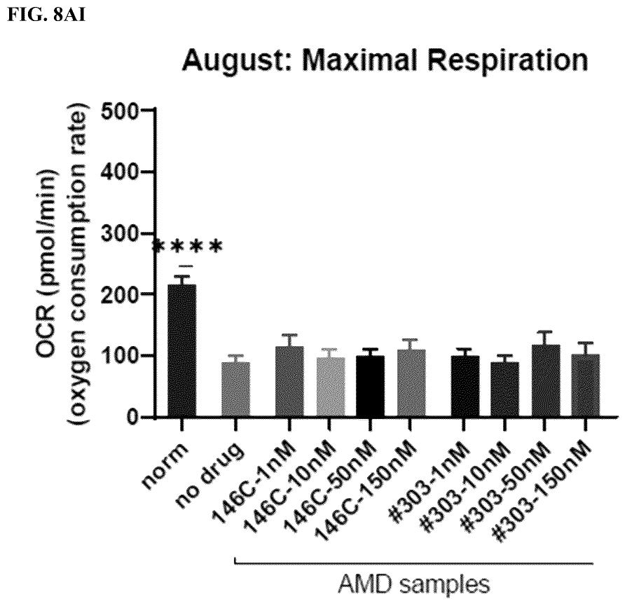

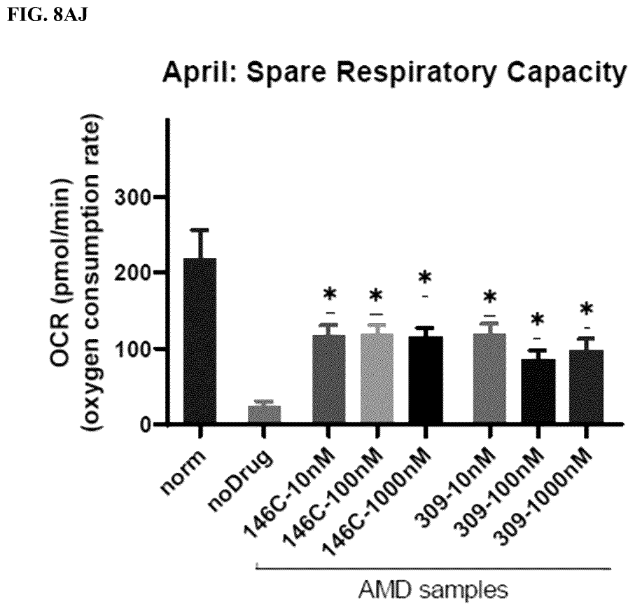

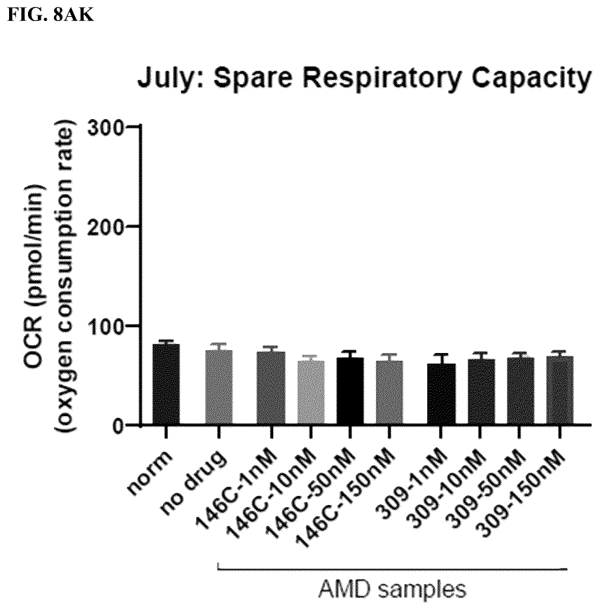

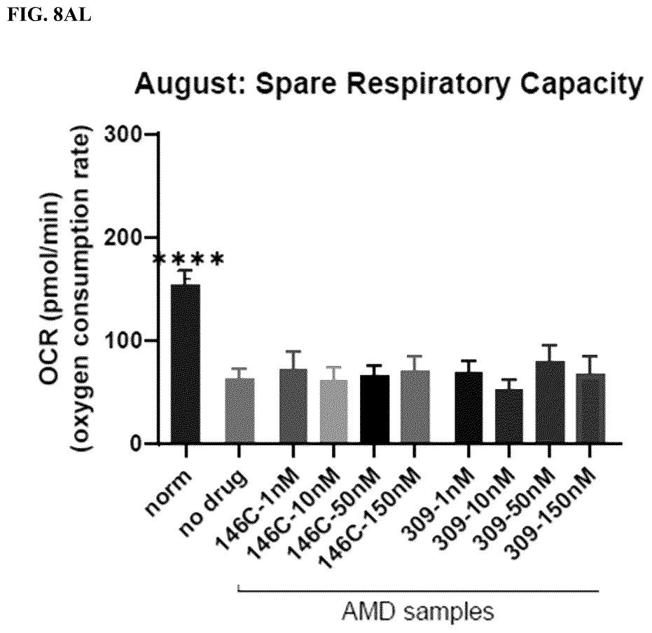

Cell viability assay. iPSC-derived RPE cells from AMD donors (n=3; 2 atrophic with GA, 1 exudative) were treated with drug (elamipretide, compound of Formula IIa, ciclopirox olamine, or vehicle) as described below and iPSC-derived RPE cells from non-diseased controls (n=2) were cultured on nontreated ECM or nitrite-treated ECM in vitro Bruch's membrane model for 24 hours. Only the cells from AMD donors received drug. The experimental approach is illustrated in FIG. 8A.

### Experimental Groups:

AMD donors only (n=3 individual donors)
   Cells treated with elamipretide (also referred to as “309” in the figures) at 10 nM, 100 nM and 1000 nM
   Cells treated with compound of Formula IIa (also referred to as “146c” in the figures) at 10 nM, 100 nM, 1000 nM
   Cells treated with positive control, ciclopirox olamine (also referred to as “ciclopirox” in the figures), at optimized dose
   Vehicle-treated cells
Non-diseased controls (n=2)

Cell viability was measured by Real Time-Glo MT Cell Viability Assay (Promega, Madison, WI, www.promega.com) according to the manufacturer's protocol. The assay measures the reducing potential of viable cells and is adenosine triphosphate (ATP)-independent. Luminescent signals were acquired using a BioTek FLx800 plate reader (BioTek).

Measurement of mitochondrial function. Analysis of mitochondrial function was performed on live iPSC-derived RPE cells from AMD donors (n=3; 2 atrophic with GA, 1 exudative) treated with drug (elamipretide, compound of Formula IIa, or vehicle) as described below and iPSC-derived RPE cells from non-diseased controls (n=2) using the XFe96 Extracellular Flux Analyzer (Agilent Technologies, Santa Clara, California, www.agilent.com) and the Seahorse XF Cell Mito Stress Test (CMST) Kit (Agilent Technologies). iPSC-derived RPE cells were seeded onto a laminin-coated Seahorse XF plates and grown until confluent. Data were normalized by cell count. Drug treatments began 24 hours prior to assay.

### Experimental Groups:

AMD donors only (n=3 individual donors)
   Cells treated with elamipretide (also referred to as “309” in the figures) at 10 nM, 100 nM and 1000 nM
   Cells treated with compound of Formula IIa (also referred to as “146c” in the figures) at 10 nM, 100 nM, 1000 nM
   Vehicle-treated cells
Non-diseased controls (n=2)

Cells were stained with DAPI and counted by ImageJ software (National Institute of Health, Bethesda, Maryland, www.nih.gov). Cells were then washed with CMST assay medium (XF base medium DMEM supplemented with 2 mM glutamine, 5.5 mM glucose, and 1 mM sodium pyruvate, pH 7.4; Agilent Technologies), followed by incubation for 1 hour at 37° C. in a non-CO₂ incubator. Oxygen consumption rate was detected under basal conditions followed by the sequential addition of oligomycin, carbonyl cyanide 4-(trifluoromethoxy) phenylhydrazone (FCCP), rotenone, and antimycin A. From these sequential additions, the following parameters can be derived: basal respiration, ATP production, maximal respiration, and spare respiratory capacity.

Microarray analysis. iPSC-derived RPE cells from AMD donors (n=3; 2 atrophic with GA, 1 exudative) were treated with drug (elamipretide or compound of Formula IIa) as described below and cultured on an in vitro Bruch's membrane model for 24 hours.

### Experimental Groups:

AMD donors only (n=3 individual donors)
   Cells treated with elamipretide (also referred to as “309” in the figures) at 10 nM, 100 nM and 1000 nM
   Cells treated with compound of Formula IIa (also referred to as “146c” in the figures) at 10 nM, 100 nM, 1000 nM

Microarray studies using Affymetrix GeneChip Human Clariom™ S Assay were performed as described in Gong et al. (2020).

Statistical analysis. Data, statistical analysis, and graphing were performed as described in Gong et al. (2020).

### Results:

Differentiation of human iPSCs into RPE cells. As shown in FIGS. 8B-8D, iPSCs from fibroblasts were induced to form embryoid bodies (EBs). Attached EBs then formed neural rosettes before RPE-like cells appeared in the culture (FIG. 8E). Hexagonal pigmented monolayer of RPE cells formed in culture (FIGS. 8F and 8G). These iPSC-derived PRE cells expressed RPE markers including the visual cycle protein retinal pigment epithelium-specific 65 kDa protein (RPE65), tight junction protein zonula occludens-1 (ZO-1), and sodium-potassium ATPase (NA-K ATPase) (FIG. 8H). FIG. 8I shows pigmented iPSC-derived RPE.

Cell viability on nitrite-modified ECM As shown in FIG. 8J, AMD-derived RPE exhibited reduced ability to survive on nitrite-modified ECM (“no drug” vehicle-treated cells), while both elamipretide and compound of Formula IIa significantly enhanced AMD-derived RPE cell viability on nitrite-modified ECM (model for a diseased Bruch's membrane).

Gene expression profile on nitrite-modified ECM. As shown in FIG. 8K, hierarchical cluster analysis (HCA) demonstrates that nitrite modification of the ECM induces clustering of the gene expression profile into two distinct groups.

The effects of elamipretide and compound of Formula IIa on complement related gene expression were examined. As shown in FIGS. 8L-8T, nitration of ECM increases expression of complement component genes, complement C1R (C1R), complement component 3 (C3), and complement C4A (C4A), among others. Both elamipretide and compound of Formula IIa reverse this trend (FIGS. 8L-8P). Both elamipretide and compound of Formula IIa increase expression of complement regulatory genes including complement factor H-related protein 2 (CFHR2) (FIG. 8S), a major complement regulator that inhibits the C3 alternative pathway. CFHR2 deficiency has been shown to correlate with systemic complement activation and increased risk of AMD. See, e.g., Zhang et al., BMC Med Genet 9:51 (2008); Kubista et al., Mol Vis 17:2080-2092 (2011); Eberhardt et al., PLoS One 8:e78617 (2013); and Cantsilieris et al., Proc Natl Acad Sci USA 115:E4433-4442 (2018).

Effects of elamipretide and compound of Formula IIa on mitochondrial-related gene expression. HCA does not appear to separate iPSC-derived RPE on unmodified versus nitrite-modified ECM for mitochondrial genes (FIG. 8U); however, there are significant changes in individual genes within this group (FIG. 8V). As shown in FIGS. 8W-8Z, both elamipretide and compound of Formula IIa altered the expression of mitochondrial-related genes, such as CYP24A1 (FIG. 8W) and GLS (glutaminase; FIG. 8Z). Single point variants in CYP24A1 (the gene encoding the catabolizing enzyme of the vitamin D pathway) have been shown to influence AMD. See Morrison et al., Hum Genomics 5(6):538-568 (2011).

Mitochondrial function. As shown in FIGS. 8AA-8AL, both elamipretide and compound of Formula IIa demonstrate efficacy in improving mitochondrial function (ATP production (FIGS. 8AA-8AC); basal respiration (FIGS. 8AD-8AF); maximal respiration (FIGS. 8AG-8AI); and spare respiratory capacity (FIGS. 8AJ-8AL) in AMD-derived RPE cells.

### Conclusion:

In summary, these results demonstrate that treatment with elamipretide and compound of Formula IIa significantly improve the ability of AMID-derived RPE cells to survive on nitrite-modified ECM, and treatment with elamipretide and compound of Formula IIa alter expression of mitochondrial and complement-related genes after nitration of ECM. Accordingly, these results demonstrate that elamipretide and compound of Formula IIa are useful in methods for treating, preventing, inhibiting, amelioration or delaying the onset of age-related macular degeneration, including dry AMD. Furthermore, given the results and the known penetration of the peptidomimetics e.g. of Formula IIa) in the parts of the eye as well as its propensity to target mitochondria, it is expected that these peptidomimetics will be useful in treating, preventing, inhibiting, ameliorating or delaying the onset of ophthalmic diseases, disorders and conditions generally, including without limitation, GA, glaucoma and/or wet or dry age-related macular degeneration. Furthermore, it is anticipated, based on these results, that the administration of the peptidomimetics will be useful in treating, preventing, inhibiting, ameliorating or delaying the onset of deterioration of the (mitochondria-rich) ellipsoid zone integrity in one or more eyes of a mammalian subject in need thereof.

### Example 3—Uptake of Compound of Formula IIa in Ocular Tissues in a Non-Human Primate Model

This example demonstrates that compound of Formula IIa is taken up by ocular tissues in concentrations typically suitable for producing a therapeutic effect when administered by subcutaneous injection for 28 days to cynomolgus monkeys.

### Summary

This study evaluated the uptake of the compound of Formula IIa in ocular tissues in cynomolgus monkeys when administered by subcutaneous (SC) injection once daily for 28 days.

Male and female cynomolgus monkeys were divided into four groups (Groups 1 to 4). Groups 1 and 4 consisted of five males and five females each and Groups 2 and 3 consisted of three males and three females each. Animals were dosed via SC injection once daily for 28 consecutive days. Group 1 animals received the control article, 0.9% sodium chloride injection, USP (saline). Group 2, 3, and 4 animals received the test article, compound of Formula IIa, at dose levels of 2, 5, and 15 mg/kg/day, respectively. Three males and three females from Groups 1 to 3, and three males and one female from Group 4 were necropsied on Day 29 (terminal necropsy). Two males and two females from Groups 1 and 4 were necropsied on Day 42 (recovery necropsy), after a 13-day treatment-free period. Dosing at 15 mg/kg/day (Group 4) was not well tolerated in two females who were necropsied early on Days 25 and 27. Safety endpoints included daily clinical and weekly detailed observations, food evaluation, dermal scoring of the injection site, body weight, ophthalmology, electrocardiography (ECG), hematology, coagulation, serum chemistry, and urinalysis. Blood was collected at multiple time points. At termination, gross observations and organ weights were recorded, and eye/optic nerve tissue samples were collected for microscopic evaluation and biodistribution assays.

### Methods

Test article. The test article was compound of Formula IIa.

Preparation of dose formulations. Dose formulation preparations were performed once weekly in a biosafety cabinet using clean techniques. The test article dosing formulations were prepared by diluting the 100 mg/mL (nominal concentration) compound of Formula IIa stock solution in the appropriate volume of 0.9% Sodium Chloride Injection, USP. All formulations (including Group 1, control) were filtered using a 0.22 μm polyethersulfone (PES) syringe filter. Osmolarity and pH were measured and recorded, then the dosing formulations were aliquoted into a sufficient number of sterile glass vials for daily use over one week. All aliquots were stored in a refrigerator set to maintain 4° C. and used within 7 days after preparation.

On the day of use, each container to be used was removed from 4° C. storage and transferred to the animal room for dosing. Dose administration was completed within 6 hours after removal from 4° C. storage. Any residual dosing formulation left in the “daily use” container after each day of was discarded.

Test system. The test system was cynomolgus monkey, originating from Cambodia, supplied by Worldwide Primates. The animals were identified by unique skin tattoos. The body weight of the animals ranged from 1.6. to 2.4 kg at initiation of dosing and the ages ranged from 2.0 to 3.1 years at the initiation of dosing. For acclimation, there were 18 males and 18 females. For dosing, 16 males and 16 females were used.

Animal welfare. The Testing Facility is accredited by the Association for Assessment and Accreditation of Laboratory Animal Care (AAALAC), has an Animal Welfare Assurance approved by the Office of Laboratory Animal Welfare (OLAW), is registered with the United States Department of Agriculture (USDA), and has an Institutional Animal Care and Use Committee (IACUC) responsible for the Testing Facility's compliance with applicable laws and regulations concerning the humane care and use of laboratory animals.

Housing and environmental conditions. Animals were housed in an environment controlled for temperature and humidity. The targeted range of temperature and relative humidity was between 18 and 29° C. and 30 and 70%, respectively. An automatic lighting system was set to provide a 12-hour light/dark cycle. The dark cycle was interrupted for study- or facility-related activities. The animals were socially-housed in cages that comply with the Animal Welfare Act and recommendations set forth in the Guide for the Care and Use of Laboratory Animals (National Research Council, 2011).

Diet and feeding. PMI's LabDiet® Fiber-Plus® Monkey Diet 5049 was provided at an appropriate daily ration. Animals were fasted prior to blood draws for serum chemistry, urine collection, or when procedures involving sedation or anesthesia were performed. The feed was routinely analyzed for contaminants and none were present at levels that interfered with the outcome of the study.

Drinking water. Fresh drinking water was provided ad libitum. The water was routinely analyzed for contaminants and none were present at levels that interfered with the outcome of the study.

Environmental enrichment. Fruits, vegetables, treats, as well as enrichment devices, were provided throughout the course of the study.

Veterinary treatments. None of the veterinary treatments such as treatment for diarrhea (pepto-bismol/lactobacillus/fiber and/or Tylosin) affected any animal as a test system for achieving the study objectives. Diphenhydramine or Diazepam was used for some animals, as necessary, when histaminergic or allergic response clinical observations were seen.

Experimental design. Animals were transferred to the study from a Testing Facility stock colony. Prior to transfer, selected animals were examined by veterinary staff to confirm suitable health condition. Animals were acclimated to the laboratory procedures over a minimum period of 14 days prior to initiation of dosing.

Randomization and animal assignment. For control of bias, animals were randomly assigned to groups based on established social unit and assigned study specific animal numbers.

Study experimental design. The experimental design is provided in Table 4.

TABLE 4
Study experimental design.

| | | Dose | | | | | | | |
|---|---|---|---|---|---|---|---|---|---|
| | | Level | Dose | Dose | Terminal | | Recovery | | |
| | Test | (mg/kg/ | Conc. | Volumeª | (Day 29) | | (Day 42) | | Early |
| Group | Material | day) | (mg/mL) | (mL/kg) | M | F | M | F | Necropsy |
| 1 | Control | 0 | 0 | 0.15 | 1001 | 1501 | 1004, | 1504, | NA |
| | | | | | to | to | 1005 | 1505 | |
| | | | | | 1003 | 1503 | | | |
| 2 | Cmpd. of | 2 | 13.3 | 0.15 | 2001 | 2501 | NA | NA | NA |
| | Formula | | | | to | to | | | |
| | IIa | | | | 2003 | 2503 | | | |
| 3 | Cmpd. of | 5 | 33.3 | 0.15 | 3001 | 3501 | NA | NA | NA |
| | Formula | | | | to | to | | | |
| | IIa | | | | 3003 | 3503 | | | |
| 4 | Cmpd. of | 15 | 100 | 0.15 | 4001 | 4503 | 4004, | 4504, | 4501 |
| | Formula | | | | to | | 4005 | 4505 | (F, Day |
| | IIa | | | | 4003 | | | | 25) |
| | | | | | | | | | 4502 |
| | | | | | | | | | (F, Day |
| | | | | | | | | | 27) |

Conc. = Concentration;
F = Female;
M = Male;
NA = Not applicable
^{a}Individual dose volumes (mL) were calculated based on the most recent body weight.

Administration of dose formulations. The dose formulations were administered to appropriate animals by subcutaneous injection into the interscapular area once daily for 28 days using disposable syringe and needle. Four subcutaneous dose sites (upper left/right and lower left/right) were designated on the dorsal back using permanent marker or tattoo placed during acclimation avoiding the spine, with no edges overlapping and with a maximum practical distance between the dose sites. The designated dosing sites were shaved, and injection was rotated among them. If a designated site was not suitable for administration (e.g., due to wound, scab/crust, etc.), the next suitable site was used for dosing and appropriately documented.

The SC administration route of exposure was consistent with the proposed route of administration in humans.

Clinical observations. A mortality check was conducted twice daily to assess general animal health and wellness (except on the first and last day of the in-life phase where it was performed at least once). Cage side clinical observations were performed once daily, beginning on the second day of acclimation. On dosing days, the clinical observations were conducted 2 hours (±0.5 hours) post-dose. A detailed clinical examination was performed on Day −1, and weekly thereafter throughout the in-life phase (Days 7, 14, 21, 28, 29, 35, and 42, prior to dose on dosing days). Animals were placed in a procedure cage for the examination.

Body weight. Body weights were measured twice during acclimation (including Day −1), then weekly during the in-life phase (Days 7, 14, 21, 28, 35, and 41) and on the day of corresponding necropsy (Day 29 or Day 42).

Ophthalmology. Ophthalmology examinations were performed once during acclimation (Day −12) and on Day 24 (Week 4). Topical mydriatic was administered. Examination during the recovery period was not conducted since there were no compound of Formula IIa-related findings at Week 4.

Necropsy. Animals were fasted overnight prior to termination. Following blood collection on the day of necropsy, animals were sedated, weighed, and euthanized by an overdose of euthanasia solution, followed by whole body perfusion flush with phosphate buffered saline. Animals were subjected to a complete macroscopic examination and tissue collection. Bone marrow smears were prepared from the sternum at scheduled necropsy. Two females were necropsied early on Days 25 and 27. Blood samples (hematology, coagulation, and serum chemistry) were collected prior to euthanasia, after which a complete macroscopic examination was performed.

Tissue collection and preservation. Left eyes and optic nerves were fixed in a solution of 2.5% NBF and 3% Glutaraldehyde, and right eyes and optic nerves were frozen for biodistribution.

Biodistribution analysis—right eye and optic nerve. Following flash freezing in liquid nitrogen for 10-20 seconds, the specimens were placed on dry ice, then stored in a freezer set to maintain −80° C. All specimens were shipped frozen on dry ice via overnight courier to the testing facility for compound of Formula IIa biodistribution analysis in the eye and optic nerve.

A total of 32 cynomolgus monkey eyes were received at the study test site. The eyes were stored at −80° C. until tissue harvesting to collect aqueous humor (AH), vitreous human (VH), conjunctiva, cornea, iris/ciliary body (ICB), lens, retina, choroid, optic nerve, and sclera. The method utilized protein precipitation (PPT) followed by instrumental analysis using HPLC-MS/MS.

Tissue homogenization. To homogenize ocular tissue samples, weighed amounts of control bovine conjunctiva, ICB, lens, cornea, retina, sclera, choroid, and optic nerve (provided by PharmOptima, Portage, Michigan) were homogenized in USA scientific impact resistant microtubes containing 2.8 mm ceramic beads. Unknown cynomolgus monkey conjunctiva, ICB, lens, cornea, retina, sclera, choroid, and optic nerve samples were homogenized in USA scientific impact resistant microtubes containing 2.8 mm ceramic beads. Using a diluent of water: acetonitrile: formic acid (75:25:0.1, v/v/v), cornea and conjunctiva samples were diluted 1:19 (parts tissue to parts diluent), retina samples were diluted 1:4, sclera, lens, and optic nerve samples were diluted 1:9, choroid and ICB samples were diluted 1:14. Tissues were homogenized (Precellys® Evolution temperature at 4° C.) at 5500 rpm for 3×30 second cycles with 20 second pauses between cycles until homogenized. Conjunctiva, cornea, choroid, and sclera samples went through four runs of homogenization, and ICB, lens, retina and optic nerve samples went through one run of homogenization.

Calibration standards. Stock standards were prepared by individually diluting a weighed amount of compound of Formula IIa with water: formic acid (1000:1, v/v), to result in a final concentration of 1000 μg/mL. A working stock was prepared by individually diluting 40 μL of 1000 μg/mL stock standard with 360 μL of water: formic acid (1000:1, v/v) for a final concentration of 100 μg/mL of compound of Formula IIa. Working calibration standards of compound of Formula IIa were prepared by serially diluting the working stock standard over a range of 10.0 ng/mL to 20,000 ng/mL. Working calibration standards of compound of Formula IIa were prepared for lens samples by serially diluting the working stock standard over a range of 50.0 ng/mL to 100,000 ng/mL.

Quality controls. Stock standards were prepared by individually diluting a weighed amount of compound of Formula IIa with water: formic acid (1000:1, v/v), to result in a final concentration of 1000 μg/mL. A working QC stock was prepared by diluting 20.0 μL of the 1000 μg/mL stock with 180 μL of water: formic acid (1000:1 v/v) for a final concentration of 100 μg/mL of compound of Formula IIa. QC samples were prepared by serially diluting the working QC stock for concentrations in matrix for Low, Mid, and High QC levels of 6.00, 100, and 1,600 ng/mL. Vitreous humor QCs were prepared by serially diluting the working QC stocks for concentrations in matrix for Low, Mid, and High QC levels of 12.0, 200, and 3,200 ng/mL. Optic nerve QCs were prepared by serially diluting the QC stock for concentrations in matrix for Low, Mid, and High QC levels of 15.0, 500, and 8,000 ng/mL.

Blanks, Blanks with IS, Unknowns and Extraction Procedure for Aqueous Humor, Vitreous Humor, and Tissue Matrices. In a 2 mL 96-well plate, 100 μL of unknown vitreous humor, aqueous humor, or unknown tissue homogenate, QC, standard, or blank control matrix homogenate) was added. Twenty (20) μL of WIS (5000 ng/mL Formula IIb in water: acetonitrile [1:1 v/v]) was added to blanks or 20 μL of acetonitrile: water (1:1 v/v) was added to double blanks (blanks without internal standard). Two hundred (200) μL of acetonitrile) was added to all samples. The compound of Formula IIb is a deuterated version of the compound of Formula IIa. This compound was prepared by substitution of a deuterated L-lysine for standard L-lysine in the preparation used to make the compound of Formula II, and salts thereof.

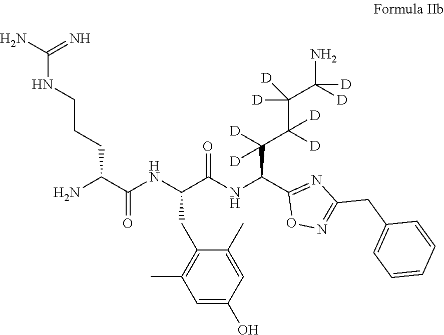 [SMILES: [2H]C([2H])(N)C([2H])([2H])C([2H])([2H])C([2H])([2H])[C@H](NC(=O)[C@H](Cc1c(C)cc(O)cc1C)NC(=O)[C@H](N)CCCNC(=N)N)c1nc(Cc2ccccc2)no1]

Each D is deuterium substituted for hydrogen

For optic nerve samples, 50.0 μL of unknown tissue homogenate, QC, standard, or blank control matrix homogenate) was added. Twenty (20) μL of WIS (5000 ng/mL Formula IIb in water: acetonitrile [1:1 v/v]) was added to blanks or 20 μL of acetonitrile: water (50:50 v/v) was added to double blanks (blanks without internal standard). Two hundred (200) μL of acetonitrile was added to all samples.

Samples were vortex mixed for 5 minutes and centrifuged at 4000 rpm (4° C.) for 10 minutes. One hundred (100) μL of water: formic acid (1000:2 v/v) was added to 100 μL supernatant in a 96-well collection plate and mixed with a multichannel pipette and analyzed by LC-MS/MS.

Optic nerve samples were vortex mixed for 5 minutes and centrifuged at 4000 rpm (4° C.) for 10 minutes. Two hundred (200) μL of water: formic acid (1000:2 v/v) was added to 50.0 μL supernatant in a 96-well collection plate and mixed with a multichannel pipette and analyzed by LC-MS/MS.

### MS Conditions:

| | |
|---|---|
| Instrument: | Thermo Quantum Ultra LC-MS/MS |
| Data collection system: | LC Quan, version 2.5.6 |
| Polarity: | Positive |
| MS Mode: | Selective Reaction Monitoring (SRM) |
| Scan Time: | 300 msec/transition |
| Ionization: | Heated Electrospray (HESI) |
| Transition: | 608.5 ⇒ 303.3 (CE 33 V) for compound of |
| | Formula IIa |
| | 608.5 ⇒ 591.4 (CE 28 V) for compound of |
| | Formula IIa |
| | 616.3 ⇒ 303.3 (CE 35 V) for Formula IIb |
| Spray Voltage: | 4500 V |
| Vaporizer Temperature: | 300° C. |
| Sheath Gas Pressure: | 10 |
| Auxiliary Gas Pressure: | 40 |
| Capillary Temperature: | 270° C. |

### HPLC Conditions:

| | |
|---|---|
| Injection Volume: | 10 μL |
| Flow Rate: | 0.400 mL/minute |
| Mobile Phases: | A: Water:Formic acid (1000:1, v/v) |
| | B: Acetonitrile: Formic acid (1000:1, v/v) |
| \grave{ }Gradient: | 0.0-0.2 minutes: isocratic 0% B |
| | 0.2-2.5 minutes: linear gradient 30% B |
| | 2.5-2.6 minutes: linear gradient 90% B |
| | 2.6-4.4 minutes: : isocratic 90% B |
| | 4.4-4.5 minutes: linear gradient 0% B |
| | 4.5-5.5 minutes: re-equilibrate 0% B |
| Column: | Thermo Hypersil Gold, 50 × 2.1 mm, 1.9 micron |
| Needle Wash 1: | Acetonitrile:Formic acid (1000:1, v/v) |
| Needle Wash 2: | Acetonitrile:Water:Formic acid (50:50:0.1, v/v/v) |

Calculations. Percent coefficient of variation was used as an estimate of precision. Percent Coefficient of Variation (% CV)=(Standard Deviation/average value)*100. Quadratic least squares analysis: The standard curve fit was determined using a quadratic equation with 1/x² weighting: y=ax²+bx+c, where y=peak area ratio of the calibration standards to internal standard; x=concentration of the calibration standard; a=quadratic coefficient of x²; b=quadratic coefficient of x; and c=the constant as the y-intercept of the calibration curve. Quadratic analyte concentration: The concentration of analyte was calculated using the calibration curve parameters calculated above and then solving for the value of x.

### Results

Concentrations of drug in ocular matrices. The average concentrations of the compound of Formula IIa in various ocular tissues are presented in Table 5. Individual concentration results for the compound of Formula IIa ocular matrices are included in Tables 6-15.

TABLE 5
Average Concentrations of Compound of Formula IIa in Cynomolgus Monkey Ocular
Tissues following SC Administration.

| | | Average Concentration (ng/mL, AH and VH) (ng/g, all others) | | | | | |
|---|---|---|---|---|---|---|---|
| | | Day/Dose Route/Dose (mg/kg) | | | | | |
| | | Day 29 | Day 29 | Day 29 | Day 29 | Day 42 | Day 42 |
| Matrix | Gender | SC/0 | SC/2 | SC/5 | SC/15 | SC/0 | SC/15 |
| Sclera | Male | <LLOQ | 701 | 1820 | 2260 | <LLOQ | 3870 |
| | Female | <LLOQ | 446 | 837 | 7850 | <LLOQ | 4700 |
| Conjunctiva | Male | 10.9 | 603 | 3560 | 5430 | 9.75 | 4380 |
| | Female | <LLOQ | 835 | 1770 | 18900 | <LLOQ | 4540 |
| Cornea | Male | <LLOQ | 39.3 | 64.0 | 195 | <LLOQ | 34.8 |
| | Female | <LLOQ | 27.2 | 56.1 | 3110 | <LLOQ | 149 |
| Lens | Male | <LLOQ | <LLOQ | <LLOQ | <LLOQ | <LLOQ | <LLOQ |
| | Female | <LLOQ | <LLOQ | <LLOQ | 10.7 | <LLOQ | <LLOQ |
| ICB | Male | <LLOQ | 354 | 1230 | 3910 | <LLOQ | 1410 |
| | Female | <LLOQ | 375 | 800 | 6870 | <LLOQ | 3160 |
| Aqueous | Male | <LLOQ | 2.31 | 5.29 | 14.5 | <LLOQ | 3.82 |
| Humor | Female | <LLOQ | 2.76 | 25.9 | 133 | <LLOQ | 9.83 |
| Vitreous | Male | <LLOQ | 11.2 | 13.3 | 48.8 | <LLOQ | 45.3 |
| Humor | Female | <LLOQ | 13.1 | 23.3 | 34.6 | <LLOQ | 62.7 |
| Retina | Male | <LLOQ | 2770 | 5760 | 26100 | <LLOQ | 30200 |
| | Female | <LLOQ | 7390 | 20100 | 38900 | <LLOQ | 67000 |
| Choroid | Male | <LLOQ | 1890 | 4060 | 8790 | <LLOQ | 7880 |
| | Female | <LLOQ | 2500 | 3210 | 13000 | <LLOQ | 27600 |
| Optic Nerve | Male | <LLOQ | 22000 | 26300 | 60000 | <LLOQ | 91500 |
| | Female | <LLOQ | 17500 | 31800 | 63100 | <LLOQ | 40800 |

Units: ng/mL, AH and VH; ng/g, Sclera, Conjunctiva, Cornea, Lens, ICB, Retina, Choroid, and Optic Nerve
Day 29—Terminal necropsy
Day 42—Recovery necropsy
LLOQ = Lower Limit of Quantitation

TABLE 6
Concentration of Compound of Formula IIa in Cynomolgus Monkey
Aqueous Humor following SC Administration.

| | | | | | Average | | |
|---|---|---|---|---|---|---|---|
| Animal | | | | Concentration | Concentration | Std. | |
| ID | Gender | Eye | Dose Route | (ng/mL) | (ng/mL) | Dev | % CV |
| 1001 | Male | OD | Subcutaneous 0 | <LLOQ | <LLOQ | ISD | ISD |
| 1002 | | OD | mg/kg | <LLOQ | | | |
| 1003 | | OD | | <LLOQ | | | |
| 1501 | Female | OD | | <LLOQ | <LLOQ | ISD | ISD |
| 1502 | | OD | | <LLOQ | | | |
| 1503 | | OD | | <LLOQ | | | |
| 2001 | Male | OD | Subcutaneous 2 | 4.00 | 2.31 | 2.07 | 89.6 |
| 2002 | | OD | mg/kg | <LLOQ | | | |
| 2003 | | OD | | 2.94 | | | |
| 2501 | Female | OD | | 2.92 | 2.76 | 0.936 | 33.9 |
| 2502 | | OD | | 1.75 | | | |
| 2503 | | OD | | 3.60 | | | |
| 3001 | Male | OD | Subcutaneous 5 | 5.05 | 5.29 | 3.90 | 73.7 |
| 3002 | | OD | mg/kg | 1.51 | | | |
| 3003 | | OD | | 9.30 | | | |
| 3501 | Female | OD | | 1.34 | 25.9 | 24.9 | 96.1 |
| 3502 | | OD | | 51.1 | | | |
| 3503 | | OD | | 25.4 | | | |
| 4001 | Male | OD | Subcutaneous 15 | 19.9 | 14.5 | 10.9 | 75.2 |
| 4002 | | OD | mg/kg | 1.99 | | | |
| 4003 | | OD | | 21.7 | | | |
| 4501 | Female | OD | | 293 | 133 | 142 | 107 |
| 4502 | | OD | | 86.0 | | | |
| 4503 | | OD | | 19.9 | | | |
| 1004 | Male | OD | Subcutaneous 0 | <LLOQ | <LLOQ | ISD | ISD |
| 1005 | | OD | mg/kg | <LLOQ | | | |
| 1504 | Female | OD | | <LLOQ | <LLOQ | ISD | ISD |
| 1505 | | OD | | <LLOQ | | | |
| 4004 | Male | OD | Subcutaneous 15 | 2.32 | 3.82 | ISD | ISD |
| 4005 | | OD | mg/kg | 5.32 | | | |
| 4504 | Female | OD | | 3.76 | 9.83 | ISD | ISD |
| 4505 | | OD | | 15.9 | | | |

LLOQ = 1.00 ng/ml
ISD—Insufficient data for determination

TABLE 7
Concentration of Compound of Formula IIa in Cynomolgus Monkey Vitreous
Humor following SC Administration.

| | | | | | Average | | |
|---|---|---|---|---|---|---|---|
| Animal | | | | Concentration | Concentration | Std. | |
| ID | Gender | Eye | Dose Route | (ng/mL) | (ng/mL) | Dev | % CV |
| 1001 | Male | OD | Subcutaneous 0 | <LLOQ | <LLOQ | ISD | ISD |
| 1002 | | OD | mg/kg | <LLOQ | | | |
| 1003 | | OD | | <LLOQ | | | |
| 1501 | Female | OD | | <LLOQ | <LLOQ | ISD | ISD |
| 1502 | | OD | | <LLOQ | | | |
| 1503 | | OD | | <LLOQ | | | |
| 2001 | Male | OD | Subcutaneous 2 | 3.95 | 11.2 | 12.7 | 113 |
| 2002 | | OD | mg/kg | 3.81 | | | |
| 2003 | | OD | | 25.8 | | | |
| 2501 | Female | OD | | 6.59 | 13.1 | 8.56 | 65.3 |
| 2502 | | OD | | 9.94 | | | |
| 2503 | | OD | | 22.8 | | | |
| 3001 | Male | OD | Subcutaneous 5 | 20.6 | 13.3 | 9.78 | 73.5 |
| 3002 | | OD | mg/kg | 2.22 | | | |
| 3003 | | OD | | 17.2 | | | |
| 3501 | Female | OD | | 9.35 | 23.3 | 24.8 | 106 |
| 3502 | | OD | | 52.0 | | | |
| 3503 | | OD | | 8.62 | | | |
| 4001 | Male | OD | Subcutaneous 15 | 18.3 | 48.8 | 26.6 | 54.5 |
| 4002 | | OD | mg/kg | 61.0 | | | |
| 4003 | | OD | | 67.0 | | | |
| 4501 | Female | OD | | 44.5 | 34.6 | 13.1 | 37.9 |
| 4502 | | OD | | 19.8 | | | |
| 4503 | | OD | | 39.6 | | | |
| 1004 | Male | OD | Subcutaneous 0 | <LLOQ | <LLOQ | ISD | ISD |
| 1005 | | OD | mg/kg | <LLOQ | | | |
| 1504 | Female | OD | | <LLOQ | <LLOQ | ISD | ISD |
| 1505 | | OD | | <LLOQ | | | |
| 4004 | Male | OD | Subcutaneous 15 | 44.1 | 45.3 | ISD | ISD |
| 4005 | | OD | mg/kg | 46.5 | | | |
| 4504 | Female | OD | | 109 | 62.7 | ISD | ISD |
| 4505 | | OD | | 16.3 | | | |

LLOQ = 1.00 ng/ml
ISD—Insufficient data for determination

TABLE 8
Concentration of Compound of Formula IIa in Cynomolgus Monkey Conjunctiva following SC Administration.

| | | | | | Total | | | Average | | |
|---|---|---|---|---|---|---|---|---|---|---|
| Animal | | | | Tissue | Volume | Concentration | Concentration | Concentration | Std. | |
| ID | Gender | Eye | Dose Route | Weight (g) | (μL) | (ng/mL) | (ng/g) | (ng/g) | Dev | % CV |
| 1001 | Male | OD | Subcutaneous | 0.01584 | 317 | <LLOQ | <LLOQ | 10.9 | ISD | ISD |
| 1002 | | OD | 0 mg/kg | 0.06110 | 1222 | 1.63* | 32.6 | | | |
| 1003 | | OD | | 0.11808 | 1118 | <LLOQ | <LLOQ | | | |
| 1501 | Female | OD | | 0.02026 | 405 | <LLOQ | <LLOQ | <LLOQ | ISD | ISD |
| 1502 | | OD | | 0.08049 | 1080 | <LLOQ | <LLOQ | | | |
| 1503 | | OD | | 0.03844 | 768 | <LLOQ | <LLOQ | | | |
| 2001 | Male | OD | Subcutaneous | 0.01940 | 388 | 42.3 | 846 | 603 | 381 | 63.2 |
| 2002 | | OD | 2 mg/kg | 0.00985 | 197 | 8.22 | 164 | | | |
| 2003 | | OD | | 0.04771 | 954 | 39.9 | 798 | | | |
| 2501 | Female | OD | | 0.01487 | 298 | 14.4 | 289 | 835 | 705 | 84.4 |
| 2502 | | OD | | 0.02299 | 460 | 81.3 | 1630 | | | |
| 2503 | | OD | | 0.07795 | 1078 | 42.3 | 585 | | | |
| 3001 | Male | OD | Subcutaneous | 0.02870 | 574 | 287 | 5740 | 3560 | 1910 | 53.7 |
| 3002 | | OD | 5 mg/kg | 0.09456 | 1095 | 189 | 2190 | | | |
| 3003 | | OD | | 0.01853 | 371 | 138 | 2760 | | | |
| 3501 | Female | OD | | 0.11489 | 1115 | 184 | 1790 | 1770 | 932 | 52.7 |
| 3502 | | OD | | 0.00628 | 206 | 82.0 | 2690 | | | |
| 3503 | | OD | | 0.00900 | 209 | 35.6 | 827 | | | |
| 4001 | Male | OD | Subcutaneous | 0.05108 | 1022 | 362 | 7240 | 5430 | 2020 | 37.2 |
| 4002 | | OD | 15 mg/kg | 0.02330 | 466 | 163 | 3260 | | | |
| 4003 | | OD | | 0.05279 | 1056 | 290 | 5800 | | | |
| 4501 | Female | OD | | 0.06102 | 1220 | 1370 | 27400 | 18900 | 11300 | 59.8 |
| 4502 | | OD | | 0.04902 | 980 | 1160 | 23200 | | | |
| 4503 | | OD | | 0.02814 | 563 | 302 | 6040 | | | |
| 1004 | Male | OD | Subcutaneous | 0.09128 | 1091 | <LLOQ | <LLOQ | <LLOQ | ISD | ISD |
| 1005 | | OD | 0 mg/kg | 0.02906 | 581 | <LLOQ | <LLOQ | | | |
| 1504 | Female | OD | | 0.01454 | 291 | <LLOQ | <LLOQ | <LLOQ | ISD | ISD |
| 1505 | | OD | | 0.05920 | 1184 | <LLOQ | <LLOQ | | | |
| | | OD | | | | | | | | |
| 4004 | Male | | Subcutaneous | 0.02202 | 440 | 388 | 7750 | 4380 | ISD | ISD |
| 4005 | | OD | 15 mg/kg | 0.04424 | 885 | 50.5 | 1010 | | | |
| 4504 | Female | OD | | 0.00864 | 209 | 74.4 | 1800 | 4540 | ISD | ISD |
| 4505 | | OD | | 0.04202 | 840 | 364 | 7280 | | | |

LLOQ = 1.00 ng/mL, 20.0 ng/g
*Value confirmed with repeat analysis (1.58 ng/mL), original value reported.
ISD—Insufficient data for determination.

TABLE 9
Concentration of Compound of Formula IIa in Cynomolgus Monkey Sclera following SC Administration.

| | | | | | Total | | | Average | | |
|---|---|---|---|---|---|---|---|---|---|---|
| Animal | | | | Tissue | Volume | Concentration | Concentration | Concentration | Std. | |
| ID | Gender | Eye | Dose Route | Weight (g) | (μL) | (ng/mL) | (ng/g) | (ng/g) | Dev | % CV |
| 1001 | Male | OD | Subcutaneous | 0.15837 | 1583 | <LLOQ | <LLOQ | <LLOQ | ISD | ISD |
| 1002 | | OD | 0 mg/kg | 0.15404 | 1540 | <LLOQ | <LLOQ | | | |
| 1003 | | OD | | 0.20470 | 2047 | <LLOQ | <LLOQ | | | |
| 1501 | Female | OD | | 0.10905 | 1090 | <LLOQ | <LLOQ | <LLOQ | ISD | ISD |
| 1502 | | OD | | 0.13311 | 1331 | <LLOQ | <LLOQ | | | |
| 1503 | | OD | | 0.12368 | 1237 | <LLOQ | <LLOQ | | | |
| 2001 | Male | OD | Subcutaneous | 0.10724 | 1072 | 41.2 | 412 | 701 | 253 | 36.1 |
| 2002 | | OD | 2 mg/kg | 0.12749 | 1274 | 81.2 | 811 | | | |
| 2003 | | OD | | 0.12473 | 1248 | 88.0 | 880 | | | |
| 2501 | Female | OD | | 0.12880 | 1288 | 54.5 | 545 | 446 | 158 | 35.4 |
| 2502 | | OD | | 0.12382 | 1238 | 26.4 | 264 | | | |
| 2503 | | OD | | 0.11471 | 1147 | 52.8 | 528 | | | |
| 3001 | Male | OD | Subcutaneous | 0.15106 | 1511 | 105 | 1050 | 1820 | 676 | 37.1 |
| 3002 | | OD | 5 mg/kg | 0.17929 | 1793 | 212 | 2120 | | | |
| 3003 | | OD | | 0.13237 | 1323 | 230 | 2300 | | | |
| 3501 | Female | OD | | 0.14448 | 1444 | 73.5 | 735 | 837 | 668 | 79.8 |
| 3502 | | OD | | 0.13394 | 1339 | 22.6 | 226 | | | |
| 3503 | | OD | | 0.14243 | 1424 | 155 | 1550 | | | |
| 4001 | Male | OD | Subcutaneous | 0.13480 | 1348 | 200 | 2000 | 2260 | 227 | 10.0 |
| 4002 | | OD | 15 mg/kg | 0.13368 | 1337 | 236 | 2360 | | | |
| 4003 | | OD | | 0.11237 | 1123 | 242 | 2420 | | | |
| 4501 | Female | OD | | 0.17247 | 1724 | 1100 | 11000 | 7850 | 3620 | 46.1 |
| 4502 | | OD | | 0.09352 | 936 | 865 | 8660 | | | |
| 4503 | | OD | | 0.13316 | 1331 | 390 | 3900 | | | |
| 1004 | Male | OD | Subcutaneous | 0.14150 | 1416 | <LLOQ | <LLOQ | <LLOQ | ISD | ISD |
| 1005 | | OD | 0 mg/kg | 0.10316 | 1031 | <LLOQ | <LLOQ | | | |
| 1504 | Female | OD | | 0.11099 | 1110 | <LLOQ | <LLOQ | <LLOQ | ISD | ISD |
| 1505 | | OD | | 0.10879 | 1088 | <LLOQ | <LLOQ | | | |
| 4004 | Male | OD | Subcutaneous | 0.11519 | 1152 | 609 | 6090 | 3870 | ISD | ISD |
| 4005 | | OD | 15 mg/kg | 0.11358 | 1136 | 164 | 1640 | | | |
| 4504 | Female | OD | | 0.10497 | 1050 | 514 | 5140 | 4700 | ISD | ISD |
| 4505 | | OD | | 0.10101 | 1010 | 425 | 4250 | | | |

LLOQ = 1.00 ng/ml, 10.0 ng/g
ISD—Insufficient data for determination.

TABLE 10
Concentration of Compound of Formula IIa in Cynomolgus Monkey Cornea following SC Administration.

| | | | | | Total | | | | | |
|---|---|---|---|---|---|---|---|---|---|---|
| Animal | | | | Tissue | Volume | Concentration | Concentration | Average | Std. | |
| ID | Gender | Eye | Dose Route | Weight (g) | (μL) | (ng/mL) | (ng/g) | Conc. (ng/g) | Dev | % CV |
| 1001 | Male | OD | Subcutaneous | 0.03190 | 638 | <LLOQ | <LLOQ | <LLOQ | ISD | ISD |
| 1002 | | OD | 0 mg/kg | 0.04163 | 833 | <LLOQ | <LLOQ | | | |
| 1003 | | OD | | 0.04189 | 838 | <LLOQ | <LLOQ | | | |
| 1501 | Female | OD | | 0.03234 | 646 | <LLOQ | <LLOQ | <LLOQ | ISD | ISD |
| 1502 | | OD | | 0.04333 | 866 | <LLOQ | <LLOQ | | | |
| 1503 | | OD | | 0.03616 | 723 | <LLOQ | <LLOQ | | | |
| 2001 | Male | OD | Subcutaneous | 0.04171 | 834 | 2.47 | 49.4 | 39.3 | 13.5 | 34.4 |
| 2002 | | OD | 2 mg/kg | 0.04631 | 926 | 1.20 | 24.0 | | | |
| 2003 | | OD | | 0.03693 | 739 | 2.23 | 44.6 | | | |
| 2501 | Female | OD | | 0.03359 | 672 | 2.67 | 53.4 | 27.2 | 26.7 | 98.2 |
| 2502 | | OD | | 0.03786 | 757 | 1.41 | 28.2 | | | |
| 2503 | | OD | | 0.02741 | 548 | <LLOQ | <LLOQ | | | |
| 3001 | Male | OD | Subcutaneous | 0.04442 | 888 | 2.33 | 46.6 | 64.0 | 35.6 | 55.6 |
| 3002 | | OD | 5 mg/kg | 0.03917 | 783 | 2.02 | 40.4 | | | |
| 3003 | | OD | | 0.03535 | 707 | 5.24 | 105 | | | |
| 3501 | Female | OD | | 0.03692 | 738 | 2.08 | 41.6 | 56.1 | 25.6 | 45.6 |
| 3502 | | OD | | 0.03418 | 683 | 4.29 | 85.7 | | | |
| 3503 | | OD | | 0.02924 | 585 | 2.05 | 41.0 | | | |
| 4001 | Male | OD | Subcutaneous | 0.02906 | 581 | 6.37 | 127 | 195 | 83.2 | 42.7 |
| 4002 | | OD | 15 mg/kg | 0.02956 | 592 | 8.55 | 171 | | | |
| 4003 | | OD | | 0.03385 | 677 | 14.4 | 288 | | | |
| 4501 | Female | OD | | 0.05030 | 1006 | 169 | 3380 | 3110 | 2790 | 89.7 |
| 4502 | | OD | | 0.03667 | 734 | 288 | 5760 | | | |
| 4503 | | OD | | 0.03888 | 778 | 10.2 | 204 | | | |
| 1004 | Male | OD | Subcutaneous | 0.04224 | 845 | <LLOQ | <LLOQ | <LLOQ | ISD | ISD |
| 1005 | | OD | 0 mg/kg | 0.03978 | 796 | <LLOQ | <LLOQ | | | |
| 1504 | Female | OD | | 0.03373 | 675 | <LLOQ | <LLOQ | <LLOQ | ISD | ISD |
| 1505 | | OD | | 0.02377 | 476 | <LLOQ | <LLOQ | | | |
| | | OD | | | | | | | | |
| 4004 | Male | | Subcutaneous | 0.04040 | 808 | 1.73 | 34.6 | 34.8 | ISD | ISD |
| 4005 | | OD | 15 mg/kg | 0.03529 | 706 | 1.75 | 35.0 | | | |
| 4504 | Female | OD | | 0.02642 | 528 | 4.12 | 82.3 | 149 | ISD | ISD |
| 4505 | | OD | | 0.02139 | 427 | 10.8 | 216 | | | |

LLOQ = 1.00 ng/ml, 20.0 ng/g
ISD—Insufficient data for determination

TABLE 11
Concentration of Compound of Formula IIa in Cynomolgus Monkey Lens following SC Administration.

| | | | | | Total | | | Average | | |
|---|---|---|---|---|---|---|---|---|---|---|
| Animal | | | | Tissue | Volume | Concentration | Conc. | Concentration | Std. | |
| ID | Gender | Eye | Dose Route | Weight (g) | (μL) | (ng/mL) | (ng/g) | (ng/g) | Dev | %CV |
| 1001 | Male | OD | Subcutaneous | 0.09493 | 949 | <LLOQ | <LLOQ | <LLOQ | ISD | ISD |
| 1002 | | OD | 0 mg/kg | 0.08517 | 852 | <LLOQ | <LLOQ | | | |
| 1003 | | OD | | 0.09631 | 963 | <LLOQ | <LLOQ | | | |
| 1501 | Female | OD | | 0.09556 | 956 | <LLOQ | <LLOQ | <LLOQ | ISD | ISD |
| 1502 | | OD | | 0.09654 | 966 | <LLOQ | <LLOQ | | | |
| 1503 | | OD | | 0.08834 | 883 | <LLOQ | <LLOQ | | | |
| 2001 | Male | OD | Subcutaneous | 0.09484 | 949 | <LLOQ | <LLOQ | <LLOQ | ISD | ISD |
| 2002 | | OD | 2 mg/kg | 0.09088 | 909 | <LLOQ | <LLOQ | | | |
| 2003 | | OD | | 0.08764 | 877 | <LLOQ | <LLOQ | | | |
| 2501 | Female | OD | | 0.09939 | 994 | <LLOQ | <LLOQ | <LLOQ | ISD | ISD |
| 2502 | | OD | | 0.07119 | 712 | <LLOQ | <LLOQ | | | |
| 2503 | | OD | | 0.07500 | 750 | <LLOQ | <LLOQ | | | |
| 3001 | Male | OD | Subcutaneous | 0.09383 | 938 | <LLOQ | <LLOQ | <LLOQ | ISD | ISD |
| 3002 | | OD | 5 mg/kg | 0.09761 | 976 | <LLOQ | <LLOQ | | | |
| 3003 | | OD | | 0.10021 | 1002 | <LLOQ | <LLOQ | | | |
| 3501 | Female | OD | | 0.10242 | 1024 | <LLOQ | <LLOQ | <LLOQ | ISD | ISD |
| 3502 | | OD | | 0.09142 | 914 | <LLOQ | <LLOQ | | | |
| 3503 | | OD | | 0.09525 | 952 | <LLOQ | <LLOQ | | | |
| 4001 | Male | OD | Subcutaneous | 0.08820 | 882 | <LLOQ | <LLOQ | <LLOQ | ISD | ISD |
| 4002 | | OD | 15 mg/kg | 0.07775 | 778 | <LLOQ | <LLOQ | | | |
| 4003 | | OD | | 0.09096 | 910 | <LLOQ | <LLOQ | | | |
| 4501 | | OD | | 0.09261 | 926 | 2.12 | 21.2 | | | |
| 4502 | Female | OD | | 0.06685 | 669 | 1.10 | 11.0 | 10.7 | 10.6 | 99.1 |
| 4503 | | OD | | 0.09204 | 920 | <LLOQ | <LLOQ | | | |
| 1004 | Male | OD | Subcutaneous | 0.09181 | 918 | <LLOQ | <LLOQ | <LLOQ | ISD | ISD |
| 1005 | | OD | 0 mg/kg | 0.08856 | 886 | <LLOQ | <LLOQ | | | |
| 1504 | Female | OD | | 0.09176 | 918 | <LLOQ | <LLOQ | <LLOQ | ISD | ISD |
| 1505 | | OD | | 0.09182 | 918 | <LLOQ | <LLOQ | | | |
| 4004 | Male | OD | Subcutaneous | 0.10488 | 1049 | <LLOQ | <LLOQ | <LLOQ | ISD | ISD |
| 4005 | | OD | 15 mg/kg | 0.11726 | 1172 | <LLOQ | <LLOQ | | | |
| 4504 | Female | OD | | 0.07853 | 786 | <LLOQ | <LLOQ | <LLOQ | ISD | ISD |
| 4505 | | OD | | 0.07566 | 757 | <LLOQ | <LLOQ | | | |

LLOQ = 1.00 ng/ml, 10.0 ng/g
ISD—Insufficient data for determination

TABLE 12
Concentration of Compound of Formula IIa in Cynomolgus Monkey ICB following SC Administration.

| | | | | | Total | | | | | |
|---|---|---|---|---|---|---|---|---|---|---|
| Animal | | | | Tissue | Volume | Concentration | Concentration | Average | Std. | |
| ID | Gender | Eye | Dose Route | Weight (g) | (μL) | (ng/ml) | (ng/g) | Conc. (ng/g) | Dev | % CV |
| 1001 | Male | OD | Subcutaneous | 0.01944 | 291 | <LLOQ | <LLOQ | <LLOQ | ISD | ISD |
| 1002 | | OD | 0 mg/kg | 0.05012 | 752 | <LLOQ | <LLOQ | | | |
| 1003 | | OD | | 0.03473 | 521 | <LLOQ | <LLOQ | | | |
| 1501 | Female | OD | | 0.01891 | 284 | <LLOQ | <LLOQ | <LLOQ | ISD | ISD |
| 1502 | | OD | | 0.04499 | 675 | <LLOQ | <LLOQ | | | |
| 1503 | | OD | | 0.02953 | 443 | <LLOQ | <LLOQ | | | |
| 2001 | Male | OD | Subcutaneous | 0.01999 | 300 | 17.4 | 261 | 354 | 235 | 66.4 |
| 2002 | | OD | 2 mg/kg | 0.02079 | 312 | 12.0 | 180 | | | |
| 2003 | | OD | | 0.02774 | 416 | 41.5 | 622 | | | |
| 2501 | Female | OD | | 0.02366 | 355 | 44.0 | 660 | 375 | 252 | 67.2 |
| 2502 | | OD | | 0.03316 | 497 | 18.9 | 283 | | | |
| 2503 | | OD | | 0.02763 | 415 | 12.2 | 183 | | | |
| 3001 | Male | OD | Subcutaneous | 0.03359 | 504 | 63.1 | 947 | 1230 | 462 | 37.6 |
| 3002 | | OD | 5 mg/kg | 0.01911 | 287 | 64.7 | 972 | | | |
| 3003 | | OD | | 0.03852 | 578 | 117 | 1760 | | | |
| 3501 | | OD | | 0.02409 | 361 | 42.1 | 631 | | | |
| 3502 | Female | OD | | 0.02885 | 433 | 27.3 | 410 | 800 | 497 | 62.1 |
| 3503 | | OD | | 0.01935 | 290 | 90.6 | 1360 | | | |
| 4001 | Male | OD | Subcutaneous | 0.01940 | 291 | 119 | 1790 | 3910 | 2000 | 51.2 |
| 4002 | | OD | 15 mg/kg | 0.03403 | 510 | 385 | 5770 | | | |
| 4003 | | OD | | 0.01947 | 292 | 278 | 4170 | | | |
| 4501 | Female | OD | | 0.02435 | 365 | 600 | 8990 | 6870 | 2510 | 36.5 |
| 4502 | | OD | | 0.01804 | 271 | 273 | 4100 | | | |
| 4503 | | OD | | 0.03638 | 545 | 502 | 7520 | | | |
| 1004 | Male | OD | Subcutaneous | 0.03422 | 513 | <LLOQ | <LLOQ | <LLOQ | ISD | ISD |
| 1005 | | OD | 0 mg/kg | 0.03584 | 538 | <LLOQ | <LLOQ | | | |
| 1504 | Female | OD | | 0.04653 | 698 | <LLOQ | <LLOQ | <LLOQ | ISD | ISD |
| 1505 | | OD | | 0.02848 | 427 | <LLOQ | <LLOQ | | | |
| 4004 | Male | OD | Subcutaneous | 0.03600 | 540 | 99.1 | 1490 | 1410 | ISD | ISD |
| 4005 | | OD | 15 mg/kg | 0.03237 | 485 | 88.3 | 1320 | | | |
| 4504 | Female | OD | | 0.02727 | 409 | 333 | 4990 | 3160 | ISD | ISD |
| 4505 | | OD | | 0.02791 | 419 | 88.1 | 1320 | | | |

LLOQ = 1.00 ng/ml, 15.0 ng/g
ISD—Insufficient data for determination

TABLE 13
Concentration of Compound of Formula IIa in Cynomolgus Monkey Retina following SC Administration.

| | | | | | Total | | | | | |
|---|---|---|---|---|---|---|---|---|---|---|
| Animal | | | | Tissue | Volume | Concentration | Concentration | Average | Std. | |
| ID | Gender | Eye | Dose Route | Weight (g) | (μL) | (ng/mL) | (ng/g) | Conc. (ng/g) | Dev | % CV |
| 1001 | Male | OD | Subcutaneous | 0.07599 | 380 | <LLOQ | <LLOQ | <LLOQ | ISD | ISD |
| 1002 | | OD | 0 mg/kg | 0.09446 | 472 | <LLOQ | <LLOQ | | | |
| 1003 | | OD | | 0.11117 | 556 | <LLOQ | <LLOQ | | | |
| 1501 | Female | OD | | 0.05998 | 300 | <LLOQ | <LLOQ | <LLOQ | ISD | ISD |
| 1502 | | OD | | 0.05769 | 289 | <LLOQ | <LLOQ | | | |
| 1503 | | OD | | 0.04068 | 204 | <LLOQ | <LLOQ | | | |
| 2001 | Male | OD | Subcutaneous | 0.05432 | 271 | 896 | 4470 | 2770 | 1770 | 63.9 |
| 2002 | | OD | 2 mg/kg | 0.04763 | 239 | 188 | 943 | | | |
| 2003 | | OD | | 0.10395 | 520 | 580 | 2900 | | | |
| 2501 | Female | OD | | 0.07038 | 352 | 2010 | 10100 | 7390 | 3340 | 45.2 |
| 2502 | | OD | | 0.07006 | 350 | 733 | 3660 | | | |
| 2503 | | OD | | 0.05618 | 281 | 1680 | 8400 | | | |
| 3001 | Male | OD | Subcutaneous | 0.06378 | 319 | 758 | 3790 | 5760 | 1980 | 34.4 |
| 3002 | | OD | 5 mg/kg | 0.08489 | 425 | 1150 | 5760 | | | |
| 3003 | | OD | | 0.03324 | 166 | 1550 | 7740 | | | |
| 3501 | Female | OD | | 0.09487 | 474 | 2650 | 13200 | 20100 | 8500 | 42.3 |
| 3502 | | OD | | 0.05281 | 264 | 3500 | 17500 | | | |
| 3503 | | OD | | 0.05296 | 265 | 5910 | 29600 | | | |
| 4001 | Male | OD | Subcutaneous | 0.07686 | 384 | 4900 | 24500 | 26100 | 7780 | 29.8 |
| 4002 | | OD | 15 mg/kg | 0.05333 | 266 | 3870 | 19300 | | | |
| 4003 | | OD | | 0.06913 | 346 | 6910 | 34600 | | | |
| 4501 | Female | OD | | 0.06873 | 344 | 6970 | 34900 | 38900 | 20500 | 52.7 |
| 4502 | | OD | | 0.05969 | 299 | 12200 | 61100 | | | |
| 4503 | | OD | | 0.04306 | 215 | 4160 | 20800 | | | |
| 1004 | Male | OD | Subcutaneous | 0.06795 | 340 | <LLOQ | <LLOQ | <LLOQ | ISD | ISD |
| 1005 | | OD | 0 mg/kg | 0.07936 | 396 | <LLOQ | <LLOQ | | | |
| 1504 | Female | OD | | 0.04471 | 224 | <LLOQ | <LLOQ | <LLOQ | ISD | ISD |
| 1505 | | OD | | 0.05392 | 270 | <LLOQ | <LLOQ | | | |
| 4004 | Male | OD | Subcutaneous | 0.05407 | 270 | 8390 | 41900 | 30200 | ISD | ISD |
| 4005 | | OD | 15 mg/kg | 0.05926 | 296 | 3710 | 18500 | | | |
| 4504 | Female | OD | | 0.05076 | 254 | 11600 | 58000 | 67000 | ISD | ISD |
| 4505 | | OD | | 0.07635 | 381 | 15200 | 75900 | | | |

LLOQ = 1.00 ng/mL, 5.00 ng/g
ISD—Insufficient data for determination

TABLE 14
Concentration of Compound of Formula IIa in Cynomolgus Monkey Choroid following SC Administration.

| | | | | | Total | | | | | |
|---|---|---|---|---|---|---|---|---|---|---|
| Animal | | | | Tissue | Volume | Concentration | Concentration | Average | Std. | |
| ID | Gender | Eye | Dose Route | Weight (g) | (μL) | (ng/mL) | (ng/g) | Conc. (ng/g) | Dev | % CV |
| 1001 | Male | OD | Subcutaneous | 0.02511 | 377 | <LLOQ | <LLOQ | <LLOQ | ISD | ISD |
| 1002 | | OD | 0 mg/kg | 0.02507 | 376 | <LLOQ | <LLOQ | | | |
| 1003 | | OD | | 0.03129 | 469 | <LLOQ | <LLOQ | | | |
| 1501 | Female | OD | | 0.02538 | 380 | <LLOQ | <LLOQ | <LLOQ | ISD | ISD |
| 1502 | | OD | | 0.03532 | 529 | <LLOQ | <LLOQ | | | |
| 1503 | | OD | | 0.03159 | 474 | <LLOQ | <LLOQ | | | |
| 2001 | Male | OD | Subcutaneous | 0.01768 | 266 | 146 | 2200 | 1890 | 344 | 18.2 |
| 2002 | | OD | 2 mg/kg | 0.02540 | 381 | 101 | 1520 | | | |
| 2003 | | OD | | 0.02160 | 324 | 130 | 1950 | | | |
| 2501 | Female | OD | | 0.02538 | 380 | 380 | 5690 | 2500 | 2790 | 112 |
| 2502 | | OD | | 0.03876 | 582 | 35.9 | 539 | | | |
| 2503 | | OD | | 0.02732 | 409 | 84.0 | 1260 | | | |
| 3001 | Male | OD | Subcutaneous | 0.02404 | 361 | 139 | 2090 | 4060 | 2290 | 56.4 |
| 3002 | | OD | 5 mg/kg | 0.02852 | 428 | 234 | 3510 | | | |
| 3003 | | OD | | 0.02716 | 407 | 439 | 6580 | | | |
| 3501 | Female | OD | | 0.02509 | 376 | 411 | 6160 | 3210 | 2550 | 79.4 |
| 3502 | | OD | | 0.03345 | 501 | 108 | 1620 | | | |
| 3503 | | OD | | 0.03600 | 540 | 124 | 1860 | | | |
| 4001 | Male | OD | Subcutaneous | 0.03082 | 462 | 426 | 6390 | 8790 | 2140 | 24.3 |
| 4002 | | OD | 15 mg/kg | 0.02489 | 373 | 633 | 9490 | | | |
| 4003 | | OD | | 0.02370 | 356 | 700 | 10500 | | | |
| 4501 | Female | OD | | 0.03300 | 495 | 1080 | 16200 | 13000 | 3270 | 25.2 |
| 4502 | | OD | | 0.02288 | 343 | 881 | 13200 | | | |
| 4503 | | OD | | 0.02382 | 357 | 645 | 9670 | | | |
| 1004 | Male | OD | Subcutaneous | 0.02950 | 443 | <LLOQ | <LLOQ | <LLOQ | ISD | ISD |
| 1005 | | OD | 0 mg/kg | 0.02692 | 404 | <LLOQ | <LLOQ | | | |
| 1504 | Female | OD | | 0.02736 | 410 | <LLOQ | <LLOQ | <LLOQ | ISD | ISD |
| 1505 | | OD | | 0.01983 | 298 | <LLOQ | <LLOQ | | | |
| 4004 | Male | OD | Subcutaneous | 0.01977 | 297 | 488 | 7330 | 7880 | ISD | ISD |
| 4005 | | OD | 15 mg/kg | 0.02000 | 300 | 561 | 8420 | | | |
| 4504 | Female | OD | | 0.01896 | 284 | 2370 | 35500 | 27600 | ISD | ISD |
| 4505 | | OD | | 0.02223 | 333 | 1310 | 19600 | | | |

LLOQ = 1.00 ng/mL, 15.0 ng/g
ISD—Insufficient data for determination

TABLE 15
Concentration of Compound of Formula IIa in Cynomolgus Monkey Optic Nerve following SC Administration.

| | | | | | Total | | | | | |
|---|---|---|---|---|---|---|---|---|---|---|
| Animal | | | | Tissue | Volume | Concentration | Concentration | Average | Std. | |
| ID | Gender | Eye | Dose Route | Weight (g) | (μL) | (ng/ml) | (ng/g) | Conc. (ng/g) | Dev | % CV |
| 1001 | Male | OD | Subcutaneous | 0.12456 | 1246 | <LLOQ | <LLOQ | <LLOQ | ISD | ISD |
| 1002 | | OD | 0 mg/kg | 0.05551 | 556 | <LLOQ | <LLOQ | | | |
| 1003 | | OD | | 0.07305 | 730 | <LLOQ | <LLOQ | | | |
| 1501 | Female | OD | | 0.08786 | 879 | <LLOQ | <LLOQ | <LLOQ | ISD | ISD |
| 1502 | | OD | | 0.08249 | 824 | <LLOQ | <LLOQ | | | |
| 1503 | | OD | | 0.05285 | 529 | <LLOQ | <LLOQ | | | |
| 2001 | Male | OD | Subcutaneous | 0.07550 | 755 | 1740 | 17400 | 22000 | 11200 | 50.9 |
| 2002 | | OD | 2 mg/kg | 0.07007 | 701 | 1380 | 13800 | | | |
| 2003 | | OD | | 0.04615 | 461 | 3470 | 34700 | | | |
| 2501 | Female | OD | | 0.07818 | 782 | 1750 | 17500 | 17500 | 6550 | 37.4 |
| 2502 | | OD | | 0.02346 | 234 | 2410 | 24000 | | | |
| 2503 | | OD | | 0.00847 | 602 | 154 | 10900 | | | |
| 3001 | Male | OD | Subcutaneous | 0.08826 | 882 | 2460 | 24600 | 26300 | 3000 | 11.4 |
| 3002 | | OD | 5 mg/kg | 0.04265 | 427 | 2980 | 29800 | | | |
| 3003 | | OD | | 0.13915 | 1391 | 2460 | 24600 | | | |
| 3501 | Female | OD | | 0.06052 | 606 | 2950 | 29500 | 31800 | 2140 | 6.73 |
| 3502 | | OD | | 0.09825 | 982 | 3230 | 32300 | | | |
| 3503 | | OD | | 0.04694 | 469 | 3370 | 33700 | | | |
| 4001 | Male | OD | Subcutaneous | 0.05417 | 542 | 5480 | 54800 | 60000 | 4700 | 7.83 |
| 4002 | | OD | 15 mg/kg | 0.07051 | 706 | 6380 | 63900 | | | |
| 4003 | | OD | | 0.02809 | 281 | 6140 | 61400 | | | |
| 4501 | Female | OD | | 0.13749 | 1374 | 4800 | 48000 | 63100 | 13100 | 20.8 |
| 4502 | | OD | | 0.07085 | 709 | 7140 | 71500 | | | |
| 4503 | | OD | | 0.06603 | 660 | 6980 | 69800 | | | |
| 1004 | Male | OD | Subcutaneous | 0.04654 | 466 | <LLOQ | <LLOQ | <LLOQ | ISD | ISD |
| 1005 | | OD | 0 mg/kg | 0.02864 | 287 | <LLOQ | <LLOQ | | | |
| 1504 | Female | OD | | 0.07123 | 712 | <LLOQ | <LLOQ | <LLOQ | ISD | ISD |
| 1505 | | OD | | 0.05747 | 574 | <LLOQ | <LLOQ | | | |
| 4004 | Male | OD | Subcutaneous | 0.00789 | 602 | 1760 | 134000 | 91500 | ISD | ISD |
| 4005 | | OD | 15 mg/kg | 0.06702 | 670 | 4890 | 48900 | | | |
| 4504 | Female | OD | | 0.04604 | 460 | 4350 | 43500 | 40800 | ISD | ISD |
| 4505 | | OD | | 0.03756 | 376 | 3800 | 38000 | | | |

LLOQ = 5.00 ng/ml, 50.0 ng/g
ISD—Insufficient data for determination

### Summary

The data illustrates that compound of Formula IIa accumulates in a dose dependent manner in the ocular tissues of the cynomolgus monkey when administered subcutaneously. The concentrations attained (at 10s to 100s of ng/g, or more) would be expected to be sufficient to produce a therapeutic effect.

### Example 4—Uptake of Compound of Formula IIa in the Retina in a Non-Human Primate Model

This example demonstrates that compound of Formula IIa is taken up in concentrations expected to be sufficient to produce a therapeutic effect in the retina when administered by subcutaneous injection for 10 days to cynomolgus monkeys.

### Summary

The study design is summarized in Table 16.

TABLE 16
Experimental design.

| | | | | Dose | |
|---|---|---|---|---|---|
| | Test | Dose Level | Dose | Concentration | Animal^{b} |
| Group No. | Material | (mg/kg/day) | Volumeª | (mg/mL) | Number |
| 1 | Compound | 5 | 0.15 | 33.3 | 1001 |
| | of Formula | | | | 1002 |
| | IIa | | | | 1003 |

^{a}Based on the most recent body weight measurement.
^{b}Cynomolgus monkey; Animal supplier: Charles River Laboratories, 531 Boul des Prairies, Laval, QC H7V 1B7, Canada; Animal Origin: Asia; Number of Males: 3; Age at initiation of dosing: 18 to 60 months; Weight at initiation of dosing: 1.4. to 5.0 kg.

There was no mortality during the study. There were no differences in body weights noted throughout the course of this study that were considered to be related to the administration of compound of Formula IIa. Administration of compound of Formula IIa by subcutaneous injection was well-tolerated in cynomolgus monkeys at a level of 5 mg/kg/day.

### Methods

Animal screening and identification. Prior to transfer from the colony, all animals were subjected to a health assessment to ensure the animals were healthy and suitable for use in the study. Tattoo and/or subcutaneously implanted electronic identification chips were sued for animal identification.

Environmental acclimation. At least 5 days were allowed between animal transfer and surgical procedures. A period of at least 5 days was permitted between surgery and the start of dosing.

Husbandry. Each animal was housed in a separate stainless steel cage. Animals were separated during designated procedures/activities and were separated as required for monitoring and/or health purposes, as deemed appropriate by the study director or clinical veterinarian. The animal room environment was kept at a temperature ranging from 18° C. to 24° C., humidity of 30% to 70%, with 12 hours light and 12 hours dark (except during designated procedures). Animals were fed Envigo Teklad Certified Hi-Fiber Primate Diet #7195C twice daily, except during designated procedures. Animals were provided with freely available municipal tap water, treated by reverse osmosis and ultraviolet irradiation.

Veterinary care was available throughout the course of the study and animals were examined by veterinary staff as warranted by clinical signs or other changes.

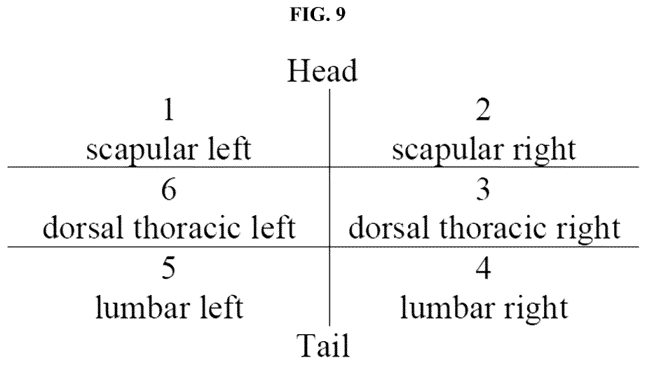

Administration of Compound of Formula IIa. The test agent (compound of formula IIa) was administered daily via subcutaneous injection into the scapular and mid-dorsal areas for 10 days. The first day of dosing was designated as Day 1. Dose formulations were allowed to warm up at ambient temperature for at least 30 minutes prior to dosing, as appropriate. The animals were temporarily restrained for dose administration and were not sedated. The volume for each dose was administered over 1 (preferred) or 2 (as necessary) separated injections within the designated area. The injection sites were rotated daily as shown in FIG. 9. If a designated injection site was not available for a given animal on any day, the next available test site in the rotation was used. The injection area was marked as frequently as required to allow appropriate visualization of administration sites. On the last injection occasion for each quadrant, the last site of injection was circled, and the circled site was collected for necropsy.

In-life procedures, observations, and measurements. Table 17 summarizes the general in-life assessments made of each animal.

TABLE 17
General in-life assessments-all animals.

| | Frequency | |
|---|---|---|
| Parameter | (minimum required) | Comments |
| Mortality | At least twice daily | Animals were observed |
| | (morning and afternoon) | within their cage unless |
| | beginning upon arrival | necessary for identification |
| | through termination. | or confirmation of findings. |
| Individual | Weekly; from at least | Fasted weight on the day of |
| Body Weights | Week −1 and then | necropsy. |
| | twice weekly thereafter | |

Method of euthanasia. The animals were sedated with intramuscular injection of a combination of ketamine hydrochloride and acepromazine, and then euthanized by intravenous overdose of sodium pentobarbital, followed by exsanguination.

Tissue collection, preservation, and analysis. Eye tissue (retina) was collected approximately 24 hours after the last dose on Day 10. The left side eye (retina) collected for biodistribution analysis was snap frozen in liquid nitrogen, placed in dry ice, and stored at −70° C.

Tissue samples were prepared and analyzed by methods similar to those described above in Example 3. In brief, concentrations of test article (compound of Formula IIa) in the retina (ng/g tissue) was determined by LC-MS/MS using Formula IIb as a standard.

### Results

The results are shown in Table 18.

TABLE 18
Concentration of Compound of
Formula IIa in Retina at Day 11.

| Subject | Concentration (ng/g) |
|---|---|
| 1001 | 382 |
| 1002 | 336 |
| 1003 | 126 |

### Summary

The data further illustrates that compound of Formula IIa accumulates in the retina of the cynomolgus monkey when administered subcutaneously. The concentrations attained (at 100s of ng/g) would be expected to be sufficient to produce a therapeutic effect.

### EQUIVALENTS

The present technology is not to be limited in terms of the particular embodiments described in this application, which are intended as single illustrations of individual aspects of the present technology. Many modifications and variations of this technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that the present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. An ophthalmic topical composition for administration to one or more eyes of a mammalian subject in need thereof, said composition comprising a peptidomimetic of Formula II or a pharmaceutically acceptable salt, stereoisomer, tautomer, hydrate, and/or solvate thereof: 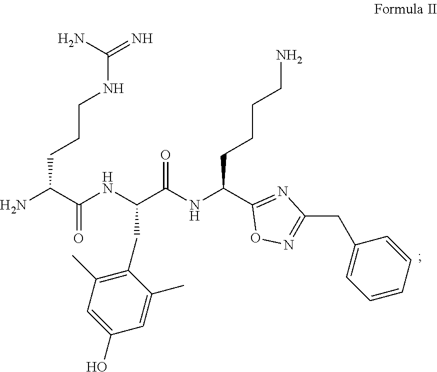 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
wherein one or more of the hydrogen atoms of the peptidomimetic is/are optionally substituted with a deuterium or fluorine atom, and
(a) the peptidomimetic is present in the topical ophthalmic composition at a concentration of between 1 and 2% (wt./vol.), inclusive;
(b) the peptidomimetic is present in the topical ophthalmic composition at a concentration of between 2 and 3% (wt./vol.), inclusive; or
(c) the peptidomimetic is present in the topical ophthalmic composition at a concentration of between 3 and 5% (wt./vol.), inclusive.

2. The ophthalmic topical composition of claim 1, wherein the composition is formulated for administration as an eye drop to the subject.

3. The ophthalmic topical composition of claim 2, wherein the subject is human.

4. The ophthalmic topical composition of claim 1, wherein the topical ophthalmic composition is formulated for treating, inhibiting, ameliorating or delaying the onset of:
(i) an ophthalmic disease, disorder or condition of the subject; or
(ii) deterioration of ellipsoid zone integrity in one or more eyes of the subject.

5. The ophthalmic topical composition of claim 4, wherein the ophthalmic disease, disorder or condition is macular degeneration, dry age-related macular degeneration, wet age-related macular degeneration, geographic atrophy, diabetic retinopathy, diabetic macular edema, cataracts, autosomal dominant optic atrophy (DOA), Leber hereditary optic neuropathy (LHON), pigmentary retinopathy, retinitis pigmentosa, glaucoma, ocular hypertension, uveitis, chronic progressive external ophthalmoplegia (e.g., Kearns-Sayre syndrome), or Leber congenital amaurosis (LCA).

6. The ophthalmic topical composition of claim 5 wherein the subject is human.

7. The ophthalmic topical composition of claim 4, wherein the ophthalmic disease, disorder or condition is macular degeneration, dry age-related macular degeneration, wet age-related macular degeneration, geographic atrophy, diabetic retinopathy, diabetic macular edema, pigmentary retinopathy, retinitis pigmentosa, or glaucoma.

8. The ophthalmic topical composition of claim 4, wherein the ophthalmic disease, disorder or condition is dry age-related macular degeneration, geographic atrophy, diabetic retinopathy, retinitis pigmentosa, or glaucoma.

9. The ophthalmic topical composition of claim 8, wherein the subject is human.

10. The ophthalmic topical composition of claim 4, wherein the ophthalmic disease, disorder or condition is dry age-related macular degeneration.

11. The ophthalmic topical composition of claim 10, wherein the subject is human.

12. The ophthalmic topical composition of claim 4, wherein the topically ophthalmic composition further comprises a therapeutic agent selected from the group consisting of: an antioxidant, a metal complexer, an anti-inflammatory drug, an antibiotic, and an antihistamine.

13. The ophthalmic topical composition of claim 12, wherein the therapeutic agent is selected from the group consisting of: α-lipoic acid, aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, chrysoeriol, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, metformin, methazolamide, metipranolol, moxifloxacin, naphazoline, natamycin, necrostatins, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, PU-61, ranibizumab, resveratrol, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, ZLN005, pharmaceutically acceptable salts thereof, and combinations of two or more of the forgoing.

14. The ophthalmic topical composition of claim 12, wherein the therapeutic agent is selected from the group consisting of: carbachiol (Carbastat® or Carboptic®), Polocarpine (Salagen®), timolol (Timoptic®), betaxolol (Betoptic® or Keflone®), Carteolol (Cartrol® or Ocupress®), Levobunolol (Liquifilm®), brimonidine (Lumify® or Mirvaso®), apraclonidine (Iopidine®), latanoprost (Xalantan®), travoprost (Travatan®), bimatoprost (Lumigan®), talfluprost (Taflotan®), unoprostone isopropyl (Rescula®), dorzolamide (Trusopt®), brinzolamide (Azopt®), acetazolamide (Diamox®), methazolamide (Neptazane®), brimonidine tartrate/timolol maleate (Combigan®), timolo-dorzolamide (Cosopt®), travoprost-timolol (DuoTrav®), latanoprost and timolol maleate (Xalacom®).

15. An ophthalmic topical composition for administration to one or more eyes of a mammalian subject in need thereof, said composition comprising a peptidomimetic of Formula II or a pharmaceutically acceptable salt thereof: 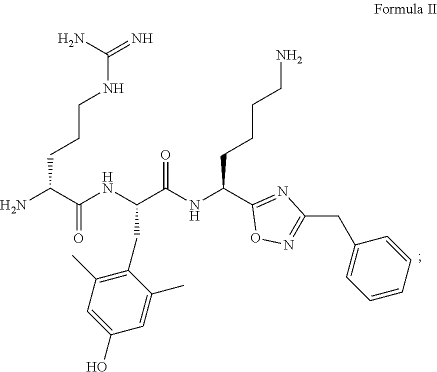 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
wherein one or more of the hydrogen atoms of the peptidomimetic is/are optionally substituted with a deuterium or fluorine atom, and the peptidomimetic is present in the topical ophthalmic composition at a concentration of between 1 and 2% (wt./vol.), inclusive.

16. The ophthalmic topical composition of claim 15, wherein the composition is formulated for administration as an eye drop to the subject.

17. The ophthalmic topical composition of claim 16, wherein the subject is human.

18. An ophthalmic topical composition for administration to one or more eyes of a mammalian subject in need thereof, said composition comprising a peptidomimetic of Formula II or a pharmaceutically acceptable salt thereof: 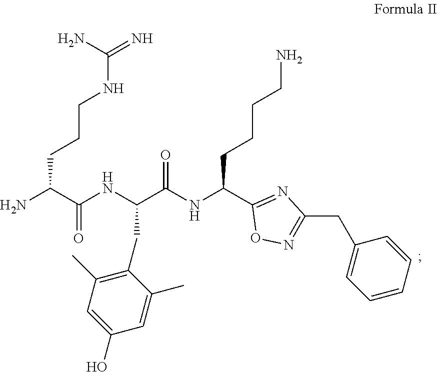 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
wherein one or more of the hydrogen atoms of the peptidomimetic is/are optionally substituted with a deuterium or fluorine atom, and the peptidomimetic is present in the topical ophthalmic composition at a concentration of between 2 and 3% (wt./vol.).

19. The ophthalmic topical composition of claim 18, wherein the composition is formulated for administration as an eye drop to the subject.

20. The ophthalmic topical composition of claim 19, wherein the subject is human.

21. An ophthalmic topical composition for administration to one or more eyes of a mammalian subject in need thereof, said composition comprising a peptidomimetic of Formula II or a pharmaceutically acceptable salt thereof: 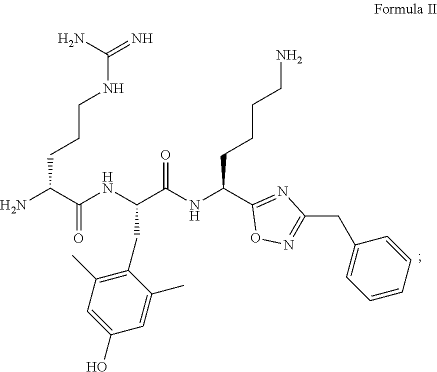 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
wherein one or more of the hydrogen atoms of the peptidomimetic is/are optionally substituted with a deuterium or fluorine atom, and the peptidomimetic is present in the topical ophthalmic composition at a concentration of between 3 and 5% (wt./vol.), inclusive.

22. The ophthalmic topical composition of claim 21, wherein the composition is formulated for administration as an eye drop to the subject.

23. The ophthalmic topical composition of claim 22, wherein the subject is human.
